(19) United States
(12) Patent Application Publication
(10) Pub. No.: US 2025/0152744 A1
(43) Pub. Date: May 15, 2025

(54) METHODS OF TREATING NEURODEGENERATIVE DISORDERS

(71) Applicant: Genzyme Corporation, Cambridge, MA (US)

(72) Inventor: Shyam RAMACHANDRAN, Winchester, MA (US)

(21) Appl. No.: 18/949,540

(22) Filed: Nov. 15, 2024

Related U.S. Application Data
(60) Provisional application No. 63/599,469, filed on Nov. 15, 2023.

(51) Int. Cl. A61K 48/00; A61K 38/47; A61P 25/28; C12N 9/24; C12N 15/86

(52) U.S. Cl. CPC A61K 48/0058; A61K 38/47; A61K 48/0075; A61P 25/28; C12N 9/2402; C12N 15/86; C12Y 302/01045; C12N 2750/14122; C12N 2750/14143; C12N 2750/14171; C12N 2830/48

(57) ABSTRACT

Provided herein are expression cassettes for expressing a transgene in a cell, wherein the transgene encodes a disorder-related polypeptide. Also provided are methods to treat various neurodegenerative disorders. Further provided herein are vectors (e.g., rAAV vectors), viral particles, pharmaceutical compositions and kits for expressing a disorder-related polypeptide in an individual in need thereof.

### CROSS-REFERENCE TO RELATED APPLICATIONS

This application claims the priority benefit of U.S. Provisional Application No. 63/599,469, filed Nov. 15, 2023, which is incorporated by reference in its entirety.

### REFERENCE TO AN ELECTRONIC SEQUENCE LISTING

The content of the electronic sequence listing (159792018600seqlist.xml; Size: 46,176 bytes; and Date of Creation: Nov. 13, 2024) is herein incorporated by reference in its entirety.

### FIELD OF THE INVENTION

The present disclosure relates to methods for treating neurodegenerative disorders in a patient in need thereof, the methods comprising administering to the cerebrospinal fluid (CSF) of a patient a recombinant adeno-associated virus (rAAV) viral particle.

### BACKGROUND

Millions of people are affected by neurodegenerative disorders (e.g., neurogenerative disease) across the world. Neurodegenerative disorders are characterized by loss of function of nerve cells in the brain or peripheral nervous system.

Currently, adeno-associated virus (AAV) vectors are used for gene delivery and treatment of human diseases. Applications include AAV gene therapy for central nervous system (CNS) disorders. AAV vectors do have drawbacks, however, including toxicity and biodistribution limitations, resulting in safety and dosing issues. Providing therapeutics to counter these drawbacks would be a highly valuable property for CNS gene therapy applications.

### BRIEF SUMMARY OF THE INVENTION

Described herein are various methods and compositions based in part on the development of rAAV viral particles comprising (a) an rAAV vector comprising an expression cassette that encodes a disorder-related polypeptide and/or an RNAi molecule, and (b) a modified AAV9 capsid capable of transducing the cells of the central nervous system (CNS). The term disorder-related polypeptide, as used herein refers to a polypeptide (e.g., an enzyme) implicated in a particular neurodegenerative disorder as disclosed herein. For instance, the rAAV particles of the disclosure can express an enzyme in the brain or central nervous system (CNS) that the patient lacks or is deficient in. Alternatively, an RNAi molecule may prevent or reduce the expression of a particular enzyme that is implicated in the neurodegenerative pathology. In some embodiments, the method may comprise administering viral particles into the CSF to provide high levels of expression of the target (e.g., disease-related polypeptide or RNAi molecule) in the CNS with low vector genome (VG) levels, while simultaneously providing low levels of expression in off-target peripheral nervous system (PNS) tissues. In some embodiments, the method may comprise administering viral particles into the CSF to provide high levels biodistribution across target tissue region.

Accordingly, the disclosure provides methods for treating or improving symptoms associated with a neurodegenerative disorder (e.g., central nervous system (CNS) disorder) in patients in need thereof, said methods comprising administering to the patient a recombinant adeno-associated virus (rAAV) viral particle comprising (a) an expression cassette comprising a vector encoding a disorder-related polypeptide (e.g., disorder-related protein) and/or an RNAi molecule capable of inducing interference in a cell; and (b) a modified AAV9 capsid protein as set forth herein.

In some embodiments, the neurodegenerative disorder is Parkinson's Disease (PD), Gaucher Disease (GD) Type 2 or Type 3, Alzheimer's Disease, Metachromatic leukodystrophy, Adrenoleukodystrophy, Amyotrophic lateral sclerosis and frontotemporal dementia, neuropathic pain, Charcot-Marie-Tooth Diseases, Fabry Disease, Angelman Syndrome, Fragile X Syndrome, Rett Syndrome, Tuberous Sclerosis Complex, Neurofibromatosis type II, Pompe, Globoid cell leukodystrophy, Multiple Sclerosis, or GBA-Parkinson's Disease (GBA-PD)

In some embodiments, a disorder-related gene (e.g., transgene) expresses the disorder-related polypeptide (e.g., disorder-related polypeptide of interest). In some embodiments, the disorder-related gene (e.g., transgene) expresses an RNAi molecule. In some embodiments, the RNAi molecule is an artificial microRNA (artificial miRNA or amiRNA). In some embodiments, the RNAi molecule is a small interfering RNA (siRNA). In some embodiments, the RNAi molecule is s small hairpin RNA (shRNA).

The modified AAV9 capsid proteins of the AAV viral particles comprise targeting peptides inserted into the AAV9 capsid that alter the transduction and/or endosomal release of the viral particle following administration to the patient. The rAAV particles comprising modified AAV9 capsid proteins, as disclosed herein, comprise three structural capsid proteins, VP1, VP2 and VP3. The three capsid proteins are alternative splice variants. In some embodiments, the targeting peptide is inserted into the VP1, VP2 and VP3 capsid proteins within the rAAV particle.

In particular embodiments, the targeting peptide of the modified AAV9 capsids are inserted after residue 588 of the AAV9 structural protein (numbering based on VP1 numbering of AAV9). In some embodiments, the targeting peptide has SEQ ID NO: 10. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 11. In some embodiments, the full modified AAV9 capsid structural protein has SEQ ID NO: 12. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 12, wherein the modified AAV9 structural capsid comprises the targeting peptide of SEQ ID NO: 10. The capsid having SEQ ID NO: 12 will also be referred to herein as SAN006 or AAV.SAN006

In some aspects, the rAAV viral particle is used to treat the neurodegenerative disorder (e.g., CNS disorder). In some embodiments, the rAAV viral particle can be administered to the cerebrospinal fluid (CSF) of the CNS disorder patient. In some embodiments, the viral particle is administered directly by intra-CSF administration of the patient with the CNS disorder. In some embodiments of the above aspects, the rAAV is administered via direct injection into the spinal cord, via intrathecal injection, or via intracisternal injection of the patient with the neurodegenerative disorder. In some embodiments, the rAAV is administered to more than one location of the spinal cord or cisterna magna of the patient with the neurodegenerative disorder. In some embodiments, the rAAV is administered to more than one location of the spinal cord of the patient with the neurodegenerative disorder. In some embodiments, the rAAV is administered to one or more of a lumbar subarachnoid space, thoracic subarachnoid space and a cervical subarachnoid space of the spinal cord of the patient with the neurodegenerative disorder. In some embodiments, the rAAV is administered to the cisterna magna of the patient with the neurodegenerative disorder. In some embodiments, the method may comprise treating the neurodegenerative disorder in a patient in need thereof. In particular embodiments, the expression cassette of the viral particle is able to drive transgene expression in the central nervous systems to treat the neurodegenerative disorder. In some embodiments, administration of the rAAV particles ameliorates symptoms associated with the neurodegenerative disorder. For instance, administration of the viral particle may reduce or impede the progression of brainstem and cortical dysfunction, seizures and cognitive defects.

In some embodiments, administration of the viral particle reduces or clears accumulated toxic lipid substrates in the brain of the neurodegenerative disorder patient. In some embodiments, administration of the viral particle reduces α-synuclein in the brain of the patient.

In some embodiments, transgene expression is further enhanced by the addition of the woodchuck hepatitis virus post-transcriptional regulatory element (WPRE). In some embodiments, the vector comprises a Chicken β-actin (CBA) promoter. In some embodiments, the vector comprises a WPRE element and a CBA promoter.

In some embodiments of the above aspects, the rAAV particle comprises a vector comprising an expression cassette flanked by one or more AAV inverted terminal repeat (ITR) sequences. In some embodiments, the expression cassette is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector is a self-complimenting vector. In some embodiments, the vector comprises first nucleic acid sequence encoding the disorder-related polypeptide and a second nucleic acid sequence encoding a complement of the disorder-related polypeptide, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence.

In some aspects, the disclosure provides a composition comprising any of the rAAV particles described herein. In some embodiments, the composition further comprises a pharmaceutically acceptable carrier.

In some aspects, the disclosure provides a cell comprising any of the rAAV particles described herein. In some aspects, the disclosure provides a method of producing a disorder-related polypeptide, the method comprising culturing a cell as described herein under conditions to produce the disorder-related polypeptide. In some embodiments, the methods further comprise the step of purifying the disorder-related polypeptide.

In some aspects, the disclosure provides methods for treating the neurodegenerative disorder in an individual in need thereof, comprising administering to the individual a rAAV particle as described herein. In some aspects, the disclosure provides methods for treating the neurodegenerative disorder in an individual in need thereof, comprising administering to the individual a composition as described herein. In some embodiments, the disclosure provides methods for treating the neurodegenerative disorder in an individual in need thereof, comprising administering to the individual the cell as described herein. In some embodiments, the individual lacks activity of a disorder-related gene.

In some embodiments of the above aspects, the rAAV particle is administered only one time to a patient in need thereof. In some embodiments, the rAAV particle is administered multiple times to a patient in need thereof (e.g., over one or more months or years). In other embodiments, the rAAV particle is administered once every year to a patient in need thereof. In other embodiments, the rAAV particle is administered twice yearly to a patient in need thereof.

In some embodiments, the disclosure provides kits comprising any of the rAAV particles, the compositions, or the cell as described herein. In some embodiments, the kit further comprises instructions for use; buffers and/or pharmaceutically acceptable excipients; and/or bottles, vials and/or syringes.

### BRIEF DESCRIPTION OF THE DRAWINGS

FIG. 1A shows a comparison between AAV-rh10, AAV.SAN006 and AAV-Myo in terms of biodistribution in the brain of non-human primates (NHP). FIG. 1B shows a plot of the percent increase in transgene expression in the brain of NHPs.

FIG. 2 shows a heatmap of the percent increase of gene expression by AAV.SAN006 over AAV.rh10 across an NHP brain.

FIG. 3 shows a broad and higher level of GFP expression of AAV.SAN006 versus AAVrh10 in an NHP brain.

FIG. 4A shows higher GFP expression in spinal cord, for AAV.SAN006 compared to AAV-Rh10. FIG. 4B shows similar expression levels observed in the dorsal root ganglia (DRG).

FIG. 5 shows GFP expression in NHP spinal cord and DRGs with AAV.SAN006-GFP.

FIG. 6A shows lower GFP expression as measured by ELISA in the heart for AAV.

SAN006 as compared to AAV-Rh10. FIG. 6B shows similar results in the liver.

FIG. 7A shows lower vector genome levels for AAV.SAN006 as compared to AARrh10 in the brain, spinal cord and DRG of NHPs. FIG. 7B shows vector genome levels in peripheral organs.

FIG. 8A shows GFP expression correlated with vector genome levels in an NHP brain.

FIG. 8B shows vector genome exposure in the brain regions. FIG. 8C shows expression levels in the brain.

FIG. 9A shows widespread vector biodistribution for AAV.SAN006 across 20 brain regions using three different intra-CSF routes of administration (ROA). FIG. 9B shows similar results for the transgene activity of AAV.SAN006 across the same 20 brain regions using the three different intra-CSF ROAs.

FIGS. 10A-10B show widespread vector biodistribution for AAV.SAN006 in the spinal cord and DRG, respectively, using three different intra-CSF ROAs. FIGS. 10C-10D show nanoluciferase transgene activity in the spinal cord and DRG, respectively, using three different intra-CSF ROAs.

FIG. 11 shows widespread vector biodistribution for AAV.SAN006 in peripheral tissues using three different intra-CSF ROAs.

FIG. 12 shows the impact of AAV.SAN006 on CSF NfL using three different intra-CSF ROAs.

FIGS. 13A-13F show histopathological findings in brain, spinal cord, and DRG following intra-CSF administration of AAV.SAN006.

FIG. 14 shows biodistribution for AAV.SAN006 in NHP peripheral tissues at 3 weeks post intravenous dosing.

FIGS. 15A-15B show GFP mRNA transcript levels and corresponding GFP ELISA, respectively, in peripheral tissues of NHPs injected with AAV.SAN006 GFP via intravenous dosing at 3 weeks post dosing.

FIGS. 16A-16C show efficient substrate clearance by SS3-GBA1 variant in multiple brain regions distal to site of injection. FIG. 16A depicts WPRE mRNA in situ hybridization showing AAV distribution around the ventricles. FIG. 16B depicts 4 month old WT mice injected with 1e11 VG each of the WT or SS3-GBA1 AAV packaged in SAN006. The viruses were allowed to express for 4 weeks and the mice were IP injected with 100 mg/kg CBE (conduritol β-epoxide) 24 hours prior to necropsy. Lyso-GL1 (FIG. 16B) and total GL-1 (FIG. 16C) in cerebellum, hindbrain and midbrain of the injected mice. No CBE group is naïve WT mice with neither virus nor CBE injected. N=8 mice per group. *** p<0.001, ** p<0.01, *p<0.05; One-way ANOVA with Tukey's multiple comparison test.

FIGS. 17A-17E depict effective reduction of GL1 lipids in SS3-GBA1 treated NHPs. FIG. 17A depicts Lyso-G11 in plasma spiked across all NHPs post CBE dosing. FIG. 17B depicts vector genomes and FIG. 17C huGBA1 transgene expression in vehicle treated, WT-GBA1 and SS3-GBA1 treated NHPs. 3 NHPs in vehicle and WT-GBA1 groups and 4 NHPs in SS3-GBA1 group. NHPs were dosed with 1.25e13 VGs via iCM dosing, 6 weeks in-life and injected with 30 mg/kg CBE (IV dosing) 48 hours before necropsy. Median with inter-quartile range, each data point is average of all NHPs in the group across 64 brain punches, representing 17 gray mater regions and 7 white mater regions. FIG. 17D depicts Lyso-GL1 and FIG. 17E depicts C18 GL1 across 47 grey mater brain punches. Each data point is average of all NHPs in the group for that punch. *** p<0.001; Two-way ANOVA with Tukey's multiple comparison test.

FIGS. 18A-18B depict effective lipid clearance in mice injected with SS3-GBA1 intravenously. FIG. 18A depicts 3 month old WT mice were injected with 4e13 VG/kg of vehicle or SS3-GBA1 intravenously. AAVs were expressed for 4 weeks followed by 100 mg/kg CBE (IP injection) 24 hours prior to necropsy. Lyso-GL1 across major peripheral tissues such as liver, spleen, kidney and lung. FIG. 18B depicts Lyso-GL1 across multiple muscles surveyed such as heart, diaphragm, quadriceps and gastrocnemius. No CBE group is naïve WT mice with neither virus nor CBE injected. N=8 mice per group. *** p<0.001; One-way ANOVA with Tukey's multiple comparison test.

FIGS. 19A-19B depict a strategy to generate engineered GBA1 variants and in vitro validation of constructs. FIG. 19A depicts a strategy to generate GBA1 variants with enhanced secretion. Endogenous signal sequence of human GBA1 was swapped with signal sequences from highly secreted proteins. Top 4 signal sequences (SS) were narrowed down using in-silico tools that predicted robust secretion as well as high (>98%) probably of cleavage at the end of signal sequence. FIG. 19B depicts cells transfected GBA containing the indicated SS variants were lysed and GCase enzyme activity was determined in cell lysates. Mean+SEM, each color represents data point from a single experiment. Untransfected versus all GBA1 constructs as well as GFP transfected versus all GBA1 constructs: *** p<0.001; One way-ANOVA with Tukey's multiple comparison test.

FIGS. 20A-20B depict robust secretion and parenchymal diffusion of GBA1 protein from AAV.SAN006 GBA1 viruses in vivo. FIG. 20A depicts vector biodistribution with in situ hybridization to WPRE (top panels) and huGBA1 immunohistochemistry (bottom panels) in sagittal sections of WT mice injected with AAV.SAN006-GBA1 variants (Bilateral ICV, 1e11 VG/mouse, 5 μl per hemisphere) and analyzed after 4 weeks of expression. FIG. 20B depicts higher magnification images of WPRE mRNA and GBA1 protein in SS3-GBA1 injected mice. Image corresponds to the purple box in FIG. 20A. Representative cells positive for both WPRE mRNA and GBA1 protein are shown in red arrows (AAV-transduced cells) while mRNA negative and GBA1 protein positive cells are shown in green (cross-corrected cells).

FIGS. 21A-21C show efficient substrate clearance by SS3-GBA1 variant in multiple brain regions distal to site of AAV injection. FIG. 21A depicts WPRE mRNA in situ hybridization showing AAV distribution around the ventricles. FIGS. 21B-21C depict 4 month old WT mice injected with 1e11 VG each of the AAV.SAN006-GBA1 variants were IP injected with 100 mg/kg CBE (conduritol β-epoxide) 24 hours prior to necropsy. Lyso-GL1 (FIG. 21B) and total GL-1 (FIG. 21A) in cerebellum, hindbrain and midbrain of the injected mice. “No CBE” group in the graphs shown is control mice that did not receive any virus or CBE injection. N=8 mice per group. *** p<0.001, ** p<0.01, *p<0.05; One-way ANOVA with Tukey's multiple comparison test with all groups compared to CBE-treated vehicle injected group.

FIGS. 22A-22E depict development of CBE-induced lipid flux model in NHPs. FIG. 22A depicts an illustration of study design of CBE administration in NHPs. FIG. 22B depicts Lyso-GL1 in plasma samples prior to CBE dosing and 24 hours post CBE (left) and dose-dependent increase in Lyso-GL1 in liver tissue homogenates (right). FIG. 22C shows that a dose-dependent increase in Lyso-GL1 was observed in liver from these NHPs FIGS. 22D-22E depict dose-dependent increase in Lyso-GL1 (FIG. 22D) and concomitant decrease in GCase enzyme activity (FIG. 22E) in 47 grey mater punches of NHP brains, representing 20 grey mater regions. N=1 NHP per dose. Median with inter-quartile range across 47 punches. *** p<0.001; Two-way ANOVA with Tukey's multiple comparison test with all groups compared to 0 mg/kg CBE group or no CBE group.

FIGS. 23A-23E depict effective reduction of GL1 lipids in SS3-GBA1 treated NHPs. FIG. 23A depicts Lyso-GL1 in plasma spiked across all NHPs post CBE dosing. FIGS. 23B-23C depict vector genomes and huGBA1 transgene expression, respectively, in vehicle treated, WT-GBA1 and SS3-GBA1 treated NHPs. 3 NHPs in vehicle and WT-GBA1 groups and 4 NHPs in SS3-GBA1 group. NHPs were dosed with 1.25e13 VGs via iCM dosing, 6 weeks in-life and injected with 30 mg/kg CBE (IV dosing) 48 hours before necropsy. Median with inter-quartile range, each data point is average of all NHPs in the group across 64 brain punches, representing 17 gray mater regions and 7 white mater regions. FIGS. 23D-23E depict Lyso-GL1 and C18 GL1, respectively, across 47 grey mater brain punches. Each data point is average of all NHPs in the group for that punch. *** p<0.001; Two-way ANOVA with Tukey's multiple comparison test. Purple line indicates lipid levels under physiological conditions.

FIGS. 24A-24B depict robust secretion and diffusion of SS3-GBA1 in brain sections of SS3-GBA1 iCM dosed NHPs. FIG. 24A depicts low magnification images of WPRE mRNA in situ hybridization (top panel) and huGBA1 immunohistochemistry (bottom panel) in representative NHP brain sections. FIG. 24B depicts higher magnification of insets from FIG. 24A of mRNA (left) and GBA1 protein (right). Cells positive for both WPRE mRNA and GBA1 protein are shown in red arrows (AAV-transduced cells) while mRNA negative and GBA1 protein positive cells are shown in green (cross-corrected cells).

### DETAILED DESCRIPTION

### Introduction

The disclosure provides pharmaceutical compositions comprising rAAV viral particles encapsulating an rAAV vector comprising a transgene capable of encoding a disorder-related polypeptide and/or an RNAi molecule capable of reducing or eliminating the expression of a target polypeptide (e.g., enzyme) implicated in a neurodegenerative disease. In some aspects, the disclosure provides expression cassettes, recombinant adeno-associated virus (rAAV) vectors, and viral particles and pharmaceutical compositions comprising a transgene encoding a disorder-related polypeptide and/or an RNAi molecule. The rAAV particles of the disclosure can be administered to patients suffering from one or more neurodegenerative disorders. Particular neurodegenerative disorders include, but are not limited to, Parkinson's Disease (PD), Gaucher Disease (GD) Type 2 or Type 3, Alzheimer's Disease, Metachromatic leukodystrophy, Adrenoleukodystrophy, Amyotrophic lateral sclerosis and frontotemporal dementia, neuropathic pain, Charcot-Marie-Tooth Diseases, Fabry Disease, Angelman Syndrome, Fragile X Syndrome, Rett Syndrome, Tuberous Sclerosis Complex, Neurofibromatosis type II, Pompe, Globoid cell leukodystrophy, Multiple Sclerosis, and GBA-Parkinson's Disease (GBA-PD)

### Definitions

A “vector,” as used herein, refers to a recombinant plasmid or virus that comprises a nucleic acid to be delivered into a host cell, either in vitro or in vivo.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one and in some embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, and in some embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

“Chicken β-actin (CBA) promoter” refers to a polynucleotide sequence derived from a chicken β-actin gene (e.g., Gallus beta actin, represented by GenBank Entrez Gene ID 396526). As used herein, “chicken β-actin promoter” may refer to a promoter containing a cytomegalovirus (CMV) early enhancer element, the promoter and first exon and intron of the chicken β-actin gene, and the splice acceptor of the rabbit beta-globin gene, such as the sequences described in Miyazaki, J. et al. (1989) Gene 79 (2): 269-77. As used herein, the term “CAG promoter” may be used interchangeably. As used herein, the term “CMV early enhancer/chicken beta actin (CAG) promoter” may be used interchangeably.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (SAN006) Hum. Gene Ther., 10:1031-1039; Veldwijk et al. (2002) Mol. Ther., 6:272-278.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in Mclaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include ElA functions, ElB functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 102:1; at least about 104:1, at least about 106:1; or at least about 108:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this disclosure, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the disclosure described herein include “comprising,” “consisting,” and/or “consisting essentially of” aspects and embodiments.

### Expression Cassettes

In some embodiments, the transgene encoding a disorder-related polypeptide may be codon-optimized. In some embodiments, the transgene encoding a disorder-related polypeptide may be codon optimized for expression in a particular cell, such as a eukaryotic cell. Eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon usage tables are readily available, for example, at the “Codon Usage Database”, and these tables can be adapted in a number of ways (see, e.g., Nakamura, Y. et al. (2000) Nucleic Acids Res. 28:292). Computer algorithms for codon optimizing a particular sequence for expression in a particular host cell are also available, such as Gene Forge (Aptagen; Jacobus, Pa.), DNA2.0, GeneArt (GA) or Genscript (GS) and a GS algorithm combined with reduction in CpG content. In some embodiments, a transgene encoding the disorder-related polypeptide is codon optimized using the GA algorithm.

In some embodiments, the expression cassette further comprises an intron. A variety of introns for use in the disclosure are known to those of skill in the art, and include the MVM intron, the F IX truncated intron 1, the B-globin SD/immunoglobin heavy chain SA, the adenovirus SD/immunoglobin SA, the SV40 late SD/SA (19S/16S), and the hybrid adenovirus SD/IgG SA. (Wu et al. 2008, Kurachi et al., 1995, Choi et al. 2014, Wong et al., 1985, Yew et al. 1997, Huang and Gorman (1990). In some embodiments, the intron is a chicken β-actin (CBA)/rabbit β-globin hybrid intron. In some embodiments, intron is a chicken β-actin (CBA)/rabbit β-globin hybrid promoter and intron where all the ATG sites are removed to minimize false translation start sites. In some embodiments the intron is an MVM intron, a FIX truncated intron 1, a β-globin SD/immunoglobin heavy chain SA, an adenovirus SD/immunoglobin SA, a SV40 late SD/SA (19S/16S), or a hybrid adenovirus SD/IgG SA. In some embodiments, the intron is a chicken β-actin (CBA)/rabbit β-globin hybrid intron.

In some embodiments, the expression cassette further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a synthetic polyadenylation signal as described in Levitt, N et al. (1989), Genes Develop. 3:1019-1025.

In some embodiments, the expression cassette comprises a stuffer nucleic acid. In some embodiments, the stuffer nucleic acid may comprise a sequence that encodes a reporter polypeptide. As will be appreciated by those of skill in the art, the stuffer nucleic acid may be located in a variety of regions within the nucleic, and may be comprised of a continuous sequence (e.g., a single stuffer nucleic acid in a single location) or multiple sequences (e.g., more than one stuffer nucleic acid in more than one location (e.g., 2 locations, 3 locations, etc.) within the nucleic acid. In some embodiments, the stuffer nucleic acid may be located downstream of the transgene encoding the disorder-related polypeptide. In some embodiments, the stuffer nucleic acid may be located upstream of the transgene encoding the disorder-related polypeptide (e.g., between the promoter and the transgene). As will also be appreciated by those of skill in the art a variety of nucleic acids may be used as a stuffer nucleic acid. In some embodiments, the stuffer nucleic acid comprises all or a portion of a human alpha-1-antitrypsin (AAT) stuffer sequence or a C16 P1 chromosome 16 P1 clone (human C16) stuffer sequence. In some embodiments, the stuffer sequence comprises all or a portion of a gene. For example, the stuffer sequence may comprise a portion of the human AAT sequence. One skilled in the art would recognize that different portions of a gene (e.g., the human AAT sequence) can be used as a stuffer fragment. For example, the stuffer fragment may be from the 5′ end of the gene, the 3′ end of the gene, the middle of a gene, a non-coding portion of the gene (e.g., an intron), a coding region of the gene (e.g., an exon), or a mixture of non-coding and coding portions of a gene. One skilled in the art would also recognize that all or a portion of stuffer sequence may be used as a stuffer sequence. In some embodiments, the stuffer sequence is modified to remove internal ATG codons.

In some embodiments, the expression cassette is incorporated into a vector. In some embodiments, the expression cassette is incorporated into a viral vector. In some embodiments, the viral vector is a rAAV vector as described herein.

### Vectors and Viral Particles

In certain aspects, the expression cassette for expressing a disorder-related polypeptide or RNAi molecule is contained in a vector. In some embodiments, the present disclosure contemplates the use of a recombinant viral genome for introduction of nucleic acid sequences encoding the disorder-related polypeptide or RNAi molecule for packaging into a viral particle, e.g., a viral particle described below. The recombinant viral genome may include any element to establish the expression of the disorder-related polypeptide, for example, a promoter, an ITR, a ribosome binding element, terminator, enhancer, selection marker, intron, polyA signal, and/or origin of replication. Exemplary viral genome elements and delivery methods for viral particles are described in greater detail below.

### Non-viral Delivery Systems

Conventional non-viral gene transfer methods may also be used to introduce nucleic acids into cells or target tissues. Non-viral vector delivery systems include DNA plasmids, naked nucleic acid, and nucleic acid complexed to a delivery system. For example, the vector may be complexed to a lipid (e.g., a cationic or neutral lipid), a liposome, a polycation, a nanoparticle, or an agent that enhances the cellular uptake of nucleic acid. The vector may be complexed to an agent suitable for any of the delivery methods described herein. In some embodiments, the nucleic acid comprises one or more viral ITRs (e.g., AAV ITRs).

### Viral Particles

In some embodiments, the vector comprising the expression cassette for expressing a disorder-related polypeptide or RNAi molecule is a recombinant viral vector. Some examples of recombinant viral vectors comprise AAV, lentivirus or adenovirus. In one aspect, the viral vector is a recombinant adeno-associated virus (rAAV) vector. In some embodiments, the expression cassette for expressing a disorder-related polypeptide or RNAi molecule may be flanked by one or more AAV inverted terminal repeat (ITR) sequences. In some embodiments, the viral particle is a recombinant AAV particle comprising an expression cassette for expressing a disorder-related polypeptide or RNAi molecule is flanked by one or two ITRs. In some embodiments, the expression cassette for expressing a disorder-related polypeptide or RNAi molecule is flanked by two AAV ITRs.

In some embodiments, the expression cassette for expressing a disorder-related polypeptide or RNAi molecule of the present disclosure comprises operatively linked components configured for transcription. In some embodiments, the expression cassette comprises control sequences further comprising transcription initiation, termination sequences, or a combination thereof. The expression cassette may be flanked on the 5′ and 3′ end by at least one functional AAV ITR sequence. By “functional AAV ITR sequences” it is meant that the ITR sequences function as intended for the rescue, replication and packaging of the AAV virion. See Davidson et al., PNAS, 2000, 97 (7) 3428-32; Passini et al., J. Virol., 2003, 77 (12): 7034-40; and Pechan et al., Gene Ther., 2009, 16:10-16, all of which are incorporated herein in their entirety by reference. For practicing some aspects of the disclosure, the recombinant vectors may comprise at least some or all of the sequences of AAV essential for encapsidation and the physical structures for infection by the rAAV. AAV ITRs for use in the vectors of the disclosure need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99 (18): 11854-6; Gao et al., PNAS, 2003, 100 (10): 6081-6; and Bossis et al., J. Virol., 2003, 77 (12): 6799-810.

Use of any AAV serotype is considered within the scope of the present disclosure. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, a goat AAV, bovine AAV, or mouse AAV ITRs or the like. In some embodiments, the nucleic acid in the AAV comprises an ITR of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV or the like. In certain embodiments, the AAV ITRs are AAV2 ITRs.

In some embodiments, a vector may include a stuffer nucleic acid. In some embodiments, the stuffer nucleic acid may encode a green fluorescent protein (GFP). In some embodiments, the stuffer nucleic acid may be located 3′ to expression cassette for expressing a disorder-related polypeptide of the present disclosure.

In some embodiments, the disclosure provides viral particles comprising a single-stranded genome. In some aspects, the disclosure provides viral particles comprising a recombinant self-complementing genome. In some embodiments, the vector is a self-complementary vector. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the disclosure provides an AAV viral particle comprising an AAV genome, wherein the rAAV genome comprises a first heterologous polynucleotide sequence (e.g., the coding strand of the disorder-related polypeptide of the disclosure) and a second heterologous polynucleotide sequence (e.g., the noncoding or antisense strand of the disorder-related polypeptide or RNAi molecule) wherein the first heterologous polynucleotide sequence can form intrastrand base pairs with the second polynucleotide sequence along most or all of its length.

In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in siRNA molecules. In some embodiments, the first heterologous polynucleotide sequence and a second heterologous polynucleotide sequence are linked by a mutated ITR (e.g., the right ITR). The mutated ITR may comprise a deletion of the D region comprising the terminal resolution sequence. As a result, on replicating an AAV viral genome, the rep proteins may not cleave the viral genome at the mutated ITR and as such, a recombinant viral genome comprising the following in 5′ to 3′ order may be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the first heterologous nucleic acid sequence and a second heterologous nucleic acid sequence are linked by a mutated ITR (e.g., the right ITR). In some embodiments, the ITR comprises the polynucleotide sequence 5′-CACTCCCTCTCTGCGCGCT CGCTCGCTCACTGAGGCCGGGCGACCAAAGGTCGCCCACGCCCGGGCTTTGCCCGG GCG-3′ (SEQ ID NO:15). The mutated ITR may comprise a deletion of the D region comprising the terminal resolution sequence. As a result, on replicating an AAV viral genome, the rep proteins may not cleave the viral genome at the mutated ITR and as such, a recombinant viral genome comprising the following in 5′ to 3′ order may be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the vector is encapsidated in a viral particle. In some embodiments, the viral particle is a recombinant AAV viral particle comprising a recombinant AAV vector. Different AAV serotypes may be used to optimize transduction of particular target cells or to target specific cell types within a particular target tissue (e.g., brain or spinal). A rAAV particle can comprise viral proteins and viral nucleic acids of the same serotype or a mixed serotype. For example, in some embodiments a rAAV particle can comprise modified AAV.SAN006 capsid proteins and at least one AAV2 ITR or it can comprise modified AAV.SAN006 capsid proteins and at least one AAV1 ITR. Any combination of AAV serotypes for production of a rAAV particle may be provided herein as if each combination had been expressly stated herein.

The capsid (e.g., SAN006) or the rAAV viral particle is known to include three capsid proteins: VP1, VP2, and VP3. These proteins contain significant amounts of overlapping amino acid sequence and unique N-terminal sequences. An AAV9 capsid includes 60 subunits arranged by icosahedral symmetry. AAV9 includes VP1 (SEQ ID NO: 9), VP2 (SEQ ID NO: 13), and VP3 (SEQ ID NO: 14), capsid proteins in a ratio of about 5:5:50. The VP proteins of AAV9 are products of the structural protein-encoding open reading frame of the genome, designated cap, VP1 (˜82 kDa) and VP2 (˜73 kDa), which are the minor capsid proteins, and VP3 (˜61 kDa), the major capsid protein. Due to the utilization of both alternative splicing and leaky scanning, when expressed, the individual VPs share a C terminus that encompasses the entire VP3, while VP1 and VP2 are N-terminal VP3 extensions. VP1 and VP2 share a region of ˜73 amino acids amino acids which is extended by an additional ˜ 137 amino acids in VP1, designated the VP1 unique region (VPlu). See Penzes et al., (2021), Journal of Virology 95 (19) e0084321. In some embodiments of the modified AAV9 capsid proteins disclosed herein, the targeting peptide (e.g., SEQ ID NO: 10) is incorporated into VP1. In some embodiments of the modified AAV9 capsid proteins disclosed herein, the targeting peptide (e.g., SEQ ID NO:10) is incorporated into VP2. In some embodiments of the modified AAV9 capsid proteins disclosed herein, the targeting peptide (e.g., SEQ ID NO:10) is incorporated into VP3. In some embodiments of the modified AAV9 capsid proteins disclosed herein, the targeting peptide (e.g., SEQ ID NO:10) is incorporated into VP1, VP2 and VP3.

In some embodiments, the disclosure provides rAAV particles for intra-CSF administration of a disorder-related polypeptide that may comprise a modified AAV9 capsid protein comprising a targeting peptide that may target (e.g., direct) the rAAV particles to a particular tissue (e.g., the brain). In particular embodiments, the targeting peptide of the modified AAV9 capsids are inserted after residue 588 of the AAV9 structural protein. In some embodiments, the targeting peptide has SEQ ID NO: 10. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 11. In some embodiments, the full modified AAV9 capsid structural protein has SEQ ID NO: 12. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 12, wherein the modified AAV9 structural capsid comprises the targeting peptide of SEQ ID NO: 10.

### Production of AAV Particles

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997) J. Virology 71 (11): 8780-8789) and baculovirus-AAV hybrids (Urabe, M. et al., (2002) Human Gene Therapy 13 (16): 1935-1943; Kotin, R. (2011) Hum Mol Genet. 20 (R1): R2-R6). rAAV production cultures for the production of rAAV virus particles all require; 1) suitable host cells, 2) suitable helper virus function, 3) AAV rep and cap genes and gene products; 4) a nucleic acid (such as a therapeutic nucleic acid) flanked by at least one AAV ITR sequences (e.g., an AAV genome encoding a peptide of interest); and 5) suitable media and media components to support rAAV production. In some embodiments, the suitable host cell is a primate host cell. In some embodiments, the suitable host cell is a human-derived cell lines such as HeLa, A549, 293, or Perc.6 cells. In some embodiments, the suitable helper virus function is provided by wild-type or mutant adenovirus (such as temperature sensitive adenovirus), herpes virus (HSV), baculovirus, or a plasmid construct providing helper functions. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

One method for producing rAAV particles is the triple transfection method. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the rAAV particle was produced by triple transfection of a nucleic acid encoding the rAAV vector, a nucleic acid encoding AAV rep and cap, and a nucleic acid encoding AAV helper virus functions into a host cell, wherein the transfection of the nucleic acids to the host cells generates a host cell capable of producing rAAV particles.

In some embodiments, rAAV particles may be produced by a producer cell line method (see Martin et al., (2013) Human Gene Therapy Methods 24:253-269; U.S. PG Pub. No. US2004/0224411; and Liu, X. L. et al. (SAN006) Gene Ther. 6:293-299). Briefly, a cell line (e.g., a HeLa, 293, A549, or Perc.6 cell line) may be stably transfected with a plasmid containing a rep gene, a capsid gene, and a vector genome comprising a promoter-heterologous nucleic acid sequence (e.g., a disorder-related polypeptide). Cell lines may be screened to select a lead clone for rAAV production, which may then be expanded to a production bioreactor and infected with a helper virus (e.g., an adenovirus or HSV) to initiate rAAV production. Virus may subsequently be harvested, adenovirus may be inactivated (e.g., by heat) and/or removed, and the rAAV particles may be purified. As such, in some embodiments, the rAAV particle was produced by a producer cell line comprising one or more of nucleic acid encoding the rAAV vector, a nucleic acid encoding AAV rep and cap, and a nucleic acid encoding AAV helper virus functions. As described herein, the producer cell line method may be advantageous for the production of rAAV particles with an oversized genome, as compared to the triple transfection method.

In some embodiments, the nucleic acid encoding AAV rep and cap genes and/or the rAAV genome are stably maintained in the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, a cell line stably transfected with a plasmid maintains the plasmid for multiple passages of the cell line (e.g., 5, 10, 20, 30, 40, 50 or more than 50 passages of the cell). For example, the plasmid(s) may replicate as the cell replicates, or the plasmid(s) may integrate into the cell genome. A variety of sequences that enable a plasmid to replicate autonomously in a cell (e.g., a human cell) have been identified (see, e.g., Krysan, P. J. et al. (1989) Mol. Cell Biol. 9:1026-1033). In some embodiments, the plasmid(s) may contain a selectable marker (e.g., an antibiotic resistance marker) that allows for selection of cells maintaining the plasmid. Selectable markers commonly used in mammalian cells include without limitation blasticidin, G418, hygromycin B, zeocin, puromycin, and derivatives thereof. Methods for introducing nucleic acids into a cell are known in the art and include without limitation viral transduction, cationic transfection (e.g., using a cationic polymer such as DEAE-dextran or a cationic lipid such as lipofectamine), calcium phosphate transfection, microinjection, particle bombardment, electroporation, and nanoparticle transfection (for more details, see e.g., Kim, T. K. and Eberwine, J. H. (2010) Anal. Bioanal. Chem. 397:3173-3178).

In some embodiments, the nucleic acid encoding AAV rep and cap genes and/or the rAAV genome are stably integrated into the genome of the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, the plasmid(s) may contain a selectable marker (e.g., an antibiotic resistance marker) that allows for selection of cells maintaining the plasmid. Methods for stable integration of nucleic acids into a variety of host cell lines are known in the art. For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A. C. et al. (2000) Proc. Natl. Acad. Sci. 97:5995-6000).

In some embodiments, the producer cell line is derived from a primate cell line (e.g., a non-human primate cell line, such as a Vero or FRhL-2 cell line). In some embodiments, the cell line is derived from a human cell line. In some embodiments, the producer cell line is derived from HeLa, 293, A549, or PERC.6® (Crucell) cells. For example, prior to introduction and/or stable maintenance/integration of nucleic acid encoding AAV rep and cap genes and/or the oversized rAAV genome into a cell line to generate a producer cell line, the cell line is a HeLa, 293, A549, or PERC.6® (Crucell) cell line, or a derivative thereof.

In some embodiments, the producer cell line is adapted for growth in suspension. As is known in the art, anchorage-dependent cells are typically not able to grow in suspension without a substrate, such as microcarrier beads. Adapting a cell line to grow in suspension may include, for example, growing the cell line in a spinner culture with a stirring paddle, using a culture medium that lacks calcium and magnesium ions to prevent clumping (and optionally an antifoaming agent), using a culture vessel coated with a siliconizing compound, and selecting cells in the culture (rather than in large clumps or on the sides of the vessel) at each passage. For further description, see, e.g., ATCC frequently asked questions document (available at www.atcc.org/Global/FAQs/9/1/Adapting % 20a %20monolayer %20cell %20line %20 to %20suspen sion-40.aspx) and references cited therein.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a heterologous nucleic acid as described herein flanked by at least one AAV ITR, and (iii) an AAV helper function; and (b) recovering the rAAV particles produced by the host cell. In some embodiments, said at least one AAV ITR is selected from the group consisting of AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV serotype ITRs or the like. For example, in some embodiments, the AAV serotype is AAV1, AAV2, AAV5, AAV6, AAV7, AAV8, AAVrh8, AAVrh8R, AAV9, AAV10, or AAVrh10. In certain embodiments, the nucleic acid in the AAV comprises an AAV2 ITR. In some embodiments, said encapsidation protein is selected from the group consisting of AAV1, AAV2, AAV3, AAV4, AAV5, AAV6, AAV7, AAV8, AAVrh8, AAVrh8R, AAV9, AAVSAN006. AAV10, AAVrh10, AAV11, AAV12, AAV2R471A, AAV2/2-7m8, AAV DJ, AAV2 N587A, AAV2 E548A, AAV2 N708A, AAV V708K, goat AAV, AAV1/AAV2 chimeric, bovine AAV, mouse AAV capsid, rAAV2/HBoV1 serotype, AAV-XL32, or AAV-XL32.1 capsid proteins or mutants thereof. In some embodiments, the encapsidation protein is an AAV8 capsid protein. In some embodiments, the rAAV particles comprise an AAV9 capsid and a recombinant genome comprising AAV2 ITRs, and nucleic acid encoding a therapeutic transgene/nucleic acid (e.g., an expression cassette for expressing a disorder-related polypeptide). In some embodiments, the rAAV particles comprise an AAV.SAN006 capsid and a recombinant genome comprising AAV2 ITRs, and nucleic acid encoding a therapeutic transgene/nucleic acid (e.g., an expression cassette for expressing a disorder-related polypeptide).

Suitable rAAV production culture media of the present disclosure may be supplemented with serum or serum-derived recombinant proteins at a level of 0.5%-20% (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2 μm Filter Opticap XL10 Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

### Methods of Treatment

Certain aspects of the present disclosure relate to methods of treating various neurodegenerative diseases in an individual in need thereof. In some embodiments, the disclosure provides methods of treating a neurodegenerative disease by administering an effective amount of a viral particle of the disclosure for expressing a disorder-related polypeptide or RNAi molecule. The viral particles may be administered through various routes. In some embodiments, the viral particles are capable of expressing a polypeptide associated with a neurodegenerative disorder. In other embodiments, the viral particles are capable of expressing an RNAi molecule capable of reducing or eliminating the expression of a particular polypeptide implicated in a neurodegenerative disorder as set forth herein. In some embodiments, the RNAi molecule is an artificial miRNA.

In some embodiments, the administration of the viral particle includes direct spinal cord injection and/or intracerebral administration. In some embodiments, the administration is at a site selected from the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration comprises intracerebroventricular injection into at least one cerebral lateral ventricle. In some embodiments, the administration comprises intrathecal injection in the cervical, thoracic, and/or lumbar region. In some embodiments, the administration comprises intrastriatal injection. In some embodiments, the administration comprises intrathalamic injection.

In some embodiments of the above aspects, the rAAV is administered via direct injection into the spinal cord, via intrathecal injection, or via intracisternal injection. In some embodiments, the rAAV is administered to more than one location of the spinal cord or cisterna magna. In some embodiments, the rAAV is administered to more than one location of the spinal cord. In some embodiments, the rAAV is administered to one or more of a lumbar subarachnoid space, thoracic subarachnoid space and a cervical subarachnoid space of the spinal cord. In some embodiments, the rAAV is administered to the cisterna magna.

An effective amount of rAAV (in some embodiments in the form of particles) may be administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells of the desired tissue type, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. One or more of any of the routes of administration described herein may be used. In some embodiments, multiple vectors may be used to treat the human.

Methods to identify cells transduced by AAV viral particles are known in the art; for example, immunohistochemistry or the use of a marker such as enhanced green fluorescent protein can be used to detect transduction of viral particles; for example viral particles comprising a rAAV capsid with one or more substitutions of amino acids.

In some embodiments, an effective amount of rAAV particles is administered to more than one location simultaneously or sequentially. In other embodiments, an effective amount of rAAV particles is administered to a single location more than once (e.g., repeated). In some embodiments, multiple injections of rAAV viral particles are no more than one hour, two hours, three hours, four hours, five hours, six hours, nine hours, twelve hours or 24 hours apart.

In some embodiments, the disclosure provides a method for treating a human with a neurodegenerative disorder by administering an effective amount of a pharmaceutical composition comprising a recombinant viral vector encoding a disorder-related polypeptide or RNAi molecule. In some embodiments, the pharmaceutical composition comprises one or more pharmaceutically acceptable excipients.

In some embodiments, the methods comprise administering an effective amount of a pharmaceutical composition comprising a recombinant viral vector encoding a disorder-related polypeptide of the present disclosure to treat a neurodegenerative disorder in an individual in need thereof. In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some embodiments, the viral titer of the viral particles (e.g., rAAV particles) is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the viral particles (e.g., rAAV particles) is about any of 5×10¹² to 10×10¹², 10 ×10¹² to 25×10¹², or 25×10¹² to 50×10¹² genome copies/mL. In some embodiments, the viral titer of the viral particles (e.g., rAAV particles) is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the viral particles (e.g., rAAV particles) is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the viral particles (e.g., rAAV particles) is about any of 5×10⁹ to 10×10⁹, 10×10⁹ to 15×10⁹, 15×10⁹ to 25×10⁹, or 25×10⁹ to 50×10⁹ transducing units/mL. In some embodiments, the viral titer of the viral particles (e.g., rAAV particles) is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the viral particles (e.g., rAAV particles) is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the viral particles (e.g., rAAV particles) is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL. In some embodiments, the viral particles are rAAV particles. In some embodiments, the rAAV particles comprise an AAV.SAN006 capsid protein.

In some embodiments, the dose of viral particles administered to the individual is at least about any of 1×10⁸ to about 6×10¹³ genome copies/kg of body weight. In some embodiments, the dose of viral particles administered to the individual is about any of 1×10⁸ to about 6×10¹³ genome copies/kg of body weight. In some embodiments, the dose of viral particles administered to the individual is about any of 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 13×10¹², 4×10¹², 5×10¹², 6×10¹², 7×10¹², 8×10¹², 9×10¹², or 1×10¹³ genome copies/kg of body weight.

In some embodiments, the total amount of viral particles administered to the individual is at least about any of 1×10⁹ to about 1×10¹⁴ genome copies. In some embodiments, the total amount of viral particles administered to the individual is about any of 1×10⁹ to about 1×10¹⁴ genome copies. In some embodiments, the total amount of viral particles administered to the individual is about any of 1×10¹¹, 2×10¹¹, 3×10¹¹, 4×10¹¹, 5×10¹¹, 6×10¹¹, 7×10¹¹,8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 13×10¹³, 4×10¹³, 5×10¹³, 6×10¹³, 7×10¹³, 8×10¹³, 9×10¹³, or 1×10¹⁴ genome copies.

Compositions of the disclosure (e.g., recombinant viral particles comprising a vector encoding a disorder-related polypeptide of the present disclosure) can be used either alone or in combination with one or more additional therapeutic agents for treating a neurodegenerative disorder. The interval between sequential administration can be in terms of at least (or, alternatively, less than) minutes, hours, or days.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. In some embodiments, multiple vectors may be used to treat the mammal (e.g., a human).

In some embodiments, a rAAV composition of the present disclosure may be used for administration to a human. In some embodiments, a rAAV composition of the present disclosure may be used for pediatric administration. In some embodiments, an effective amount of rAAV (in some embodiments in the form of particles) is administered to a patient that is less than one month, less than two months, less than three months, less than four months, less than five months, less than six months, less than seven months, less than eight months, less than nine months, less than ten months, less than eleven months, less than one year, less than 13 months, less than 14 months, less than 15 months, less than 16 months, less than 17 months, less than 18 months, less than 19 months, less than 20 months, less than 21 months, less than 22 months, less than two years, less than three years old, less than five years old or less than seven years old.

In some embodiments, a rAAV composition of the present disclosure may be used for administration to a young adult. In some embodiments, an effective amount of rAAV (in some embodiments in the form of particles) is administered to a patient that is less than 12 years old, less than 13 years old, less than 14 years old, less than 15 years old, less than 16 years old, less than 17 years old, less than 18 years old, less than 19 years old, less than 20 years old, less than 21 years old, less than 22 years old, less than 23 years old, less than 24 years old, or less than 25 years old.

### Methods for Treating Parkinson's Disease and Associated Disorders

Of the most common neurodegenerative disorders are Parkinson's Disease (PD) and its associated disorders. PD and associated disorders are brain disorders that cause uncontrollable movement and reduced balance and coordination. PD and related movement disorders can include lewy body dementias (LBD) and multiple system atrophy (MSA). These disorders are characterized by neurodegeneration of basal ganglia, brainstem, cerebellum, and cerebral cortex. PD and its associated disorders are related to dysfunction of any of several disease associated genes, including, but not limited to: ATP13A2, PLA2G6, VPS35, DJ1, GBA1, SNCA, PINK1, PARKIN or LRRK2. For example, the ATP13A2 gene encodes a member of the P5 subfamily of ATPases which transports inorganic cations as well as other substrates. Mutations in this gene are associated with Kufor-Rakeb syndrome (KRS), also referred to as Parkinson disease 9. KRS is a very rare form of inherited juvenile-onset Parkinson's disease. As an additional example, the SNCA gene expresses Synucleins (e.g., small, soluble proteins) in the brain. Defects in the SNCA gene have been implicated in the pathogenesis of Parkinson disease.

Certain aspects of the present disclosure relate to methods of treating Parkinson's disease (PD) and associated disorders in an individual in need thereof. In some embodiments, the disclosure provides methods of treating PD and/or associated disorders by administering an effective amount of viral particles as disclosed herein. In some embodiments, the disclosure provides methods of treating PD and/or associated disorders administering an effective amount of viral particles comprising expression cassette for expressing a disorder-related polypeptide of the present disclosure. In some embodiments, the disorder-related polypeptide is a wild type disorder-related polypeptide.

In some embodiments, the method for treating PD and/or associated disorders may comprise delivering a viral particle expressing a transgene for modulating the function or levels of at least one target. In some embodiments, the method may comprise delivering an expression cassette expressing a transgene for modulating the function or levels of at least one target intracellularly or extracellularly. In some embodiments, wherein the method comprises delivering an expression cassette intracellularly, the method may comprise delivering a miRNA (e.g., artificial miRNA) targeting SNCA to lower expression. In some embodiments, the method may comprise intracellular delivery of a viral particle comprising an artificial miRNA configured to target SNCA and lower expression. In some embodiments, the method may comprise extracellular delivery of a viral particle comprising an expression cassette configured for expression of an antibody fragment for secretion and binding to a target of interest either intra- or extracellularly.

In some embodiments, expression of the disorder-related polypeptide is under control of a promoter. In some embodiments, the expression cassette is under the control of a promoter. In some embodiments, the transgene may be under the control of a promoter. In some embodiments, the promoter may comprise a ubiquitous promoter or a neuron-specific promoter. In some embodiments, the ubiquitous promoter may comprise CBA, CAG, CAGG, CMV, H1, U6, or 7SK. In some embodiments, the neuron-specific promoter may comprise SYN1, NSE, CAMKII, or TUBB3. In some instances, the promoter (e.g., promoter activity) may be regulated by an exogenous small molecule.

In some embodiments, the expression cassette may comprise at least one transgene. In some embodiments, at least one transgene (e.g., gene) may comprise ATP13A2, PLA2G6, VPS35, DJ1, GBA1, SNCA, PINK1, PARKIN or LRRK2. In some instances, transgene expression may be regulated by an exogenous small molecule.

In some instances, the expression cassette may comprise at least one additional nucleotide sequence. In some embodiments, at least one additional nucleotide sequence may be configured to improve RNA stability. In some embodiments, at least one additional nucleotide sequence configured to improve RNA stability may comprise WPRE. In some embodiments, at least one additional nucleotide sequence may be configured to detarget expression from liver and/or DRG.

### Methods for Treating Alzheimer's Disease and Associated Disorders

Another common neurodegenerative disorder is Alzheimer's Disease (AD). In fact, AD and associated disorders comprise the most common form of dementia. Two main neuropathological lesions of AD are amyloid plaques and neurofibrillary tangles (NFTs). Amyloid plaques are composed of amyloid β (Aβ) peptides, which are cleaved from the Amyloid Precursor Protein (APP). NFTs, present in the brain of AD and related neurodegenerative disease patients, are constituted of tau proteins in hyperphosphorylated and aggregated form. Disorders associated with AD (e.g., related neurodegenerative disorders) are cerebral amyloid angiopathy (CAA), progressive supranuclear palsy (PSP), corticobasal degeneration (CBD), frontotemporal dementia with parkinsonism-17 (FTDP-17), Pick's Disease (PiD), argyrophilic grain disease (AGD), and globular glial tauopathy (GGT). AD and CAA are characterized by intracellular aggregation of hyperphosphorylated tau protein with extracellular aggregation of amyloid beta (AB) plaques. Whereas, the associated disorders including PSP, CBD, FTDP-17, PiD, GGT, and AGD are characterized by intracellular aggregation of hyperphosphorylated tau protein without extracellular aggregation of amyloid beta (AB) plaques. These pathological protein aggregates can cause synaptic dysfunction, neuroinflammation, and neurodegeneration in cerebral cortex, cerebellum, basal ganglia, midbrain, and brainstem. These processes can be related to dysfunction of any of several disease-associated genes, including, but not limited to: MAPT, BIN1, APP, PS1, APOE, CD33, and/or TREM2

Certain aspects of the present disclosure relate to methods of treating Alzheimer's Disease (AD) and/or associated disorders in an individual in need thereof. In some embodiments, the disclosure provides methods of treating AD and/or associated disorders by administering an effective amount of a viral particle of the disclosure. In some embodiments, the disclosure provides methods of treating AD and/or associated disorders by administering an effective amount of a viral particle for expressing a disorder-related polypeptide or RNAi molecule.

In some embodiments, the method comprises delivering a viral particle comprising am expression cassette configured to target tau pathology in AD and/or associated disorders. In some embodiments, the method comprises delivering a viral particle encoding an artificial miRNA. In some embodiments, the method comprises delivering a viral particle encoding an antisense oligonucleotide (ASO). In some embodiments, the method may comprise delivering a viral particle comprising an expression cassette configured to target MAPT or BIN1 mRNA. In some embodiments, the method may comprise delivering a viral particle comprising an expression cassette configured to target a vectorized antibody targeting tau or Bin1 protein. In some embodiments, the disorder-related polypeptide comprises the vectorized antibody targeting tau. In some embodiments, the disorder-related polypeptide comprises the vectorized antibody targeting the Bin1 protein.

In some embodiments, the expression cassette configured to target tau pathology in AD and/or associated disorders, may be under control of a promoter. In some embodiments, the promoter may comprise a ubiquitous or neuron-specific promoter. In some embodiments, the ubiquitous promoter may comprise CBA, CAG, CAGG, CMV, H1, U6 or 7SK. In some embodiments, the neuron-specific promoter may comprise SYN1, NSE, CAMKII, or TUBB3. In some instances, the promoter may be regulated by an exogenous small molecule.

In some embodiments, the method may comprise delivering a viral particle comprising an expression cassette configured to target Aβ pathology in AD and/or associated disorders. In some embodiments, the method comprises delivering a viral particle comprising an expression cassette (e.g., AAV genome) encoding an artificial miRNA. In some embodiments, the method comprises delivering a viral particle comprising an expression cassette (e.g., AAV genome) encoding an antisense oligonucleotide (ASO). In some embodiments, the method comprises delivering a viral particle comprising an expression cassette configured to target APP or PS1 gene. In some embodiments, the method comprises delivering a viral particle comprising an expression cassette configured to target a vectorized antibody targeting AB.

In some embodiments, the expression cassette configured to target AB pathology in AD and/or associated disorders, may be under control of a promoter. In some embodiments, the promoter may comprise a ubiquitous or neuron-specific promoter. In some embodiments, the ubiquitous promoter may comprise CBA, CAG, CAGG, CMV, H1, U6 or 7SK. In some embodiments, the neuron-specific promoter may comprise SYN1, NSE, CAMKII, or TUBB3. In some instances, promoter may be regulated by an exogenous small molecule.

In some embodiments, the method for treating AD and/or associated disorders, wherein patients comprise the APOE4 risk allele, the method may comprise delivering an expression cassette (e.g., AAV genome) encoding an ASO targeting APOE4 and/or encoding the protective allele APOE2.

In some embodiments, the method for treating AD and/or associated disorders may be configured to target innate immune pathways contributing to neuroinflammation. In such methods, the method may comprise delivering an expression cassette (e.g., AAV genome) encoding an artificial miRNA or vectorized antibody targeting CD33 or encoding the soluble form of TREM2.

In some embodiments, wherein the method is configured to target innate immune pathways contributing to neuroinflammation, the expression cassette may be under the control of a promoter. In such methods, AAV-encoded therapeutics may be expressed under control of ubiquitous or neuron-specific promoters. In such embodiments, the ubiquitous promoter may comprise CBA, CAG, CAGG, CMV, H1, U6, or 7SK. In such embodiments, the neuron-specific promoter may comprise SYN1, NSE, CAMKII, or TUBB3.

In some instances, the viral particle may encode at least one nucleotide sequence. In some embodiments, at least one nucleotide sequence may be configured to improve RNA stability. In some embodiments, wherein at least one nucleotide sequence is configured to improve RNA stability, at least one of the nucleotide sequences may comprise WPRE. In some embodiments, at least one nucleotide sequence may be configured to target expression from liver and/or DRG.

In some embodiments, the expression cassette may comprise at least one transgene. In some embodiments, at least one transgene may comprise MAPT, BIN1, APP, PS1, APOE, CD33, TREM2, or a combination thereof. In some instances, the transgene expression may be regulated by an exogenous small molecule.

Methods for treating Metachromatic Leukodystrophy

Metachromatic leukodystrophy (MLD) is an autosomal recessive neurodegenerative disorder caused by mutations in the enzyme arylsulfatase A (ARSA). Reduced levels of ARSA activity result in toxic accumulation of sulfatide characterized by degeneration of myelin-forming cells (oligodendrocytes and Schwann cells) in the central and peripheral nervous system. This results in demyelination, dysfunction, degeneration of neurons and neuroinflammation (astrocytosis, microglial activation). Clinical manifestations are primarily in the nervous system, resulting in intellectual disability, emotional and behavioral problems, loss of motor skills (moving, speaking, swallowing), poor muscle function and paralysis, blindness, hearing loss and seizures. The current treatment for MLD involves autologous hematopoietic stem cell transplantation (HSCT) involving allogenic bone marrow transplant.

Certain aspects of the present disclosure relate to methods of treating MLD and/or increasing levels of an ARSA polypeptide in an individual in need thereof. In some embodiments, the disclosure provides methods of treating MLD by administering an effective amount of a viral particle of the disclosure for expressing an ARSA polypeptide. In some embodiments, the ARSA polypeptide is a wild type ARSA polypeptide.

In some embodiments, the method may provide for treating a human with MLD by administering an effective amount of a pharmaceutical composition comprising a recombinant viral vector encoding an ARSA polypeptide of the present disclosure. In some embodiments, the pharmaceutical composition comprises one or more pharmaceutically acceptable excipients.

Methods for treating Adrenoleukodystrophy and Adrenomyeloneuropathy

Certain aspects of the present disclosure relate to methods of treating Adrenoleukodystrophy (ALD) and Adrenomyeloneuropathy (ALM) in an individual in need thereof. In some embodiments, the disclosure provides methods of treating ALD and/or ALM by administering an effective amount of a viral particle of the disclosure. In some embodiments, the disclosure provides methods of treating a ALD and/or ALM by administering an effective amount of a viral particle comprising an expression cassette for expressing a disorder-related polypeptide or RNAi molecule. In some embodiments, the disorder-related polypeptide may comprise human ABCD1 polypeptide. In some embodiments, the expression cassette of the viral particle may comprise a transgene for encoding human ABCD1 polypeptide.

In some embodiments, expression of the disorder-related polypeptide is under control of a promoter. In some embodiments, the promoter may comprise a ubiquitous, neuron-specific, or astrocyte-specific promoter.

In some instances, the expression cassette of the viral particle may comprise at least one additional nucleotide sequence. In some embodiments, the at least one additional nucleotide sequence may be for improving RNA stability. In some embodiments, the at least one additional nucleotide sequence may be for improving RNA stability may comprise WPRE. In some embodiments, the at least one additional nucleotide sequence may be for detargeting expression from liver and/or DRG.

In some instances, the expression cassette comprising the transgene encoding for the ABCD1 polypeptide may be combined with an artificial miRNA. In alternative embodiments, two viral particle can be delivered to the patient, one encoding the ABCD1 polypeptide and the other encoding the artificial miRNA. In some embodiments, the artificial miRNA may be configured for targeting ELOVL1. In some embodiments, the artificial miRNA may be configured for targeting ELOVL1 to reduce expression of ELOVL1.

In some instances, promoter activity and/or transgene expression may be regulated by an exogenous small molecule.

The expression cassette (e.g., expression cassette delivered in a rAAV particle) for expressing a disorder-related polypeptide may be administered through various routes as provided in the present disclosure.

Methods for Treating Amyotrophic Lateral Sclerosis and/or Frontotemporal Dementia

Amyotrophic lateral sclerosis (ALS) and frontotemporal dementia (FTD) are two neurodegenerative diseases with considerable genetic overlap. ALS and FTD together are a heterogeneous group of disorders characterized by degeneration of the cerebral cortex and/or the spinal cord. This degeneration is related to dysfunction of one of several disease-causing genes, including, but not limited to: SOD1, TARDBP, C9Orf72, FUS, GRN, VCP, TBK1, RIPK1, MAPT, NEK1, SQSTM1, CHCHD10. Studies involving these genes has led to the identification of neurodegeneration pathways such as autophagy, RNA regulation, and vesicle and inclusion formation.

Certain aspects of the present disclosure relate to methods of treating Amyotrophic Lateral Sclerosis (ALS) and/or Frontotemporal Dementia (FTD) in an individual in need thereof. In some embodiments, the method of treating ALS and/or FTD comprises administering an effective amount of a viral particle of the disclosure. In some embodiments, the expression cassette of the viral particle may comprise a transgene. In some embodiments, the expression cassette may be configured for encoding an artificial miRNA. In some embodiments, the expression cassette may be configured for encoding a vectorized antibody. In some embodiments, the vectorized antibody may be configured to target genes comprising SOD1, TARDBP, C9orf72 repeat expansion, FUS, RIPK1, or MAPT genes.

In some embodiments, the expression cassette may be under control of a promoter. In some embodiments, the promoter may comprise a ubiquitous or neuron-specific promoter. In some embodiments, the ubiquitous promoter may comprise CBA, CAG, CAGG, CMV, H1, U6 or 7SK. In some embodiments, the neuron-specific promoter may comprise SYN1, NSE, CAMKII, or TUBB3. In some instances, promoter may be regulated by an exogenous small molecule.

In some embodiments, the method may comprise regulating transgene expression by an exogenous small molecule.

In some instances, expression cassettes (e.g., AAV genomes) may encode for functional versions of a targeted gene.

In some instances, the expression cassette (e.g., AAV genome, genome, etc.) may encode artificial miRNA. In some embodiments, the artificial miRNA may be configured to target endogenous, affected gene, as well as nucleotide sequences to improve RNA stability. In some embodiments, the artificial miRNA (e.g., at least one additional nucleotide sequence) may comprise WPRE. In some embodiments, at least one additional nucleotide sequence may be configured to detarget expression from liver and/or DRG.

Methods for treating Neuropathic Pain

Neuropathic pain (NP) arises due to injury of the somatosensory nervous system. NP is characterized by dysfunction of dorsal root ganglia, spinal cord, and/or cerebral cortex. Often, NP is caused by nerve injury or trauma related to dysfunction of at least one of several genes, including, but not limited to: ADORA2A, CACNAIB, CACNA2D1, CACNA2D3, CACNA1H, CALCA, CALCB, GABBR1, GABBR2, GABRA1, GABRA2, GABRG1, GABRB3, GABRG2, GAD1, GAD2, HCN1, HCN2, HCN4, KCNA1, KCNA2, KCNB1, P2RX7, SCN9A, SCN10A, SCN11A, SLC12A2, SLC12A5, TRPA1, TRPV1. Patients with NP suffer not only from pain but also a high level of disability. Additionally, currently used therapeutics such as opioids can be highly addictive. Therefore, a cost-effective and non-addictive therapeutic is needed.

Certain aspects of the present disclosure relate to methods of treating Neuropathic Pain (NP) in an individual in need thereof. In some embodiments, the method of treating MP may comprise administering an effective amount of a viral particle of the disclosure. In some embodiments, the expression cassette may comprise a transgene. In some embodiments, the transgene (e.g., gene) may comprise ADORA2A, CACNAIB, CACNA2D1, CACNA2D3, CACNA1H, CALCA, CALCB, GABBR1, GABBR2, GABRA1, GABRA2, GABRG1, GABRB3, GABRG2, GAD1, GAD2, HCN1, HCN2, HCN4, KCNA1, KCNA2, KCNB1, P2RX7, SCN9A, SCN10A, SCN11A, SLC12A2, SLC12A5, TRPA1, or TRPV1.

In some embodiments, the expression cassette of the viral particle may be configured for encoding an artificial miRNA. In some embodiments, the rAAV particle may comprise an artificial RNA. In some embodiments, the expression cassette of the viral particle may be configured for encoding a target polypeptide. In some embodiments, the expression cassette of the viral particle may be configured for encoding a target polypeptide (e.g., disorder-related polypeptide). In some embodiments, the expression cassette of the viral particle may be configured for encoding the disorder-related polypeptide, wherein the disorder-related polypeptide may comprise ADORA2A, CACNA1B, CACNA2D1, CACNA2D3, CACNA1H, CALCA, CALCB, GABBR1, GABBR2, GABRA1, GABRA2, GABRG1, GABRB3, GABRG2, GAD1, GAD2, HCN1, HCN2, HCN4, KCNA1, KCNA2, KCNB1, P2RX7, SCN9A, SCN10A, SCN11A, SLC12A2, SLC12A5, TRPA1, TRPV1, or any combination thereof.

In some embodiments, the expression cassette of the viral particle may comprise at least one transgene for encoding at least one disorder-related polypeptide. In some embodiments, the expression cassette may comprise at least one transgene for encoding at least one polypeptide, wherein the at least one polypeptide may comprise ADORA2A, CACNAIB, CACNA2D1, CACNA2D3, CACNA1H, CALCA, CALCB, GABBR1, GABBR2, GABRA1, GABRA2, GABRG1, GABRB3, GABRG2, GAD1, GAD2, HCN1, HCN2, HCN4, KCNA1, KCNA2, KCNB1, P2RX7, SCN9A, SCN10A, SCN11A, SLC12A2, SLC12A5, TRPA1, TRPV1, or any combination thereof.

In some embodiments, the method may comprise delivering a viral particle encoding at least one artificial miRNA targeting ADORA2A, CACNAIB, CACNA2D1, CACNA2D3, CACNA1H, CALCA, CALCB, GABBR1, GABBR2, GABRA1, GABRA2, GABRG1, GABRB3, GABRG2, GAD1, GAD2, HCN1, HCN2, HCN4, KCNA1, KCNA2, KCNB1, P2RX7, SCN9A, SCN10A, SCN11A, SLC12A2, SLC12A5, TRPA1, TRPV1 or any combination thereof.

In some embodiments, the expression cassette may be under control of a promoter. In some embodiments, the promoter may comprise a ubiquitous or neuron-specific promoter. In some embodiments, the ubiquitous promoter may comprise CBA, CAG, CAGG, CMV, H1, U6 or 7SK. In some embodiments, the neuron-specific promoter may comprise SYN1, NSE, CAMKII, or TUBB3. In some instances, promoter may be regulated by an exogenous small molecule.

In some embodiments, the method may comprise regulating transgene expression by an exogenous small molecule.

In some instances, the expression cassette may also encode artificial miRNA. In some embodiments, the artificial miRNA may be configured to target endogenous, affected gene, as well as nucleotide sequences to improve RNA stability. In some embodiments, the nucleotide sequences to improve RNA stability may comprise WPRE. In some embodiments, the expression cassette (e.g., the nucleotide sequences) may be configured to detarget expression from liver and/or DRG.

Methods for treating Charcot-Marie-Tooth Diseases

Charcot-Marie-Tooth Diseases (CMT) is a neurodegenerative disorder that causes abnormalities in both sensory and motor nerves related to feet, legs, hands, and arms. CMT is a heterogenous group of peripheral neuropathies (e.g., affects nerves outside of the brain and spinal cord) affecting dorsal root ganglia and peripheral nerves. CMT is related to dysfunction of one of several genes, including, but not limited to: PMP22, GJB1, MFN2, TRPV4, NEFH, NEFL, DNM2, MPZ.

Certain aspects of the present disclosure relate to methods of treating Charcot-Marie-Tooth Diseases (CMT) in an individual in need thereof. In some embodiments, the method of treating CMT may comprise administering an effective amount of a viral particle of the disclosure. In some embodiments, the expression cassette of the viral particle may comprise a transgene. In some embodiments, the transgene may comprise PMP22, GJB1, MFN2, TRPV4, NEFH, NEFL, DNM2, or MPZ.

In some embodiments, the method for treating CMT may comprise delivering a viral particle comprising expression cassette encoding artificial miRNA. In some embodiments the artificial miRNA may be configured to target PMP22 or TRPV4. In some embodiments, the expression cassette may be configured to ‘encode genes comprising GJB1 or MFN1’. In some embodiments, the expression cassette may be configured to encode both an artificial miRNA targeting MFN2, NEFH, NEFL, DNM2, or MPZ along with targeted genes comprising GJB1 or MFN1.

In some embodiments, the expression cassette is under control of a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, the promoter may comprise a Schwann cell-specific promoter. In some embodiments, the Schwann cell-specific promoter may comprise MBP, MPZ, or PMP22. In some embodiments, promoter activity may be regulated by an exogeneous small molecule.

In some instances, transgene expression may be regulated by an exogenous small molecule.

In some instances, the expression cassette of the viral particle may comprise at least one additional nucleotide sequence. In some embodiments, at least one additional nucleotide sequence may be configured to improve RNA stability. In some embodiments, at least nucleotide sequence configured to improve RNA stability may comprise WPRE. In some embodiments, at least one additional nucleotide sequence may comprise a nucleotide sequence configured to detarget expression from the liver.

Methods for treating Fabry Disease

Fabry Disease (FD), otherwise known as alpha-galactosidase-A deficiency occurs when the enzyme alpha-galactosidase-A is unable to break down lipids. The buildup of lipid levels negatively affects function of the cerebral vasculature, heart, kidney, peripheral nervous system, and cerebral cortex. FD is caused by variants of the GLA gene that do not properly encode the Alpha-galactosidase-A enzyme.

Certain aspects of the present disclosure relate to methods of treating Fabry Disease (FD) in an individual in need thereof. In some embodiments, the method of treating FD may comprise administering an effective amount of a viral particle of the disclosure. In some embodiments, the expression cassette may comprise a transgene. In some embodiments, the transgene may comprise human GLA.

In some embodiments, the expression cassette of the viral particle is under control of a ubiquitous promoter or a neuron-specific promoter. In some embodiments, the ubiquitous promotor may comprise CBA, CAG, CAGG, CMV, H1, U6, or 7SK. In some embodiments, the Schwann cell-specific promoter may comprise MBP, MPZ, or PMP22. In some embodiments, promoter activity may be regulated by an exogeneous small molecule.

In some embodiments, the transgene expression may be regulated by an exogeneous small molecule.

In some instances, the human GLA gene may be modified to include cell penetrating and/or signal peptides to enhance secretion and uptake.

In some instances, the expression cassette of the viral particle may comprise at least one additional nucleotide sequence. In some embodiments, at least one additional nucleotide sequence may be configured to improve RNA stability. In some embodiments, at least nucleotide sequence configured to improve RNA stability may comprise WPRE. In some embodiments, the at least one additional nucleotide sequence may be configured to detarget expression from liver and/or DRG.

Methods for treating Angelman Syndrome

Angelman Syndrome (AS) is a neurodevelopmental disorder resulting in delayed development, problems with speech and balance, intellectual disability, and, sometimes, seizures. AS is caused by dysfunction of the UBE3A gene affecting the cerebral cortex. UBE3A encodes an E3 ubiquitin-protein ligase, which is part of the ubiquitin protein degradation system and is maternally expressed in the brain and spinal cord (central nervous system). AS is caused by materially inherited deletions of UBE3A.

Certain aspects of the present disclosure relate to methods of treating Angelman Syndrome (AS) in an individual in need thereof. In some embodiments, the method of treating AS may comprise administering an effective amount of a viral particle of the disclosure. In some embodiments, the expression cassette may comprise a transgene. In some embodiments, the transgene may comprise UBE3A. In some embodiments, the viral particle comprises an expression cassette encoding human UBE3A may also comprise an artificial miRNA. In some embodiments, the artificial miRNA may be configured to target UBE3A-ATS.

In some embodiments, the expression cassette is under control of a promoter. In some embodiments, the expression cassette is under control of a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, promoter activity may be regulated by an exogeneous small molecule.

In some embodiments, the method for treating AS may comprise expressing a transgene regulated by an exogeneous small molecule.

In some instances, the expression cassette may comprise at least one additional nucleotide sequence. In some embodiments, the at least one additional nucleotide sequence may be configured to improve RNA stability. In some embodiments, the at least nucleotide sequence configured to improve RNA stability may comprise WPRE. In some embodiments, the at least one additional nucleotide sequence may be configured to detarget expression from liver and/or DRG.

### Methods for Treating Fragile X Syndrome

Fragile X Syndrome (FXS) is a neurodevelopmental disorder caused by dysfunction of the FMR1 gene. FMR1 usually makes a protein called FMRP that is needed for brain development. People with FXS have the dysfunctional FMR1 gene, which results in developmental delays, learning disabilities and social and behavioral problems.

In some embodiments, the disclosure provides methods of treating Fragile X Syndrome (FXS) by administering an effective amount of a viral particle of the disclosure for expressing/encoding a disorder-related polypeptide of the present disclosure. In some embodiments, the disorder-related polypeptide may comprise human FMRP.

In some embodiments, the expression cassette is under control of a promoter. In some embodiments, the expression cassette is under control of a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, promoter activity may be regulated by an exogeneous small molecule.

In some embodiments, the expression cassette may comprise a transgene. In some embodiments, the transgene may comprise FMR1. In some embodiments, the transgene expression may be regulated by an exogenous small molecule.

In some embodiments, the expression cassette for expressing a disorder-related polypeptide may be administered through various routes as provided herein.

In some instances, the expression cassette may comprise at least one additional nucleotide sequence. In some embodiments, the at least one additional nucleotide sequence may be configured to improve RNA stability. In some embodiments, the at least nucleotide sequence configured to improve RNA stability may comprise WPRE. In some embodiments, the at least one additional nucleotide sequence may be configured to detarget expression from liver and/or DRG.

Methods for treating Rett Syndrome

Rett Syndrome (RS) is a progressive neurodevelopmental disorder and a common cause of cognitive disability. The most common causes of RS are mutated variants of the MECP2 gene which affect the cerebral cortex. These gene mutations alter the structure of the MeCP2 protein or reduce the amount produced. The reduction of functional MeCP2 can impair the regulation of gene expression in brain cells and can disrupt alternative splicing of proteins that are important for communications between neurons.

In some embodiments, the disclosure provides methods of treating Rett Syndrome (RS) by administering an effective amount of a viral particle of the disclosure for expressing/encoding a disorder-related polypeptide of the present disclosure. In some embodiments, the disorder-related polypeptide is a wild type disorder-related polypeptide. In some embodiments, the disorder-related polypeptide may comprise methyl CpG binding protein 2 (MECP2).

In some embodiments, the expression cassette is under control of a promoter. In some embodiments, the expression cassette is under control of a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, promoter activity may be regulated by an exogeneous small molecule.

In some embodiments, the expression cassette may comprise a transgene. In some embodiments, the transgene may comprise the transgene MECP2. In some embodiments, the transgene expression may be regulated by an exogenous small molecule.

In some embodiments, the expression cassette for expressing a disorder-related polypeptide may be administered through various routes as provided herein.

In some instances, the expression cassette may comprise at least one additional nucleotide sequence. In some embodiments, at least one additional nucleotide sequence may be configured to improve RNA stability. In some embodiments, at least nucleotide sequence configured to improve RNA stability may comprise WPRE. In some embodiments, at least one additional nucleotide sequence may be configured to detarget expression from liver and/or DRG.

Methods for treating Tuberous Sclerosis Complex

Tuberous Sclerosis Complex (TSC) is a rare genetic disorder caused by dysfunction of the TSC1 or TSC2 genes that results in spontaneous benign tumor growth in multiple organs throughout the body. TSC1 is a tumor suppressor gene that encodes the growth inhibitory protein hamartin. TSC2 is a tumor suppressor gene that encodes the growth inhibitory protein tuberin. Tuberin interacts with hamartin to form the TSC protein complex which functions in the control of cell growth. Dysfunction of TSC1 or TSC2 results in growth of tumors in tissues including but not limited to the brain, skin, heart, lungs, kidneys, and liver. Tumor growth in the cerebral cortex and ventricular space can cause seizure and hydrocephaly leading to neurodevelopmental delay, neurological disorders and in rare cases death.

In some embodiments, the disclosure provides methods of treating Tuberous Sclerosis Complex (TSC) by administering an effective amount of a viral particle of the disclosure for expressing/encoding a disorder-related polypeptide of the present disclosure. In some embodiments, the disorder-related polypeptide is a wild type disorder-related polypeptide. In some embodiments, the disorder-related polypeptide may comprise hamartin, tuberin, or a combination of both.

In some embodiments, the expression cassette is under control of a promoter. In some embodiments, the expression cassette is under control of a ubiquitous promoter or a neuron-specific promoter. In some embodiments, the ubiquitous promotor may comprise CBA, CAG, CAGG, CMV, H1, U6, or 7SK. In some embodiments, promoter activity may be regulated by an exogeneous small molecule.

In some embodiments, the expression cassette may comprise at least one transgene. In some embodiments, the at least one transgene may comprise the transgene TSC1, TSC2, or a combination of both. In some embodiments, the transgenic payload may comprise all or part of human TSC1 and/or TSC2 human cDNA. In some embodiments, the transgenic payload may comprise all or part of human TSC1 and/or TSC2 human cDNA that may include additional flexible linker sequence. In some embodiments, the transgene expression may be regulated by an exogenous small molecule.

In some instances, the expression cassette may be modified to include cell penetrating and/or signal peptides to enable secretion and uptake.

In some instances, the genetic payload may comprise at least one additional nucleotide sequence. In some embodiments, the at least one additional nucleotide sequence may comprise a nucleotide sequence to facilitate nuclear export. In some embodiments, the at least one additional nucleotide sequence may comprise a nucleotide sequence to improve RNA stability. In some embodiments, the nucleotide sequence to improve RNA stability may comprise a poly A tail. In some embodiments, the nucleotide sequence to improve RNA stability may comprise WPRE. In some embodiments, at least one additional nucleotide sequence may be configured to detarget expression from liver and/or DRG.

In some instances, the expression cassette may comprise at least one tagging nucleotide sequence. In some embodiments, at least one tagging nucleotide sequence may comprise FLAG, His, Myc or any combination thereof.

In some embodiments, the viral particle for expressing a disorder-related polypeptide may be administered through various routes as provided herein. In some embodiments, AAV administration may be either intra-CSF (intrathecal, intra-cisterna magna, or intraventricular) or intravenous.

Methods for treating Neurofibromatosis type II

Neurofibromatosis type II (NF2) is a disorder characterized by nervous system and skin tumors and ocular abnormalities. Disruption in the function of the protein encoded by the NF2 gene has been implicated in tumorigenesis and metastasis. Dysfunction in the NF2 gene is caused by spontaneous genetic mutation.

In some embodiments, the disclosure provides methods of treating Neurofibromatosis type II (NF2) by administering an effective amount of a viral particle of the disclosure for expressing a disorder-related polypeptide. In some embodiments, the disorder-related polypeptide may comprise a polypeptide expressed by the gene NF2.

In some embodiments, the expression cassette of the viral particle is under control of a ubiquitous promoter, a neuron-specific promoter, or a Shwann cell-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, the Schwann cell-specific promoter may comprise MBP, MPZ, or PMP22. In some embodiments, promoter activity may be regulated by an exogeneous small molecule.

In some embodiments, the expression cassette may comprise at least one transgene. In some instances, transgene expression may be regulated by an exogenous small molecule. In some embodiments, the transgene may comprise human NF2. In some embodiments, the transgenic payload may comprise all or part of the human NF2 gene, an additional flexible linker sequence, or a combination of both.

In some instances, the expression cassette (e.g., AAV genome, genetic payload, etc.) may be modified to include cell penetrating and/or signal peptides to enable secretion and uptake.

In some instances, the expression cassette may comprise at least one additional nucleotide sequence. In some embodiments, at least one additional nucleotide sequence may comprise a nucleotide sequence configured to detarget expression from liver and/or DRG.

In some instances, the expression cassette may comprise at least one tagging nucleotide sequence. In some embodiments, at least one tagging nucleotide sequence may comprise FLAG, His, Myc or any combination thereof.

In some embodiments, the viral particle for expressing a disorder-related polypeptide may be administered through various routes as provided herein. In some embodiments, AAV administration may be either intra-CSF (intrathecal, intra-cisterna magna, or intraventricular) or intravenous.

### Methods for Treating Pompe

Pompe is a neuromuscular disease with a caused by autosomal recessive mutations in the acid alpha-glucosidase (GAA) gene. The GAA gene encodes lysosomal alpha-glucosidase, which is essential for the degradation of glycogen to glucose in lysosomes. The hallmark pathology of Pompe is glycogen storage in the lysosomes of heart, skeletal muscles and central nervous system, which lead to hypotonia, cardiomyopathy, respiratory deficiency, and neurological symptoms or even pre-mature death of patients with early onset.

In some embodiments, the disclosure provides methods of treating Pompe by administering an effective amount of a viral particle of the disclosure for expressing/encoding a disorder-related polypeptide of the present disclosure. In some embodiments, the disorder-related polypeptide comprises the lysosomal enzyme acid alpha-glucosidase (GAA). In some embodiments, the expression cassette is configured for encoding the human GAA polypeptide.

In some embodiments, the expression cassette may be under control of a promoter. In some embodiments, the promoter may comprise a ubiquitous promoter, or a muscle-specific promoter. In some embodiments, the ubiquitous promotor may comprise CBA, CAG, CAGG, CMV, H1, U6, or 7SK. In some embodiments, the muscle-specific promoter may comprise Spc5-12, Desmin, or αMyHC. In some embodiments, promoter activity may be regulated by an exogeneous small molecule.

In some embodiments, the expression cassette may comprise at least one transgene. In some embodiments, at least one transgene may comprise the human GAA gene. In some instances, transgene expression may be regulated by an exogenous small molecule.

In some instances, the human GAA gene may be modified to comprise cell penetrating and/or signal peptides. In some instances, the human GAA gene may comprise cell penetrating and/or signal peptides to enable secretion and uptake.

In some instances, the expression cassette of the viral particle may comprise at least one additional nucleotide sequence. In some embodiments, at least one additional nucleotide sequence may be configured to improve RNA stability. In some embodiments, at least one additional nucleotide sequence configured to improve RNA stability may comprise WPRE. In some embodiments, the at least one additional nucleotide sequence configured to detarget expression from liver and/or DRG.

In some embodiments, the expression cassette for expressing a disorder-related polypeptide may be administered through various routes as provided herein. In some embodiments, the method for treating Pompe may comprise delivering the viral particle via IV and/or intra-CSF routes to target both the skeletal muscle and CNS components of the disease.

Methods for treating Globoid cell Leukodystrophy

Globoid cell leukodystrophy (GLD; Krabbe disease) an autosomal recessive neurodegenerative disorder caused by a defect in the GALC gene. The GALC gene encodes for the lysosomal enzyme, galactosylceramidase. Without the enzyme, buildup of cytotoxic galactosylsphingosine, a GALC substrate, drives death of oligodendrocytes and Schwann cells, leading to widespread myelin loss.

In some embodiments, the disclosure provides methods of treating Globoid cell leukodystrophy (GLD) by administering an effective amount of a viral particle of the disclosure for expressing/encoding a disorder-related polypeptide of the present disclosure. In some embodiments, the disorder-related polypeptide comprises the lysosomal enzyme, galactosylceramidase. In some embodiments, the method for treating GLD is configured to prevent buildup of cytotoxic galactosylsphingosine, a GALC substrate, which drives death of oligodendrocytes and Schwann cells, leading to widespread myelin loss.

In some embodiments, the expression cassette of the viral particle is under control of a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, TUBB3. In some embodiments, the promoter may comprise a Schwann cell-specific promoter. In some embodiments, the Schwann cell-specific promoter may comprise MBP, MPZ, or PMP22. In some embodiments, promoter activity may be regulated by an exogeneous small molecule.

In some embodiments, the expression cassette may comprise at least one transgene. In some embodiments, at least one transgene may comprise the human GALC gene. In some instances, transgene expression may be regulated by an exogenous small molecule.

In some instances, the human GALC gene may be modified to comprise cell penetrating and/or signal peptides to enable secretion and uptake.

In some instances, the expression cassette may comprise at least one additional nucleotide sequence. In some embodiments, at least one additional nucleotide sequence may be configured to improve RNA stability. In some embodiments, at least one additional nucleotide sequence may comprise WPRE. In some embodiments, at least one additional nucleotide sequence may be configured to detarget expression from liver and/or DRG.

In some embodiments, the viral particle may be administered through various routes as provided herein. In some embodiments, the method for treating GLD may comprise delivering an expression cassette (e.g., AAV genome) via IV and/or intra-CSF routes to target both the PNS and CNS components of the disease.

### Methods for Treating Multiple Sclerosis

Globoid cell leukodystrophy (GLD; Krabbe disease) an autosomal recessive neurodegenerative disorder caused by a defect in the GALC gene. The GALC gene encodes for the lysosomal enzyme, galactosylceramidase. Without the enzyme, buildup of cytotoxic galactosylsphingosine, a GALC substrate, drives death of oligodendrocytes and Schwann cells, leading to widespread myelin loss.

In some embodiments, the disclosure provides methods of treating Multiple Sclerosis (MS) by administering an effective amount of a viral particle of the disclosure for expressing/encoding a disorder-related polypeptide. In some embodiments, the disorder-related polypeptide comprises a vectorized antibody against CD40L.

In some embodiments, the expression cassette of the viral particle is under control of a promoter. In some embodiments, the promoter comprises a ubiquitous promoter or a neuron-specific promoter. In some embodiments, the ubiquitous promotor may comprise CBA, CAG, CAGG, CMV, H1, U6, or 7SK. In some embodiments, the neuron-specific promoter may comprise SYN1, NSE, CAMKII, or TUBB3. In some embodiments, promoter activity may be regulated by an exogeneous small molecule.

In some embodiments, the expression cassette may comprise at least one transgene. In some embodiments, the at least one transgene may encode for a vectorized antibody against CD40L. In some instances, transgene expression may be regulated by an exogenous small molecule.

In some instances, the vectorized antibody may be modified to comprise cell penetrating and/or signal peptides to enable secretion and uptake.

In some instances, the expression cassette may comprise at least one additional nucleotide sequence. In some instances, the expression cassette (e.g., AAV genome) may comprise at least one additional nucleotide sequence configured to improve RNA stability. In some embodiments, at least one additional nucleotide sequence configured to improve RNA stability may comprise WPRE. In some embodiments, at least one additional nucleotide sequence configured to de-target expression from liver and/or DRG.

In some embodiments, the viral particle for expressing a disorder-related polypeptide may be administered through various routes as provided herein.

### Methods for Treating Gaucher Disease

Gaucher Disease (GD) is an autosomal recessive lysosomal storage disorder caused by mutations in GBA1, the gene encoding glucocerebrosidase (GCase). Reduction or loss of GCase activity leads to the accumulation of toxic lipid substrates, disrupting cellular homeostasis. GD is further GD Type 1 (GD1) patients typically present with splenomegaly, hepatomegaly, and anemia or thrombocytopenia, while GD Type 3 (GD3) patients present with debilitating neurological symptoms and associated systemic manifestations of GD1. GD Type 2 (GD2) is the most severe form of the disease affecting infants before 6 months of age and in most cases causing early death by 2-3 years. Like GD3, GD2 also has CNS symptoms at a much higher severity. Current treatment landscape for GD includes enzyme replacement therapy for GD1 patients that alleviate symptoms but since ERTs fail to enter the brain, they do not help GD2 and GD3 patients that have CNS symptoms. Furthermore, GD1 patients have a lifelong burden of having to take bi-weekly infusion of the ERT.

In some embodiments, the disclosure provides methods of treating Gaucher Disease (GD) by administering an effective amount of a viral particle of the disclosure for expressing/encoding glucocerebrosidase (GCase). In some embodiments, the GCase (e.g., GCase peptide) is a wild type peptide. In some embodiments, the method may comprise restoring the loss of GCase activity. In some embodiments, given the disease symptoms, intra-CSF dosing for GD2 and GD3; IV dosing for GD1 of the same AAV gene therapy product would be ideal.

### Methods for Treating GBA-PD

About 5-10% of Parkinson's disease (PD) patients which is 0.5-1 million are carriers of GBA1 mutation in one of their alleles. GBA1 is an extremely well-credentialed target in GBA-PD. Just like with sporadic PD, there are no disease-modifying therapies yet. Current treatment options include Levodopa and/or dopamine agonists for symptom management but most patients notice the effects of these drugs wearing off over time.

In some embodiments, the disclosure provides methods of treating GBA-PD by administering an effective amount of an expression cassette (e.g., an expression cassette delivered in a rAAV particle) for expressing/encoding GBA1 polypeptide. In some embodiments, the method for treating GBA-PD may comprise a one-time administration of AAV-GBA1 delivered via intra-CSF administration to specifically target the CNS.

### Methods for Treating Huntington's Disease (HD)

HD is caused by a combination of hereditary and somatic expansion of trinucleotide repeats in the gene HITT that drives HD pathology in cerebral cortex, caudate, and putamen. Somatic instability of existing expansions is linked to the activity of DNA replication and repair components encoded by the genes including but not limited to MSH2, MSH3, PMS1, PMS2, FAN1, RRM2B, LIG1, TCERG1, and MLH3.

In some embodiments, the viral particles of the disclosure can be used for the treatment of HD. In some embodiments, treatment of HD involves delivery of an AAV genome encoding artificial microRNA, antisense oligonucleotide, linearized antibody, or nanobody targeting the HTT gene and/or MSH2, MSH3, PMS1, PMS2, FAN1, RRM2B, and MLH3, either alone or in combination, under control of ubiquitous (CBA, CAG, CAGG, CMV, H1, U6, 7SK, or others to be determined) or neuron-specific (SYN1, NSE, CAMKII, TUBB3, or others to be determined) promoters. In some instances, promoter activity and/or transgene expression may be regulated by an exogenous small molecule. In other instances, treatment may encode biological machinery, including DNA binding protein, effector protein, and/or nucleotide guide molecule, required for editing to remove repeat expansions from the HTT gene.

### Kits or Articles of Manufacture

The expression cassettes (e.g., an expression cassette for expressing a disorder-related polypeptide, such as a wild type human disorder-related polypeptide), rAAV vectors, particles, and/or pharmaceutical compositions as described herein may be contained within a kit or article of manufacture, e.g., designed for use in one of the methods of the disclosure as described herein.

Generally, the system comprises a cannula, one or more syringes (e.g., 1, 2, 3, 4 or more), and one or more fluids (e.g., 1, 2, 3, 4 or more) suitable for use in the methods of the disclosure.

The syringe may be any suitable syringe, provided it is capable of being connected to the cannula for delivery of a fluid. In some embodiments, the system has one syringe. In some embodiments, the system has two syringes. In some embodiments, the system has three syringes. In some embodiments, the system has four or more syringes. The fluids suitable for use in the methods of the disclosure include those described herein, for example, one or more fluids each comprising an effective amount of one or more vectors as described herein, and one or more fluids comprising one or more therapeutic agents.

In some embodiments, the kit comprises a single fluid (e.g., a pharmaceutically acceptable fluid comprising an effective amount of the vector). In some embodiments, the kit comprises 2 fluids. In some embodiments, the kit comprises 3 fluids. In some embodiments, the kit comprises 4 or more fluids. A fluid may include a diluent, buffer, excipient, or any other liquid described herein or known in the art suitable for delivering, diluting, stabilizing, buffering, or otherwise transporting an expression cassette for expressing a disorder-related polypeptide or rAAV vector composition of the present disclosure. In some embodiments, the kit comprises one or more buffers, e.g., an aqueous pH buffered solution. Examples of buffers may include without limitation phosphate, citrate, Tris, HEPES, and other organic acid buffers.

In some embodiments, the kit comprises a container. Suitable containers may include, e.g., vials, bags, syringes, and bottles. The container may be made of one or more of a material such as glass, metal, or plastic. In some embodiments, the container is used to hold a rAAV composition of the present disclosure. In some embodiments, the container may also hold a fluid and/or other therapeutic agent.

In some embodiments, the kit comprises an additional therapeutic agent with a rAAV composition of the present disclosure. In some embodiments, the rAAV composition and the additional therapeutic agent may be mixed. In some embodiments, the rAAV composition and the additional therapeutic agent may be kept separate. In some embodiments, the rAAV composition and the additional therapeutic agent may be in the same container. In some embodiments, the rAAV composition and the additional therapeutic agent may be in different containers. In some embodiments, the rAAV composition and the additional therapeutic agent may be administered simultaneously. In some embodiments, the rAAV composition and the additional therapeutic agent may be administered on the same day. In some embodiments, the rAAV composition may be administered within one day, two days, three days, four days, five days, six days, seven days, two weeks, three weeks, four weeks, two months, three months, four months, five months, or six months of administration of the additional therapeutic agent.

In some embodiments, the kit comprises a therapeutic agent to transiently suppress the immune system prior to AAV administration. In some embodiments, patients are transiently immune suppressed shortly before and after injection of the virus to inhibit the T cell response to the AAV particles (e.g., see Ferreira et al., Hum. Gene Ther. 25:180-188, 2014). In some embodiments, the kit further provides cyclosporine, mycophenolate mofetil, and/or methylprednisolone.

The rAAV particles and/or compositions of the disclosure may further be packaged into kits including instructions for use. In some embodiments, the kits further comprise a device for delivery (e.g., any type of parenteral administration described herein) of compositions of rAAV particles. In some embodiments, the instructions for use include instructions according to one of the methods described herein. In some embodiments, the instructions are printed on a label provided with (e.g., affixed to) a container. In some embodiments, the instructions for use include instructions for administering to an individual (e.g., a human) an effective amount of rAAV particles, e.g., for treating a neurodegenerative disease in an individual.

### EXAMPLES

The invention will be more fully understood by reference to the following examples. They should not, however be construed as limiting the scope of the invention. It is understood that the examples and embodiments described herein are for illustrative purposes only and that various modification or changes in light thereof will be suggested to persons skilled in the art and are to be included within the spirit and purview of this application and scope of the appended embodiments.

### General Methods

### Tissue Homogenization

Tissues were homogenized with added cold 10 mM Tris 1 mM EDTA buffer in 1.4 mm ceramic bead homogenization tubes in refrigerated Omni bead ruptor-20 second cycle 4.7 oscillation/sec. Following homogenization, aliquots made for subsequent assay.

### BCA Assay

Total protein concentration determined by BCA (bicinchoninic acid) assay (Thermo Scientific 23227) using 10 μl supernatant diluted in water. Colorimetric detection of the cuprous cation (Cu¹⁺) by bicinchoninic acid (BCA) by absorbance at 562 nm. Molecular Devices SpectraMax 340PC-384 with SoftMax Pro version 5.4.4 software used to read 96 well microtiter plate.

### Nitroblue Myelin Stain

Transfer sections to 12-well plate and perform all steps in free-floating sections. Wash section 2×1 min in TBA on shaker. Incubate in Nitroblue solution (Sigma cat #N5374) for 1 hour and rinse 2×5 min in TBA on shaker. Incubate sections in DAB stain (Sigma Kit cat #D4293) for 1 hour and rinse 2×1 in RO water. Rinse sections in PBS for 1 min, mount in PBS and air dry slides. Counterstain with Neutral Red solution for 2 min, wash 2×2 in RO water. Rinse slides for 1 min in 95% reagent alcohol, 2×2 min in 100% reagent alcohol, 2×2 min in Xylene, coverslip using Acrytol mounting medium.

### GFP ELISA

Measurement of GFP protein was completed using the GFP SimpleStep ELISA® Kit from Abcam (ab171581). It is imperative to read the protocol insert provided with each kit lot as some reagent concentrations change with different lots, particularly the standard.

Prior to beginning, all kit components were equilibrated to room temperature (18-25C), at least 30 minutes. An aliquot of protein-processed tissue homogenate was thawed on ice. Reagents and working standards were prepared fresh, as per kit instructions. Samples were diluted in complete cell extraction buffer as follows: Grey Matter 1:5, Spinal Cord 1:5, Heart: 1:5, liver 1:20. Diluted sample and standard were added to the appropriate wells, followed by antibody cocktail to each well. The plate was sealed, incubated at room temperature for 1 hour, shaking at 400 RPM. Wells were washed 3 times with wash buffer, and complete removal of liquid was ensured. TMB solution was added to each well and incubated in the dark for 10 minutes at room temperature, shaking at 400 RPM. Quickly after TMB incubation, stop solution was added to each well and mixed on a plate shaker for 1 minute. The plate was read at 450 nm on a SpectraMax plate reader (Molecular Devices) using Softmax software.

### Flag IHC

The NHP brain FFPE slides were treated in EDTA solution (pH.9.0) for 20 minutes at 90° C. for antigen retrieval, followed by blocking with 3% hydrogen peroxide for 5 minutes. The slides were then incubated with Flag DDK antibody (Abcam ab205606) at 1:200 dilution in antibody diluent (Cell Signaling #8112) for one hour at room temperature followed by anti-rabbit HRP (Abcam ab6721) secondary antibody incubation. The color development for Flag DDK signal was achieved by incubating slides in DAB solution for 3 minutes at room temperature, and the slides were subsequently counter stained with hematoxylin for nuclei staining.

### GFP IHC

The NHP brain FFPE slides were treated in EDTA solution (pH.9.0) for 20 minutes at 90° C. for antigen retrieval and subsequently blocked with 3% hydrogen peroxide for 10 minutes, followed with 5% horse serum for 45 minutes at room temperature. The slides were then incubated with GFP antibody (ThermoFisher A-11122) at 1:500 dilution in antibody diluent (Cell Signaling #8112) for one hour at room temperature followed with anti-Rabbit HRP (Abcam ab6721) secondary antibody incubation. The color development for GFP signal was achieved by incubating slides in DAB solution for 3 minutes at room temperature, and the slides were counter stained with hematoxylin for nuclei staining.

### Single-Nuclei Sequencing

Nuclei Isolation: For nuclei isolation, dissected brains were transferred to microcentrifuge tubes, snap frozen in a slurry of dry ice and ethanol, and stored at −800° C. until the time of use. To isolate nuclei, frozen mouse brains were put into nuclei lysis buffer containing 0.1% Triton-100 (Sigma-Aldrich), 1 mM DTT (Sigma-Aldrich (, and 0.2U/ul RNase Inhibitor (Sigma-Aldrich) in 1 ml Dounce Homogenizer (Wheaton). Tissue was homogenized using 10 strokes of the loose Dounce pestle followed by 10 strokes of the tight pestle, and incubated on ice for 15 min. The resulting homogenate was passed through 30 μm cell strainer (Miltenyi Biotech) and centrifuged at 500×g for 5 min to pellet nuclei. Nuclei were resuspended in buffer containing 1×PBS (Thermo Fisher), 1% nuclease-free BSA (Sigma-Aldrich), 1 mM DTT (Sigma-Aldrich) and 0.2U/ul RNase inhibitor (Sigma-Aldrich). Mouse anti-NeuN conjugated to PE (EMD Millipore) was added to preparations at a dilution of 1:500 and samples were incubated for 30 min at 40° C. Samples were then centrifuged for 5 min at 500xg to pellet nuclei, and pellets were resuspended in 1×PBS, 1% BSA, 1 mM DTT, and 0.2U/ul RNase inhibitor. DAPI was added at a concentration of 0.1 ug/ml. Single nuclei sorting was carried out on Influx-83 (BD Biosciences) using 100 μm nozzle. Nuclei were gated on DAPI and NeuN signal (PE).

### Library Preparation and NovaSeq Sequencing

Libraries were prepared according to 10×Genomics protocol for Chromium Single Cell 3′ Gene Expression V3.1 kit. Briefly, immediately after sorting, the nuclei were mixed with 90% NeuN- and 10% NeuN+nuclei. The GEM generation step in 10×Genomics gene expression kit (10×Genomics) was carried out. After GEM generation, samples were kept at −20° C. for further step. All samples were processed together from cDNA amplification, library construction, to libraries sequencing. The quantification of cDNA and libraries were solid for all samples. The libraries were sequenced on illumine Novaseq 6000. Libraries were sequenced at a median depth of around 50K reads/nuclei. UMI count matrices generated by Cell Ranger V5.

Data Preprocessing: The generated count matrices together with nucleus barcodes and gene labels were loaded onto Partek Flow. For Quality Control (QC), nuclei were filtered following standard protocols based on examination of violin plots. The detailed cutoffs were 250<nFeature RNA <6000 and nCount RNA <30000. The mitochondria protein coding genes were <2%. After quality filtering, 147313 total nuclei remained for the further analysis.

### Vector Genome Assessment

HT gDNA isolation: The gDNA was isolated from 50 μl NHP tissue homogenate using QIAmp 96 DNA QIAcube HT kit (cat #51331) according to manufacturer's protocol “QIAamp® 96 DNA QIAcube® HT Handbook” The tissue homogenate was treated with proteinase K at 56C overnight first, then transferred to S block and place the samples into QIAcube HT instrument performing gDNA isolation with the QIAcube HT Prep Mange Softer ware. gDNA concentration then was measured with NANODROP 8000 (Thermo Fisher Scientific).

dPCR via QIAcuty: Vector genome was determined with dPCR via QIAcuty that manufactured by QIAGEN. 7 μg of gDNA isolated from NHP tissue and 5 μl of master reaction mix which contain 1x probe PCR Master Mix (cat #250103); 1xprimer-probe mix1 (BGH) and mix2 (housekeep gene); 0.25U of HindIII restriction enzyme was mixed in standard PCR plate and then transferred to nanoplate. Total reaction volume is 12 μl per well. Then place the nanoplate into QIAcuity instrument and perform dPCR use manufacture suggested cycling. DNA copy for each sample and each gene was analyzed automatically by the softer ware. Then vector genome per cell was calculated as: 2XBGH copies divide housekeep gene copies.

### Example 1. AAV.SAN006 compared to AAVrh10 and AAV-Myo

This study was designed to evaluate biodistribution in terms of transgene expression as a function of viral dosage for two different capsids, AAV.SAN006 (also referred to herwin as SAN006) and AAVrh10. Briefly, Cynomolgus monkeys (Male, Mauritian 2 yr old, 2-3 kg) seronegative for AAVrh10, AAV-Myo and AAV.SAN006 (AAVSAN006) were dosed intra-thecal at the cervical level 1-2 junction using a ported intrathecal catheter inserted at the lumbar region. Animals were dosed in the Trendelenburg position with two 2.5 mL infusions at 0.125 mL/min, approximately 6 hours apart. One dose containing AAV.SAN006-CBA-eGFP, AAV-rh10-CBA-eGFP and AAV-myo-CBA-eGFP was administrated at 2.75e13 VG/NHP (at 3.3e11 VG/brain gram). Two weeks post-dosing, animals were euthanized, and samples were assessed for GFP expression by ELISA.

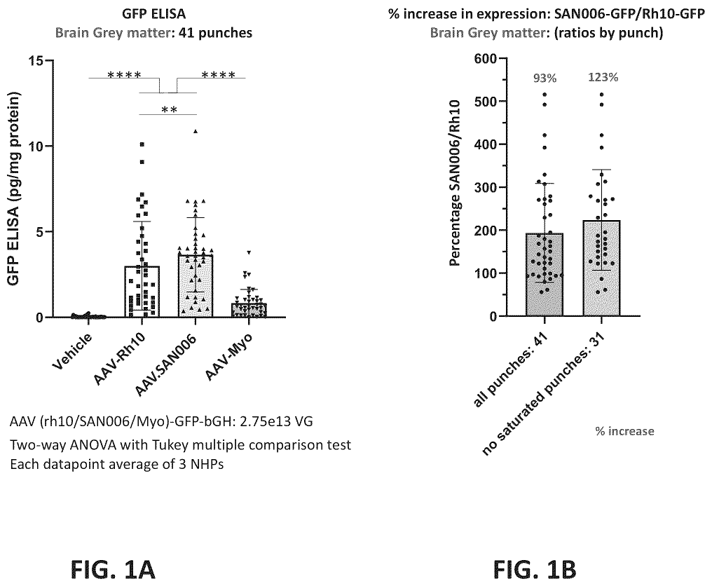

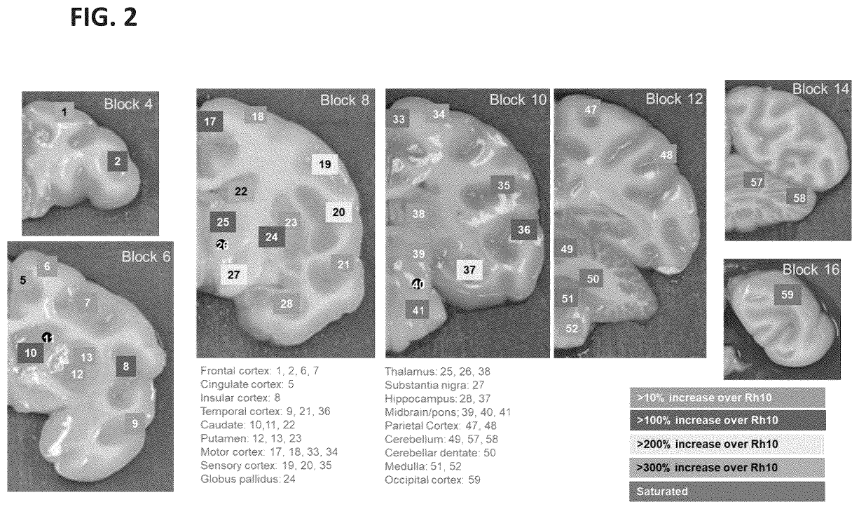

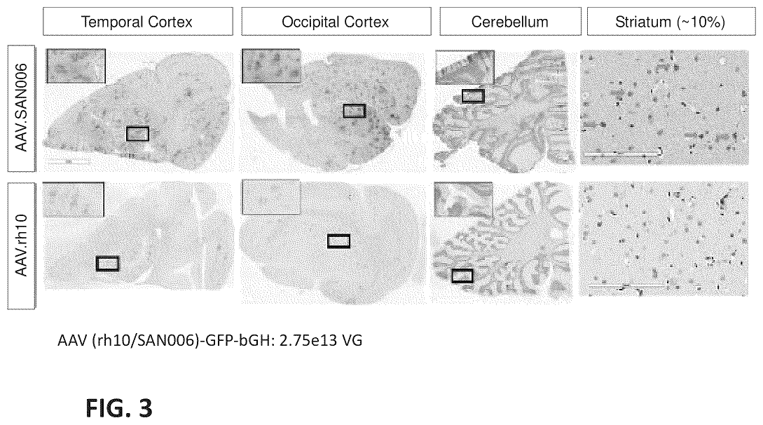

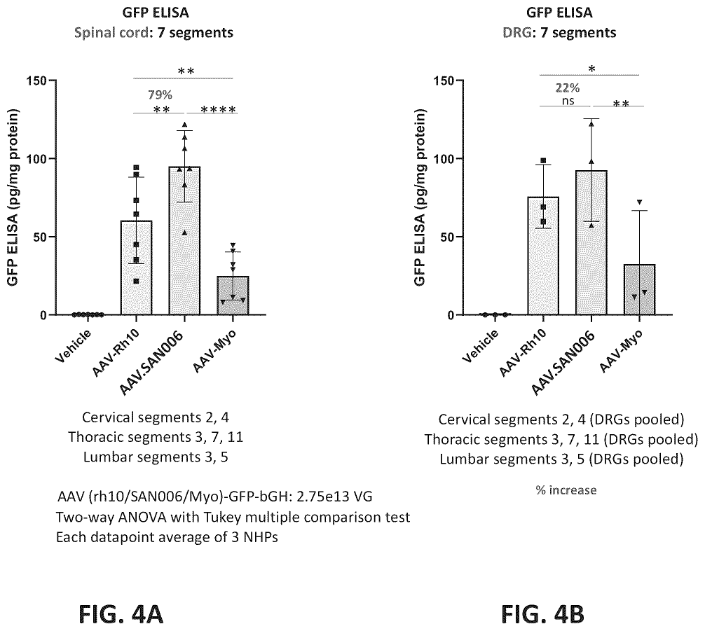

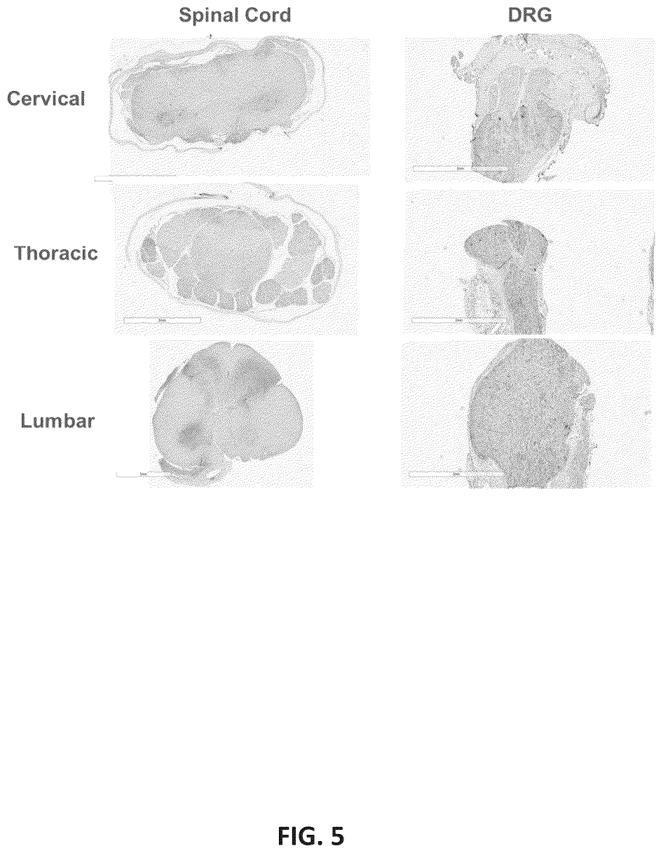

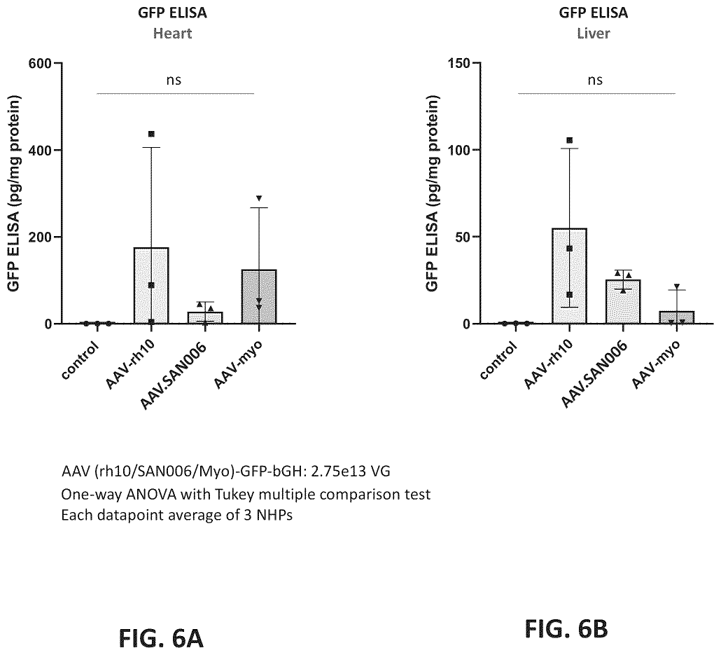

A total of 41 brain punches (grey matter) representing 16 different brain regions showed significantly higher GFP expression in NHPs dosed with AAV.SAN006 as compared to AAVrh10 dosed NHPs (FIG. 1A). Across all samples, GFP expression in brain of AAV.SAN006-treated NHPs was 93%-123% higher, as compared to AAVrh10-treated NHPs (FIG. 1B). This is demonstrated by the heatmap as depicted in FIG. 2, where in 38 out of 41 punches (averaged across three NHPs), AAV.SAN006 treated NHPs show at minimum 10% increase in GFP expression, compared to AAV-rh10 treated NHPs. Representative matched brain sections stained with antibodies at eGFP show robust expression in AAV.SAN006 treated NHPs, demonstrating superior biodistribution and expression as compared to AAV-rh10 as depicted in FIG. 3. Further, in the spinal cord and DRGs, GFP expression in AAV.SAN006-treated NHPs was 79% and 22% higher as compared to AAVrh 10-treated NHPs, as depicted in FIGS. 4A-5, respectively. FIGS. 6A-6B show AAV.SAN006-GFP expression in the heart and liver, respectively, were lower, compared to AAV-rh10-GFP treated NHPs.

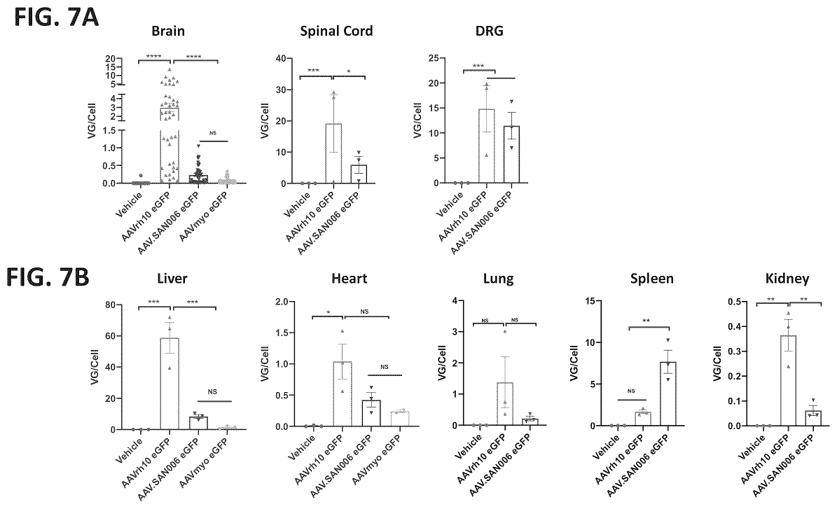

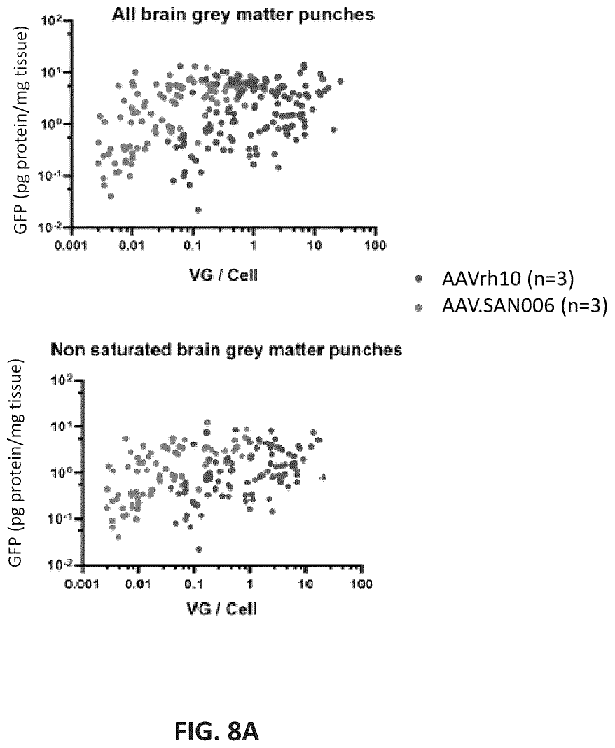

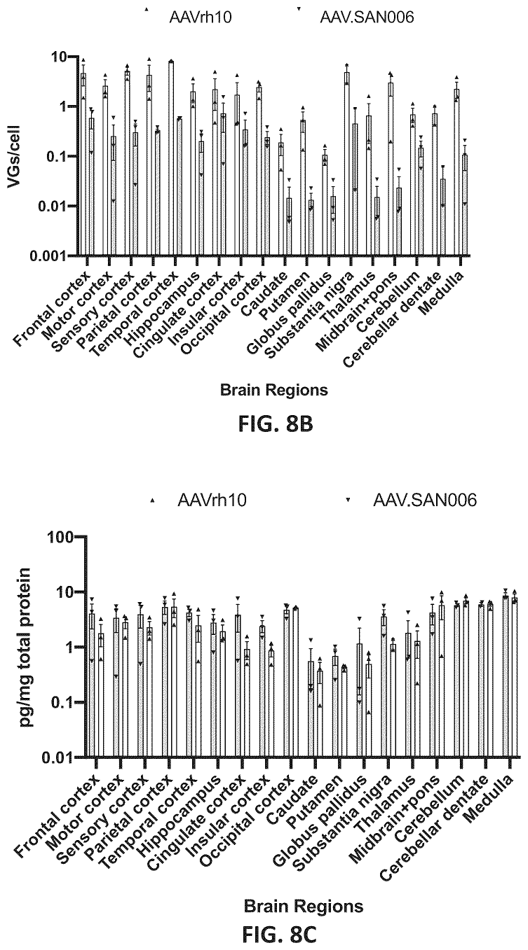

AAV.SAN006 vector genome exposure (e.g., vector genome load) in tissues was ten times lower in the brain and three times lower in the spinal cord, compared to AAVrh10 as depicted in FIGS. 7A-7B. This is noticeable in the ‘left-shift’ in the correlation between GFP expression (y-axis) and tissue vector genomes (x-axis), where in the correlation for AAV.SAN006 (red) is left shifted indicating higher protein expression and lower (˜10 fold) tissue dose of the vector as depicted in FIG. 8A. The high protein expression with low vector genome exposure in the brain is observed even when analyzing individual brain regions as depicted in FIGS. 8B-8C. Here, similar to higher protein expression is observed with SAN006, compared to AAV.rh10 as depicted in FIG. 8B, however at >10 fold decreased vector exposure in those respective brain regions as depicted in FIG. 8C. Furthermore, DRGs, liver, heart, lung, and kidney also show lower AAV.SAN006 vector genomes compared to AAVrh10. Spleen is the only organ tested that shows higher AAV.SAN006 vector genomes compared to AAVrh10 (FIGS. 7A-7B). These results indicate that AAV.SAN006 is superior to AAVrh10 and gives broader biodistribution and higher transgene expression at a lower dose.

### Example 2. AAV.SAN006 biodistribution across different intra-CSF administration routes

This study was designed to evaluate AAV.SAN006 biodistribution across different administration routes. Briefly, Cynomolgus monkeys (Male, Mauritian 2 yr old, 2-3 kg) seronegative for AAV.SAN006 were dosed with AAV.SAN006-NLuc-mCherry at 2.5e13 VG/NHP by one of three different intra-CSF routes of administration (ROAs): 1) Bilateral intracerebroventricular injection (ICV), 2) Intrathecal catheter threaded from lumbar level 5 to the cervical level/cisterna magna junction (IT-CM), and 3) direct injection to the cisterna magna (ICM). At four weeks post-dosing, animals were euthanized, and tissues were flash-frozen from brain, spinal cord, dorsal root ganglia (DRG), and peripheral tissues. Tissue punches corresponding to 20 brain regions and 4 spinal levels were further homogenized in 1x Tris EDTA buffer for downstream DNA isolation and nanoluciferease (nLuc) activity assays. DNA samples were assayed by bGH dPCR to quantify vector genomes per cell. Additional tissues from brain, spinal cord, and DRG were fixed in 10% neutral buffered formalin and submitted for histopathological analysis. Cerebrospinal fluid (CSF) samples were collected both prior to dosing (“prestudy”) and at the four-week necropsy timepoint and were assayed by Quanterix Simoa to quantify neurofilament light chain (NfL), a measure of neuronal injury.

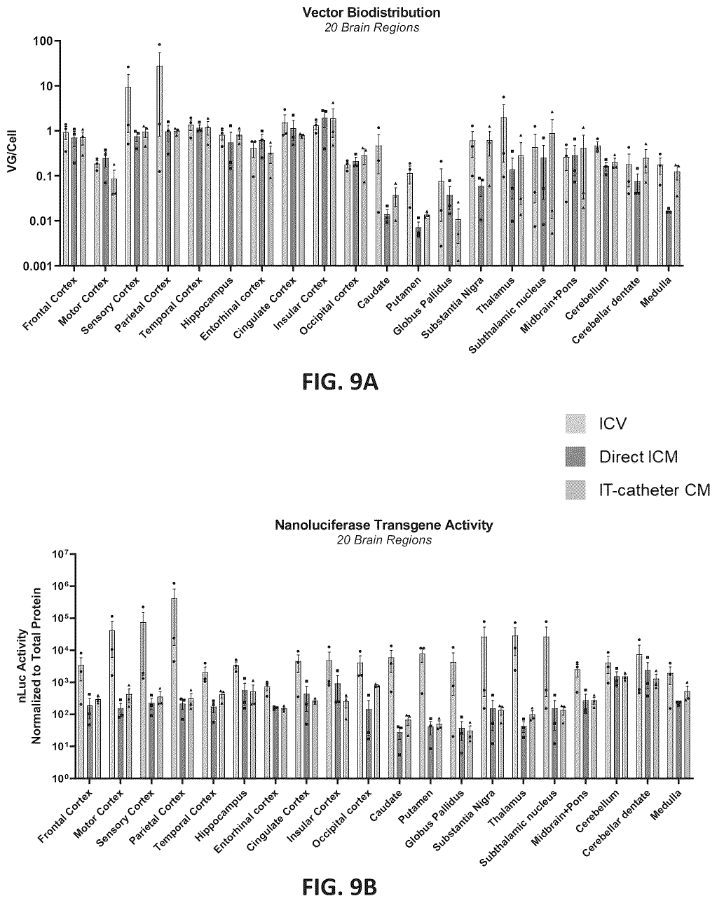

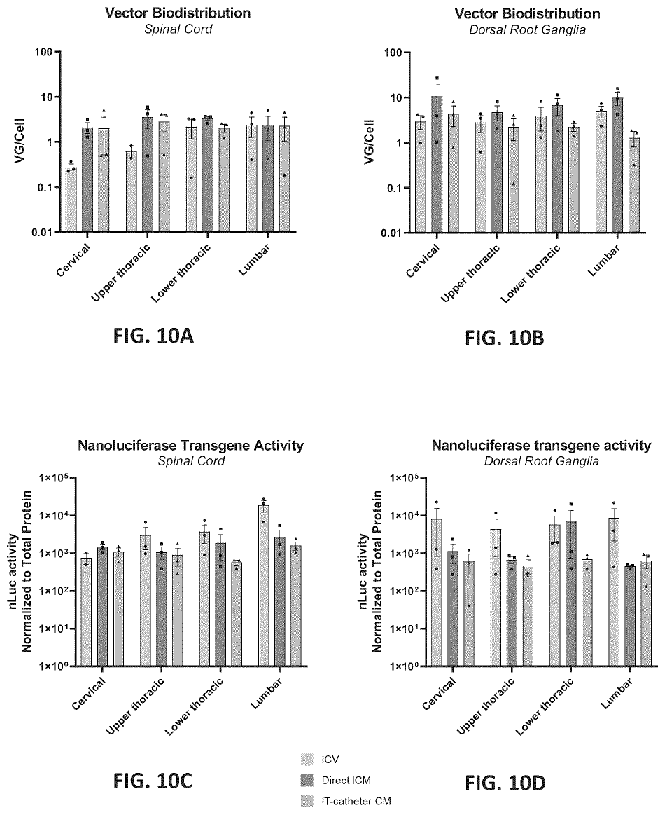

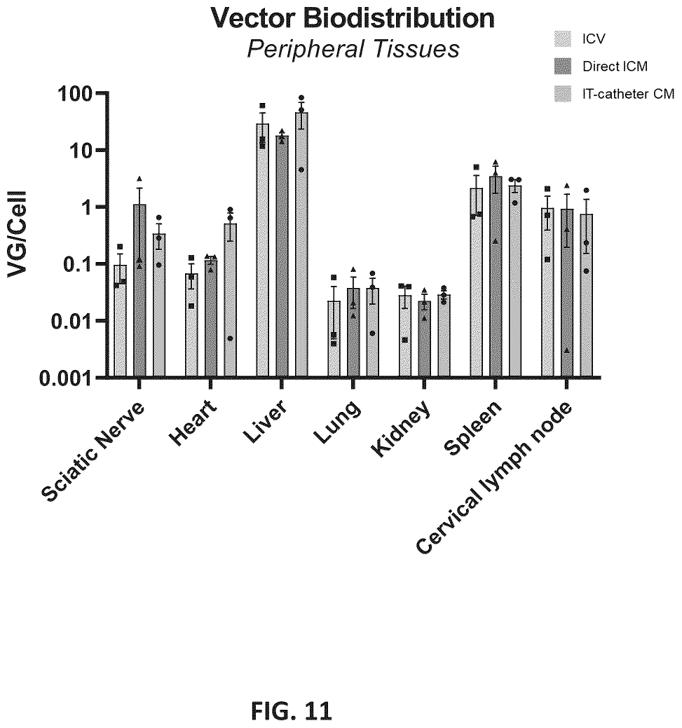

Vector genomes (FIG. 9A) across 20 brain regions indicate broad transduction of AAV.SAN006 with intra-CSF dosing in NHPs. Cortical regions show transduction levels of ˜1VG/cell across ROAs, with ICV dosing yielding a trend toward increased transduction of subcortical structures including caudate and putamen. nLuc activity (FIG. 9B) indicates robust transgene expression across the brain with all three ROAs, with the greatest activity observed across cortical and subcortical regions with ICV dosing. AAV.SAN006 also shows widespread vector biodistribution (FIGS. 10A-B) and transgene activity (FIG. 10C-D) in spinal cord and DRG, with the greatest nLuc activity observed with ICV dosing compared to cisterna magna approaches. AAV.SAN006 also widely transduced peripheral tissues (FIG. 11), with vector detected in sciatic nerve, heart, liver, lung, kidney, spleen, and cervical lymph node with all three intra-CSF ROAs.

From this data, sufficient biodistribution of AAV to disease-relevant CNS structures in NHP with intra-CSF administration can be observed (FIGS. 9A-10D).

AAV.SAN006 (SAN006) delivery intra-CSF improves transgene expression in cerebral cortex while still retaining biodistribution to other relevant organs (heart, lungs, kidney, liver) (FIGS. 9A-11).

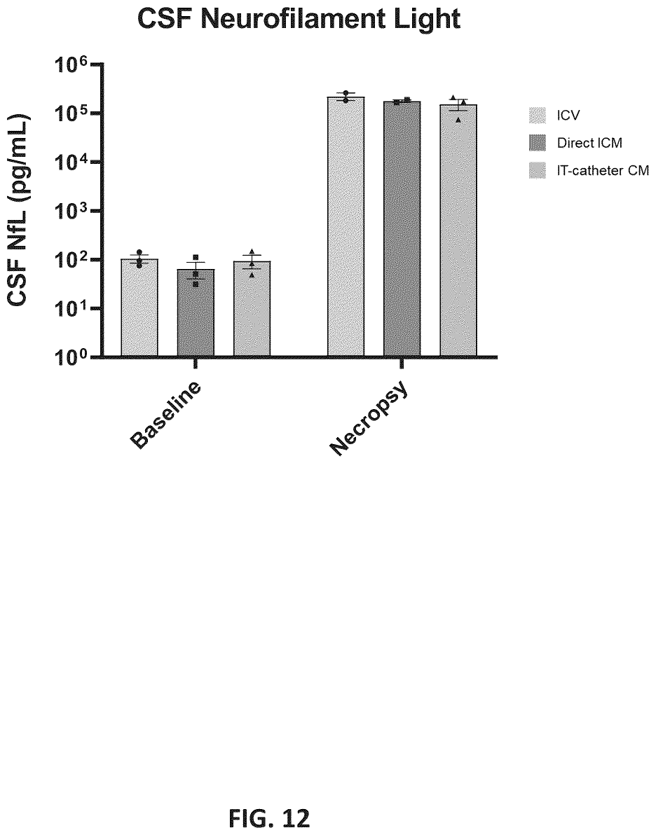

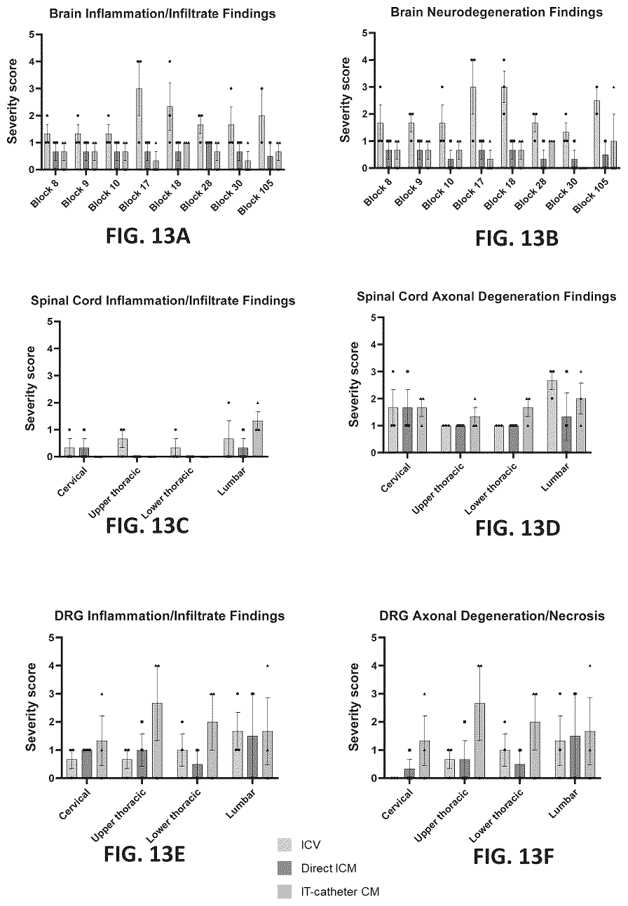

Neurofilament light chain (NfL) measurements in CSF (FIG. 12) show an expected increase from pre-study to the four-week necropsy timepoint that is equivalent across ROAs. Histopathological analyses in brain tissue show minimal to moderate inflammatory (FIG. 13A) and degenerative (FIG. 13B) findings for all intra-CSF ROAs, with marked findings observed only in the ICV group. Minimal to mild inflammatory findings (FIG. 13C) and minimal to moderate degenerative findings (FIG. 13D) were observed in the spinal cord for all ROA. Minimal to moderate inflammatory (FIG. 13E) and degenerative (FIG. 13F) findings were observed in the DRGs across all ROAs, with severe findings only observed in the IT-CM group. No severe inflammatory or neurodegenerative findings were observed in brain, spinal cord, or DRG with AAV.SAN006 by any intra-CSF ROA.

### Example 3: AAV.SAN006 Biodistribution by Intravenous Administration Route

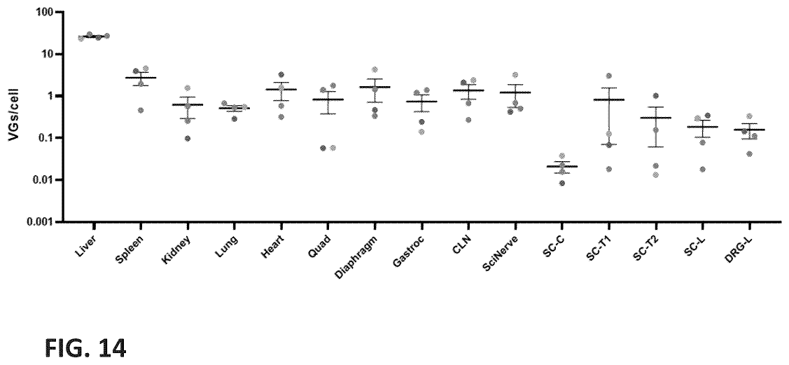

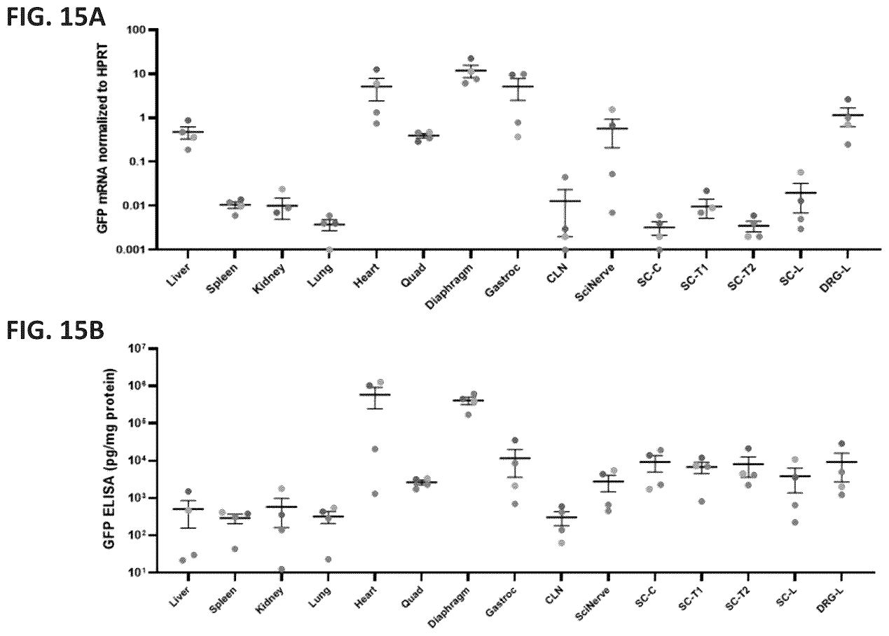

Cynomolgus monkeys (female, aged 2-4 years) seronegative for AAV.SAN006 were dosed with 2.5e13 vg/kg of AAV.SAN006-CBA-eGFP. The virus was injected via intravenous dosing into the saphenous vein, at a dosing volume of 5 ml/kg and infusion rate of 1 ml/min. 3 weeks post-dosing, animals were euthanized and tissues were collected and frozen for subsequent downstream analysis. Vector genomes (FIG. 14) across multiple peripheral tissues indicate broad transduction of AAV.SAN006 with IV dosing in NHPs. GFP mRNA transcript levels (FIG. 15A) across tissues surveyed reveal broad transgene expression not only in major peripheral organs such as liver, kidney and lung but also in muscles such as heart, diaphragm, quadriceps and gastrocnemius. Corresponding GFP ELISA (FIG. 15B) across the same tissues shows the protein expression tracks similar to the mRNA transcript data. All data represented as Mean+SEM. Each data point is tissue from 1 NHP, total of 4 NHPs in the study. Collectively these data indicate that AAV.SAN006 is a suitable capsid for not only CNS targeting with iCM administration but also for targeting peripheral organs with IV dosing.

Liver, spleen and muscle are some of the major organs implicated in GD1. Robust transduction of multiple peripheral tissues such as liver and spleen by AAV.SAN006 IV dosing is demonstrated in FIGS. 14-15B.

Biodistribution to affected tissues with intra-CSF is demonstrated in FIGS. 9A-10D and/or IV administration is demonstrated in FIGS. 14-15B.

### Example 4: Artificial microRNA Sequences Lower MAPT In Vivo Following AAV.SAN006-Mediated Intraparenchymal Delivery

Two amiRNA sequences targeting the human MAPT mRNA, amiRNA1 and amiRNA2, were evaluated in the Tau22 mouse model. Tau22 animals overexpress the human 1N4R tau isoform with two mutations, G272V and P301S, under the neuron-specific Thy 1.2 promoter. These animals exhibit a progressive accumulation of phosphorylated tau isoforms and fibrillar aggregates, with associated neurodegeneration, gliosis, and behavioral deficits (Schindowski et al., 2006). AAV.SAN006-amiRNA vectors encoding either amiRNA1 or amiRNA2 were administered via intraparenchymal injection to the striatum of two-month-old Tau22 transgenic mice. After one month, human MAPT mRNA was significantly reduced by over 50% in the striatum of amiRNA2-treated animals. amiRNA2-trateated animals showed a trend toward 40% knockdown.

Furthermore, artificial miRNA sequences reduce expression of the human MAPT mRNA encoding tau in a mouse model of tauopathy following AAV.SAN006-miRNA injection.

### Example 5: Artificial microRNA Sequences Lower HTT In Vivo Following AAV.SAN006-Mediated Intraparenchymal Delivery

Select candidate amiRNAs were assessed in vivo in rodent models of HD. The YAC128 mouse (Slow et al, 2003) and BACHD rat (Yu-Taeger et al, 2012) are well characterized HD lines that each express full-length human mutant HTT and are therefore suitable for assessing in vivo target engagement. In each of these strains, five different amiRNAs were delivered via intraparenchymal injection to the striatum using the novel capsid AAV.SAN006 (8E10 particles per YAC128 mouse, 1E11 particles per BACHD rat). After six weeks, all tested amiRNAs generated significant striatal knockdown of human HTT mRNA in the YAC128 model and, importantly, significant reduction of mutant HTT protein was observed in both rodent models for all tested HTT-targeting amiRNAs.

Furthermore, artificial miRNA sequences reduce human HTT protein levels in two different models of Huntington's disease following AAV.SAN006-miRNA injection.

### Example 6: Artificial microRNA Sequences Lower SNCA In Vivo Following AAV.SAN006-Mediated Intraparenchymal Delivery

In this example, candidate amiRNAs were evaluated for their ability to reduce human SNCA in vivo. For in vivo studies transgenic mice were used that express the entire human SNCA gene including UTRs, enabling evaluation of guides which target the 3′UTR of SNCA mRNA. AAV.SAN006 vectors were administered directly into the striatum at 3e10 VG per hemisphere. Total striatal RNA and genomic DNA was isolated to evaluate target reduction and compared to control animals injected with formulation buffer. Several amiRNAs exhibited evidence of potent SNCA mRNA reduction.

Additionally, artificial miRNA sequences reduce human SNCA mRNA levels in vivo in transgenic mice expressing human SNCA following AAV.SAN006-miRNA injection.

### Example 7: AAV.SAN006-SS3-GBA1 Mediated Gene Therapy for the Treatment of Gaucher Disease and GBA-PD

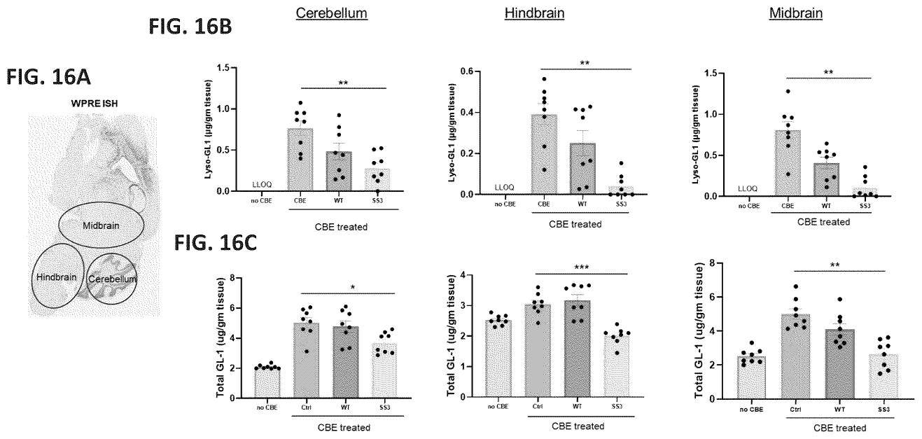

Efficient substrate clearance by SS3-GBA1 variant in vivo in rodent brain tissues: To demonstrate efficacy of the lead GBA1 variant SS3-GBA1 (SEQ ID NO: 23) (see Example 8) in comparison to WT GBA1 (SEQ ID NO: 19), AAV.SAN006-WT GBA1 or AAV.SAN006-SS3-GBA1 (1e11 VGs) was injected in WT mice via bilateral ICV injections. WT GBA1 (SEQ ID NO: 19) encodes an enzyme (GCase) having an amino acid sequence of SEQ ID NO: 17. Following cleavage of the signaling peptide (amino acids 1-39), the resultant GCase enzyme has a an amino acid sequence of SEQ ID NO: 18. The viruses were allowed to express for 4 weeks, and 24 hours prior to necropsy lipid accumulation was induced by injected mice with 100 mg/kg CBE (conduritol β-epoxide, IP injection). The brain was micro-dissected into smaller regions (FIG. 16A) and assessed Lyso-GL1 and GL1 levels via lipidomics mass spectrometry. Under physiological conditions, Lyso-GL1 levels in WT mice (no CBE group) are undetectable or LLOQ (lower limit of quantification) and total GL1 levels are about 2-3 μg/gm tissue weight, depending on the specific brain region. Across all regions surveyed a spike in lipid levels (both Lyso-GL1 and GL1) was observed in the vehicle injected, CBE treated group demonstrating the action of CBE to induce lipid accumulation by inhibiting GCase. Surveying both lyso-GL1 (FIG. 16B) and GL1 (FIG. 16C), SS3-GBA1 was observed effectively reducing lipid accumulation. This construct was highly efficacious as seen by some mice that had LLOQ Lyso-GL1 levels in their hindbrain and midbrain indicating effective clearance of the accumulated lipids.

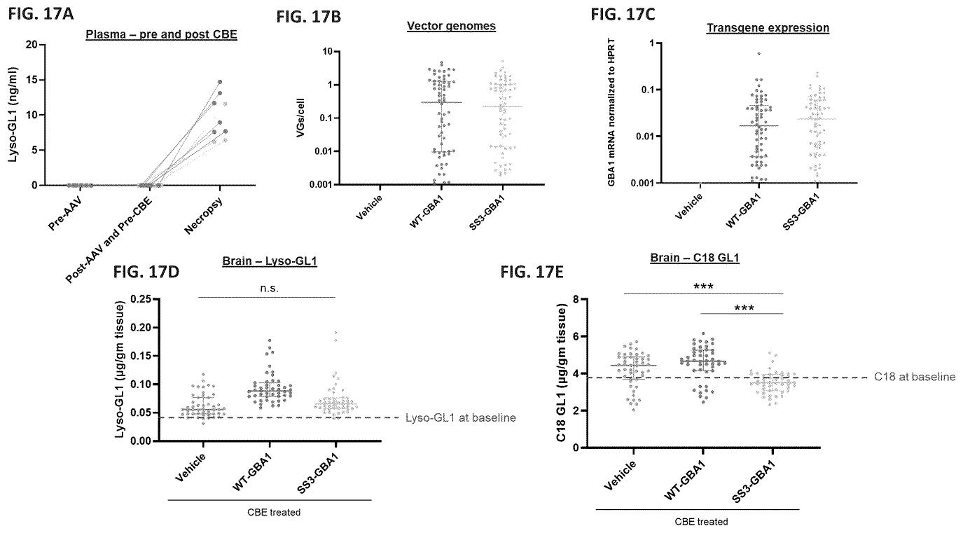

Effective reduction of GL1 lipids in SS3-GBA1 treated NHPs: A pharmacology study in Cynomolgus NHPs was also performed to determine efficacy of AAV.SAN006-SS3-GBA1 in CBE-treated NHPs. Either WT-GBA1 or SS3-GBA1 AAVs were injected in 2-3 yr old NHPs (1.25e13 VGs/NHP) via intra-cisterna magna (intra-CSF) dosing. The virus was allowed to express for 6 weeks at which point all NHPs were injected with 30 mg/kg CBE intravenously 48 hours prior to necropsy. Plasma was collected at various timepoints such as pre-AAV, pre-AAV and pre-CBE, and post-CBE at necropsy. A spike in Lyso-GL1 was observed in the plasma of all CBE-treated NHPs at necropsy (FIG. 17A).

Vector genomes (FIG. 17B) and transgene expression of huGBA1 mRNA (FIG. 17C) was comparable across two virus treated groups (WT-GBA1 in red and SS3-GBA1 in beige). Data is presented as median with inter-quartile range; each data point is average of all NHPs in that group for that punch. While a decrease in Lyso-GL1 was not observed in virus treated groups compared to vehicle treated in the grey mater punches surveyed (FIG. 17D), SS3-GBA1 was able to significantly reduce the accumulated C18 GL1, a predominant isotype of GL1 in the brain (FIG. 17E). SS3-GBA1 brought back the C18 GL1 levels to almost physiological level, as indicated by the purple baseline. SS3-GBA1 effectively promotes substrate clearance in mouse and NHP model of GBA-PD as depicted in FIGS. 16A-17E).

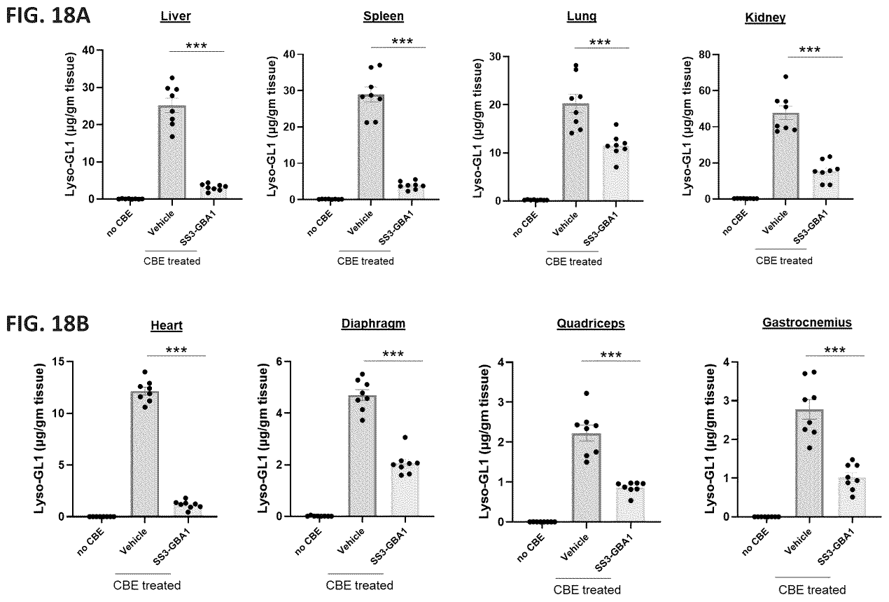

SS3-GBA1 effectively promotes substrate clearance in intravenously dosed WT mice: Based on intra-CSF studies in mice and NHPs comparing efficacy of WT-GBA1 versus SS3-GBA1, SS3-GBA1 was nominated as the lead construct. To facilitate IV dosing of the lead construct for Gaucher disease patients (Type 1), AAV.SAN006-SS3-GBA1 was also investigated if it would be able to clear/reduce accumulated lipids with an IV route of administration. Either vehicle or AAV.SAN006-SS3-GBA1 was injected via IV dosing in 3 month old mice (4e13 VG/kg). AAVs were expressed for 4 weeks followed by 100 mg/kg IP injection of CBE, 24 hours prior to necropsy. Consistent with intra-CSF dosing studies in mice, a robust decrease was observed in Lyso-GL1 across all major peripheral organs (FIG. 18A) as well as all muscles surveyed (FIG. 18B). This establishes the efficacy of AAV.SAN006 SS3-GBA1 across 2 different routes of administration in vivo in mice.

### Example 8. Payload Engineering Strategy to Generate GBA1 Variants that are Readily Secretable

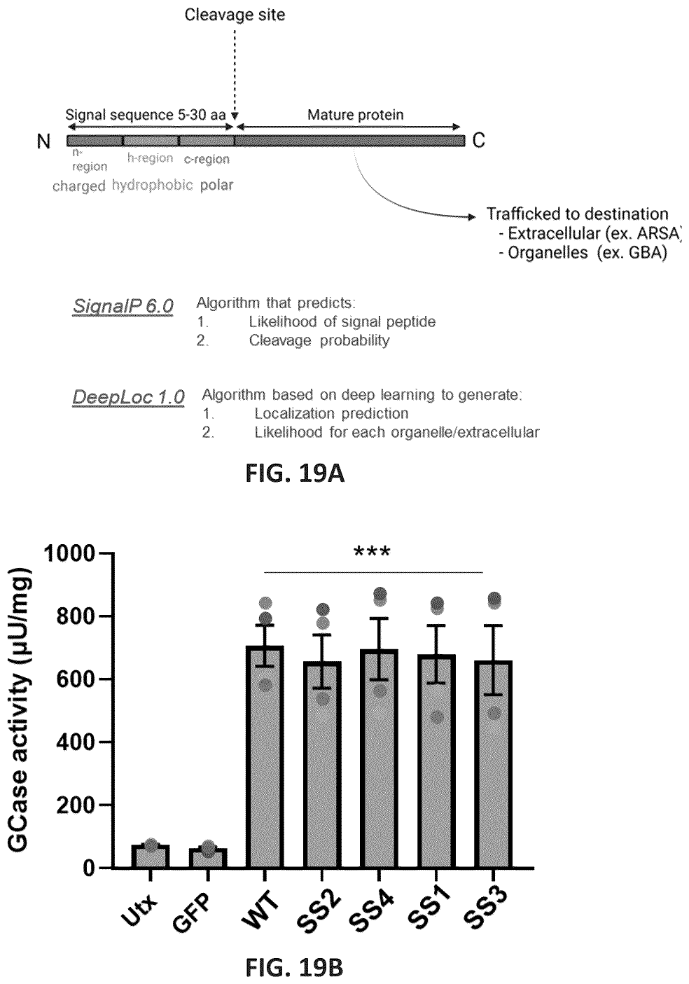

Human GBA1 (NP_000148.2) was engineered to make it readily secretable. To do this, the endogenous signal sequence at the N-terminus of GBA1 protein was replaced with signal sequences of some highly secreted proteins. Using a combination of in silico tools, 4 such signal sequences (SS1 (SEQ ID NO: 20), SS2 (SEQ ID NO: 21), SS3 (SEQ ID NO: 23), and SS4 (SEQ IDNO: 24)) were identified that yielded the highest probability of being secreted extracellularly and also had the highest probability (>98%) of the signal sequence being cleaved (FIG. 19A). All sequences were human codon-optimized and driven by the hybrid CMV-chicken β-actin (CBA) constitutive promoter. Transgene expression was further enhanced by the addition of the woodchuck hepatitis virus post-transcriptional regulatory element (WPRE). Detailed vector maps of all constructs are provided.

To ensure GCase activity is not compromised with the swapped signal sequences, HEK293 cells were transfected with the different GBA1 constructs and examined GCase activity in cell lysates 48-hour post transfections. Significantly increased GCase enzyme activity was observed in WT GBA1 transfected HEK cells compared to untransfected cells. Further, all the GBA1 variants that were generated had enzyme activity comparable to that of WT GBA1. No statistical differences were observed within the different GBA1 constructs (FIG. 19B).

### Example 9: Robust Secretion of Some GBA1 Variants In Vivo in Rodent Brain Tissues

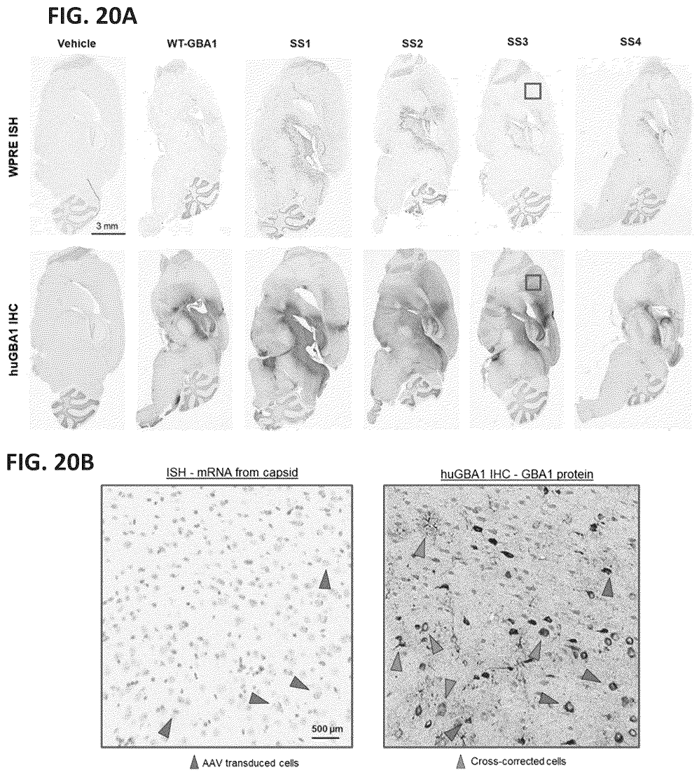

To investigate the ability of engineered constructs to secrete and be taken up by non-AAV transduced cells in vivo, bilateral ICV (intra-cerebroventricular) injections of AAVs (1e11 VG/mouse, 5 μl per hemisphere) were performed in 4 month old WT mice and analyzed brain sections after 4 weeks of AAV expression. In situ hybridization against the 3′ UTR of the transgene (WPRE) were performed to evaluate distribution of AAV-transduced cells and in adjacent fixed sections, performed immunohistochemistry for human GBA1 protein (FIG. 20A). While the virus was largely localized to regions around the ventricles in the sagittal sections of all AAV treatment groups, a much larger spread of the huGBA1 protein in SS1, SS2, SS3 was observed compared to the WT GBA1. Higher magnification imaging of these sections revealed that not only was there robust secretion of the engineered GBA1, in this case SS3-GBA1 (FIG. 20B), but also efficient uptake of the secreted protein was observed in a variety of cell types in the brain as seen in the morphological differences of cells taking up the secreted protein (marked by green arrows).

### Example 10: Efficient Substrate Clearance by SS3-GBA1 Variant In Vivo in Rodent Brain Tissues

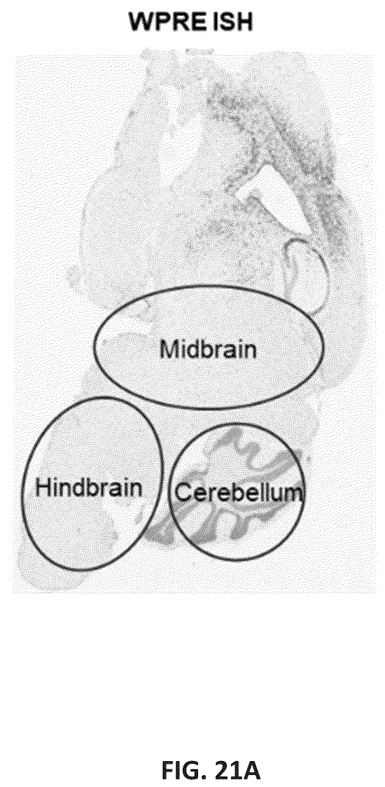

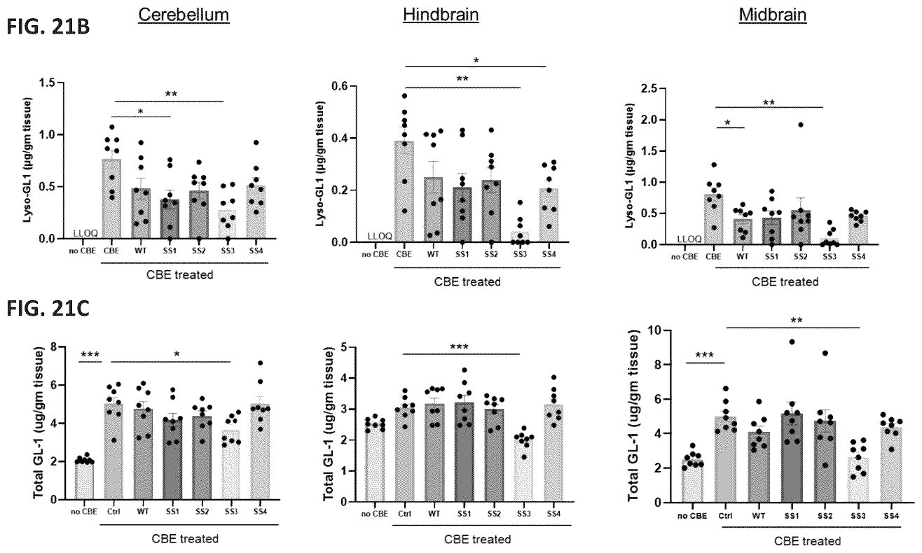

To demonstrate efficacy of GBA1 variants the indicates AAVs (1e11 VGs) were injected into WT mice for 4 week of expression time and induced lipid accumulation by injecting them with 100 mg/kg CBE (conduritol β-epoxide, IP injection) 24 hours prior to necropsy. The brain was micro-dissected into smaller regions (FIG. 21A) Lyso-GL1 and GL1 levels were assessed via lipidomics mass spectrometry. Under physiological conditions, Lyso-GL1 levels in WT mice (no CBE group) are undetectable or below the LLOQ (lower limit of quantification) and total GL1 levels are 2-3 μg/gm tissue weight, depending on the specific brain region (grey bars in FIG. 21C). Across all regions surveyed a significant increase in lipid levels (both Lyso-GL1 and GL1) was observed in the CBE treated, vehicle injected group demonstrating the action of CBE to induce lipid accumulation by inhibiting GCase. Surveying both Lyso-GL1 (FIG. 21B) and GL1 (FIG. 21C), SS3-GBA1 effectively reducing lipid accumulation was observed. This construct outperformed all the other constructs generated and in a few mice, the increased Lyso-GL1 with CBE reverted back to LLOQ as seen with datapoints plotted as 0 in the hindbrain and midbrain tissues.

### Example 11: Development of CBE-Induced Lipid Flux Model in Non-Human Primates (NHPs)

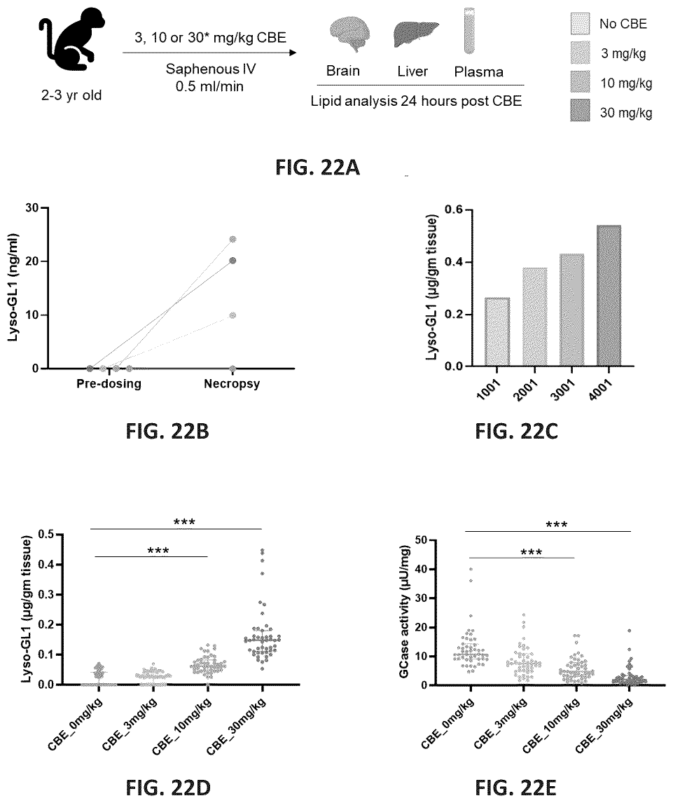

To develop an efficacy model in NHPs, the CBE model developed in rodents and was adapted for NHPs to determine CBE-dosing regimen. Cynomolgus monkeys (Mauritian, 2-3 yr old) were injected with 3, 10 or 30 mg/kg CBE intravenously via the saphenous vein Brains, liver and plasma were collected for lipid analyses 24 hours post CBE-dosing (FIG. 22A).Lyso-GL1 levels was detectable in the plasma of all NHPs that got CBE (FIG. 22B) and a dose-dependent increase in Lyso-GL1 was observed in liver from these NHPs (FIG. 22C). For analysis in brains, 47 gray matter brain punches which correspond to 17 different grey mater regions were surveyed. Consistent with the liver data, a dose-dependent increase in Lyso-GL1 was observed across the 4 treatment groups with 10 mg/kg and 30 mg/kg yielding a significant increase in Lyso-GL1 levels compared to the “No CBE” group (FIG. 22D). Furthermore, a concomitant decrease in the GCase activity was observed across all the treatment groups (FIG. 22E) consistent with the mechanism of action of CBE inhibiting GCase enzyme activity.

### Example 12: Effective Reduction of GL1 Lipids in SS3-GBA1 Treated NHPs

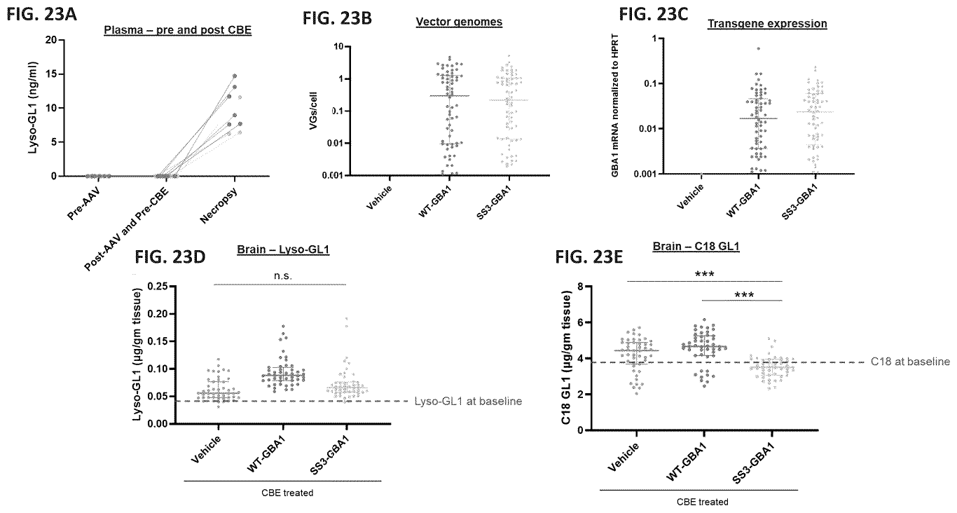

A pharmacology study in Cynomolgus NHPs was performed to determine efficacy of AAVs in CBE-treated NHPs. AAVs (e.g., AAVSAN006 WT GBA1 and AAV.SAN006 SS3-GBA1) were injected in 2-3 yr old NHPs (1.25e13 VGs/NHP) via intra-cisterna magna (intra-CSF) dosing. The viruses were allowed to express for 6 weeks and all NHPs were injected with 30 mg/kg CBE intravenously 48 hours prior to necropsy. Plasma was collected at the indicated timepoints such as pre-AAV, pre-AAV and pre-CBE, and post-CBE at necropsy. A spike in Lyso-GL1 in the plasma of all CBE-treated NHPs was observed at necropsy (FIG. 23A).

Vector genome levels (FIG. 23B) and transgene expression of huGBA1 mRNA (FIG. 23C) were comparable across two virus treated groups (WT-GBA1 in red and SS3-GBA1 in light brown). Data is presented as median with inter-quartile range; each data point is average of all NHPs in that group for that punch. Although a significant decrease in Lyso-GL1 levels of AAV treated NHP was not observed when compared to vehicle injected group (FIG. 23D), SS3-GBA1 was able to significantly reduce the accumulated C18 GL1, a predominant isotype of GL1 in the brain (FIG. 23E). Purple line indicates lipid levels under physiological conditions.

Importantly, AAV.SAN006-SS3-GBA1 treatment lowers the accumulated C18 GL1 to physiological levels as seen by the median of the data close to the purple line which marks the baseline physiological lipid levels. In summary, this study resulted in: a) comparable vector genomes and transgene expression of WT GBA1 and SS3-GBA1 across NHPs; b) increase in lyso-Gl1 in plasma of all NHPs at necropsy, indicative of CBE working effectively; c) statistically significant reduction in C18 GL1 in SS3-GBA1 treated NHPs compared to vehicle and WT-GBA1; and d) histological analyses demonstrate clear secretion and uptake of SS3-GBA1 in non-transduced cells in NHP brain tissues.

### Example 13: Robust Secretion and Diffusion of SS3-GBA1 in Brain Sections of SS3-GBA1 iCM dosed NHPs

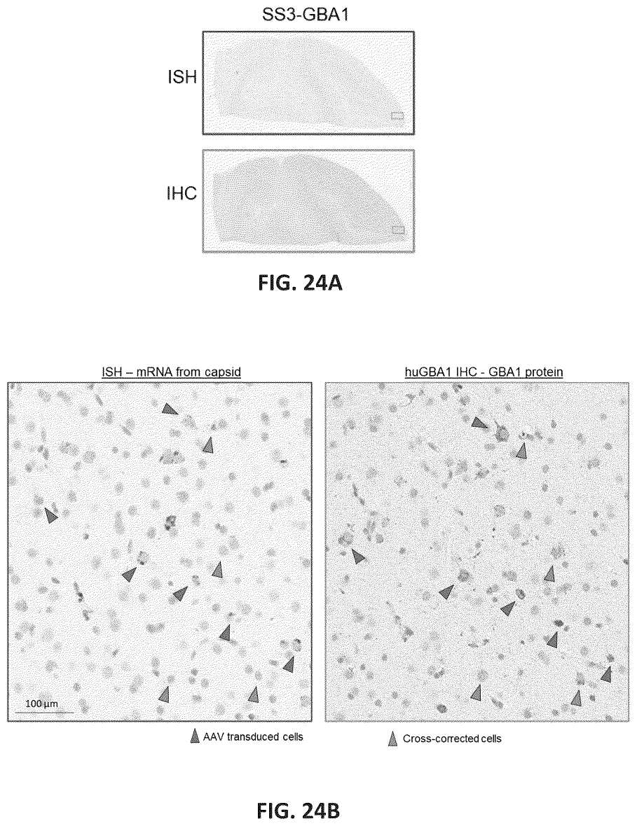

WPRE mRNA was performed in situ hybridization and huGBA1 immunohistochemistry in 5 μm FFPE NHP brain sections from animals were treated with AAV.SAN006-SS3-GBA1 (FIG. 24A). Higher magnification imaging of these images revealed that consistent with previous observations in rodents, SS3-GBA1 was robustly secreted and taken up by non-transduced cells, shown in green arrows (FIG. 24B).

### Example 14: NHP Pharmacology and Dose Range Finding Study Evaluating AAVSAN006-ARSA

A codon-optimized transgene (SEQ ID NO:2) encoding the ARSA polypeptide (ARSA) (SEQ ID NO: 1) was generated. An expression vector comprising the codon-optimized transgene (SEQ ID NO:2) was encapsulated in an rAAV viral particle comprising the AAVSAN006 VP1, VP2 and VP3 capsid proteins. SEQ ID NO: 3 shows the full DNA sequence of the plasmid encoding ARSA for rAAV packaging;

### Methods

Briefly, purpose bred, naive, male/female cynomolgus (Cambodia 2-3 yr old, 2.6-3.1 kg) NHPs seronegative for AAVSAN006 neutralizing antibodies were dosed by single direct cisterna magna (ICM) infusion. Animals were placed in Trendelenburg position during dosing. The dosing paradigm involved a single 2.5 mL infusion of AAVSAN006-ARSA (LOT #VP050322) at 0.125 ml/min, followed by a 250 μl flush with formulation buffer. Five weeks post-dosing, animals were euthanized, and samples were assessed for vector biodistribution (dPCR) and hARSA mRNA (RTdPCR), accompanied by a safety assessment. Samples represent 64 punches from brain representing 19 distinct grey matter regions and 7 distinct white matter regions, 8 segments of the spinal cord with adjacent DRGs, peripheral nerves and visceral organs. The study design is described in Table 1.

TABLE 1
Study CRL.2021-5613: NHP dose range finding study

| | | Dose/gm | | Dosing | | Time points, sample |
|---|---|---|---|---|---|---|
| Group | Test article | brain weight | Dose/animal | regimen | Animal #'s | collection, analysis |
| 1 | AAVSAN006- | 1e10 VG | 7.5e11 VG | RoA: ICM with | 5 (M/F) | In-life: 35 days |
| 2 | ARSA | 3.3e10 VG | 2.5e12 VG | animal placed | 5 (M/F) | Brain, spinal cord, |
| 3 | | 1e11 VG | 7.5e12 VG | in | 5 (M/F) | DRG, peripheral |
| 4 | | 3.3e11 VG | 2.5e13 VG | Trendelenburg | 5 (M/F) | nerves, visceral organs |
| 5 | Formulation | n/a | n/a | position | 3 (M/F) | at necropsy |
| | Buffer | | | Doing | | CSF (pre-study, and at |
| | | | | parameters: | | necropsy) |
| | | | | 2.5 ml | | Plasma: pre-study, days |
| | | | | @0.125 mL/min | | post-dose: 2, 4, 7, 14 |
| | | | | with 250 ul | | and at necropsy |
| | | | | flush | | Neurological/behavioral |
| | | | | | | assessment (pre-dose, |
| | | | | | | 7-day and 5 wk post- |
| | | | | | | dose) |

### Results

A dose-dependent increase in AAVSAN006-ARSA vector biodistribution was observed in NHP brain with ICM dosing, both in grey (19 unique brain regions) and white matter regions (7 unique brain regions). AAVSAN006-ARSA treatment also resulted in wide-spread dose-dependent increase in hARSA mRNA and protein levels at the two top doses: 7.5e12VG (1e11VG/gm brain weight) and 2.5e13VG (3.3e11VG/gm brain weight). Uniform, dose-dependent vector biodistribution and hARSA expression was observed in DRGs and spinal cord, along the spinal rostral-caudal axis. Among the visceral organs, liver, spleen, and cervical lymph node show a dose-dependent increase vector biodistribution.

To explore whether administration of AAVSAN006-ARSA led to meaningful human ARSA expression in NHP brains, we assessed the level of AAV-derived human ARSA protein and compared to 1) endogenous cynomolgus cyARSA protein (hARSA/cyARSA in 19 brain regions measured within the same samples) and to 2) human ARSA protein measured in the brains (in 12 brain regions) of 7 healthy human organ-donors between the ages of 3 and 8 years old. At doses of 1e11VG/gm and 3.3e11VG/gm brain weight, brain-wide mean human ARSA protein levels were ˜63% and ˜546% of native cyARSA. In addition, at doses of 1e11VG/gm and 3.3e11VG/gm brain weight, brain-wide mean human ARSA protein levels were ˜51% and ˜416% of human ARSA protein levels from healthy control organ-donors. Therefore, AAVSAN006-ARSA treatment in NHPs results in therapeutically meaningful ARSA protein expression in the CNS at 1e11 and 3.3e11VG/gm brain weight.

No clinical signs (functional or behavioral deficits) were observed in NHPs at week 1 or week 5 (necropsy) post-dosing in either dosing groups, compared to pre-dose tests. Intra-CM infusion was well tolerated; as expected, the ICM procedure accounted for a significant increase in CSF Nf-L levels, with further increase observed in AAVSAN006-ARSA treated NHPs compared to formulation buffer treated NHPs. However, no dose-dependent increase was observed. No significant change in plasma cytokine concentration were observed in NHPs treated with AAVSAN006-ARSA across any dose, and no cell-mediated immune response was noted to AAVSAN006 capsid or hARSA protein in NHPs treated with AAVSAN006-ARSA at the two high doses by IFN-γ ELISpot. Furthermore, no test item-related gross finding noted with any animal at necropsy.

Cerebrospinal fluid changes consisted of minimal, non-dose-related, increases in nucleated cell counts (primarily of mononuclear cells) in most animals from 1e10 VG/gm brain weight. In addition, albumin and/or total proteins were increased in most animals at ≥3.3e10 VG/gm brain weight at 5-weeks post vector administration. A few individual animals at 3.3e10 or 3.3e11 VG/gm brain weight had variable amount of basophilic to eosinophilic, granular material morphologically compatible with neural material. These changes correlated with neuronal degeneration and mononuclear infiltration noted in various sections from the central nervous system.

Hematology changes consisted of transient mild increases in reticulocyte count in most males ≥ 1e10 VG/gm brain weight and in a few sporadic females at 1e11 VG/gm brain weight on Day 7 with concurrent minimal decreases in red blood cell mass parameters in a few individuals. There were increases in white blood cell counts secondary to increases in neutrophil, lymphocyte, and/or monocyte counts in some males and females at 3.3e10 VG/gm brain weight, still noted in some animals, mainly in males administered 3.3e11 VG/gm brain weight. No test-article related effects were noted on coagulation or clinical chemistry.

The main test article effects were comprised of neuronal degeneration (affecting brain, lumbar spinal cord, and DRG), increase glial cell response (cerebellum, and spinal cord), nerve fiber degeneration (affecting the white matter of spinal cord and nerve fibers of peripheral nerves), and mononuclear cell infiltrate (including perivascular distribution) which affected the brain (cerebellum), DRG, and spinal cord. No test article related findings were observed in visceral organs (Heart, liver, gallbladder, spleen, pancreas, adrenal gland, lung, bone, sternum/marrow, ovary, duodenum, testis, epididymis, thymus, eye, uterus with cervix, kidney) at any dose. A dose response was noted between the lowest dose (1e10VG/gm brain weight) and the highest dose (3.3e11VG/gm brain weight) for some changes in some locations such as neuronal degeneration (cerebellum, lumbar DRGs at the highest dose), gliosis (cerebral cortex, cerebellum, but not in the spinal cord), and for mononuclear cell infiltrates (in the cerebral cortex and in lumbar DRGs at the highest dose).

In conclusion, single administration of AAVSAN006-ARSA by direct intra-cisterna magna (ICM) was well tolerated in cynomolgus monkeys (both male and female) at dosing levels of 1e10, 3.3e10, 1e11 and 3.3e11 VG/gm brain weight, and 1e11 and 3.3e11 VG/gm brain weight were deemed efficacious doses.

```
SEQUENCES
ARSA Polypeptide Sequence
(SEQ ID NO: 1)
                10 20 30 40 50
MGAPRSLLLA LAAGLAVARP PNIVLIFADD LGYGDIGCYG HPSSTTPNLD
                60 70 80 90 100
QLAAGGIRFT DFYVPVSLCT PSRAALLTGR LPVRMGMYPG VLVPSSRGGL
              110 120 130 140 150
PLEEVTVAEV LAARGYLTGM AGKWHLGVGP EGAFLPPHQG FHRFLGIPYS
              160 170 180 190 200
HDQGPCQNLT CFPPATPCDG GCDQGLVPIP LLANLSVEAQ PPWLPGLEAR
              210 220 230 240 250
YMAFAHDLMA DAQRQDRPFF LYYASHHTHY PQFSGQSFAE RSGRGPFGDS
              260 270 280 290 300
IMELDAAVGT IMTAIGDEGL LEETLVIFTA DNGPETMRMS RGGCSGLLRC
              310 320 330 340 350
GKGTTYEGGV REPALAFWPG HIAPGVTHEL ASSLDLLPTL AALAGAPLPN
              360 370 380 390 400
VILDGFDLSP LLLGTGKSPR QSLFFYPSYP DEVRGVFAVR TGKYKAHFFT
              410 420 430 440 450
QGSAHSDTTA DPACHASSSL TAHEPPLLYD LSKDPGENYN LLGGVAGATP
              460 470 480 490 500
EVIQALKQLQ LLKAQLDAAV TFGPSQVARG EDPALQICCH PGCTPRPACC
HCPDPHA
Codon Optimized ARSA DNA sequence (Human)
(SEQ ID NO: 2)
ATGAGCATGGGAGCCCCTAGATCTCTGCTGCTGGCTCTTGCTGCTGGACTGGCTGTGGCCAGA
CCTCCTAACATCGTGCTGATCTTCGCCGACGATCTCGGCTATGGCGATCTGGGCTGTTACGGA
CACCCTAGCAGCACCACACCTAACCTGGATCAACTGGCTGCCGGCGGACTGAGATTCACCGAT
TTCTACGTGCCCGTGTCTCTGTGCACACCTAGTAGAGCTGCTCTGCTGACAGGCAGACTGCCA
GTGCGGATGGGAATGTATCCTGGCGTGCTGGTTCCTAGCAGTAGAGGCGGACTGCCTCTGGAA
GAAGTGACAGTTGCTGAAGTGCTGGCCGCCAGAGGCTATCTGACTGGAATGGCCGGAAAATGG
CACCTCGGAGTTGGACCTGAAGGCGCTTTTCTGCCTCCTCACCAGGGCTTCCACAGATTTCTG
GGCATCCCTTACAGCCACGATCAGGGCCCTTGCCAGAACCTGACCTGCTTTCCTCCTGCCACA
CCTTGTGATGGCGGCTGTGATCAGGGACTCGTGCCTATTCCTCTGCTGGCCAATCTGAGCGTG
GAAGCTCAACCTCCTTGGCTGCCTGGCCTGGAAGCCAGATATATGGCCTTCGCTCACGACCTG
ATGGCCGACGCTCAGAGACAGGACAGACCATTCTTCCTGTACTACGCCAGCCACCACACACAC
TACCCTCAGTTCTCTGGCCAGTCCTTCGCCGAGAGATCTGGCAGAGGCCCTTTTGGCGATAGC
CTGATGGAACTGGATGCCGCCGTGGGAACACTGATGACAGCCATTGGAGATCTGGGCCTGCTG
GAAGAGACACTGGTCATCTTCACCGCCGACAACGGCCCCGAGACAATGAGAATGAGCAGAGGC
GGCTGTAGCGGCCTGCTGAGATGTGGCAAGGGAACAACATACGAAGGCGGCGTCAGAGAGCCT
GCTCTGGCTTTTTGGCCTGGACATATTGCCCCTGGCGTGACACACGAACTGGCCTCTTCTCTG
GATCTGCTGCCTACACTGGCTGCTTTGGCTGGCGCTCCTCTGCCTAATGTGACCCTGGATGGC
TTCGATCTGTCTCCACTGCTGCTCGGAACAGGCAAGAGCCCTAGACAGAGCCTGTTCTTCTAC
CCTAGCTACCCCGATGAAGTGCGGGGAGTGTTTGCCGTGCGGACAGGCAAGTACAAGGCCCAC
TTTTTTACCCAAGGCAGCGCCCACAGCGATACCACAGCTGATCCTGCTTGTCACGCCTCTAGC
AGCCTGACAGCTCATGAACCACCTCTGCTGTACGACCTGTCTAAGGACCCCGGCGAGAACTAT
AATCTGCTTGGCGGAGTTGCCGGCGCTACACCTGAAGTTCTGCAGGCTCTGAAACAGCTCCAG
CTGCTGAAAGCCCAGCTGGACGCTGCTGTGACATTTGGACCTTCTCAGGTGGCAAGAGGCGAG
GACCCTGCTCTGCAGATTTGTTGTCACCCTGGCTGTACCCCTAGACCTGCCTGCTGTCACTGT
CCTGATCCTCACGCTTGA
Full DNA sequence of plasmid for rAAV packaging; coding sequence
for ARSA is underlined
(SEQ ID NO: 3)
TTGGCCACTCCCTCTCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGCCAAC
TCCATCACTAGGGGTTCCTTACGTACAATTGGGATCCCGGACCGTCGACATTGATTATTGACT
AGTTATTAATAGTAATCAATTACGGGGTCATTAGTTCATAGCCCATATATGGAGTTCCGCGTT
ACATAACTTACGGTAAATGGCCCGCCTGGCTGACCGCCCAACGACCCCCGCCCATTGACGTCA
ATAATGACGTATGTTCCCATAGTAACGCCAATAGGGACTTTCCATTGACGTCAATGGGTGGAG
TATTTACGGTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTCGAGGTGAGCCCC
ACGTTCTGCTTCACTCTCCCCATCTCCCCCCCCTCCCCACCCCCAATTTTGTATTTATTTATT
TTTTAATTATTTTGTGCAGCGATGGGGGCGGGGGGGGGGGGGGGGCGCGCGCCAGGCGGGGCG
GGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGC
GCGCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCCCT
TCTCCTCCGGGCTGTAATTAGCGCTTGGTTTAATGACGGCTTGTTTCTTTTCTGTGGCTGCGT
GAAAGCCTTGAGGGGCTCCGGGAGGGCCCTTTGTGCGGGGGGAGCGGCTCGGGGGGTGCGTGC
GTGTGTGTGTGCGTGGGGAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGGAGCGCGGCCGGGGGCGGT
GCCCCGCGGTGCGGGGGGGGCTGCGAGGGGAACAAAGGCTGCGTGCGGGGTGTGTGCGTGGGG
GGGTGAGCAGGGGGTGTGGGCGCGTCGGTCGGGCTGCAACCCCCCCTGCACCCCCCTCCCCGA
GTTGCTGAGCACGGCCCGGCTTCGGGTGCGGGGCTCCGTACGGGGCGTGGCGCGGGGCTCGCC
GTGCCGGGCGGGGGGGGCGGCAGGTGGGGGTGCCGGGGGGGGCGGGGCCGCCTCGGGCCGGGG
AGGGCTCGGGGGAGGGGCGCGGCGGCCCCCGGAGCGCCGGCGGCTGTCGAGGCGCGGCGAGCC
GCAGCCATTGCCTTTTATGGTAATCGTGCGAGAGGGCGCAGGGACTTCCTTTGTCCCAAATCT
GTGCGGAGCCGAAATCTGGGAGGCGCCGCCGCACCCCCTCTAGCGGGCGCGGGGCGAAGCGGT
GCGGCGCCGGCAGGAAGGAAATGGGCGGGGAGGGCCTTCGTGCGTCGCCGCGCCGCCGTCCCC
TTCTCCCTCTCCAGCCTCGGGGCTGTCCGCGGGGGGACGGCTGCCTTCGGGGGGGACGGGGCA
GGGGGGGGTTCGGCTTCTGGCGTGTGACCGGCGGCTCTAGAGCCTCTGCTAACCATGTTCATG
CCTTCTTCTTTTTCCTACAGCTCCTGGGCAACGTGCTGGTTATTGTGCTGTCTCATCATTTTG
GCAAAGAATTCTACGTACCACCATGAGCATGGGAGCCCCTAGATCTCTGCTGCTGGCTCTTGC
TGCTGGACTGGCTGTGGCCAGACCTCCTAACATCGTGCTGATCTTCGCCGACGATCTCGGCTA
TGGCGATCTGGGCTGTTACGGACACCCTAGCAGCACCACACCTAACCTGGATCAACTGGCTGC
CGGCGGACTGAGATTCACCGATTTCTACGTGCCCGTGTCTCTGTGCACACCTAGTAGAGCTGC
TCTGCTGACAGGCAGACTGCCAGTGCGGATGGGAATGTATCCTGGCGTGCTGGTTCCTAGCAG
TAGAGGCGGACTGCCTCTGGAAGAAGTGACAGTTGCTGAAGTGCTGGCCGCCAGAGGCTATCT
GACTGGAATGGCCGGAAAATGGCACCTCGGAGTTGGACCTGAAGGCGCTTTTCTGCCTCCTCA
CCAGGGCTTCCACAGATTTCTGGGCATCCCTTACAGCCACGATCAGGGCCCTTGCCAGAACCT
GACCTGCTTTCCTCCTGCCACACCTTGTGATGGCGGCTGTGATCAGGGACTCGTGCCTATTCC
TCTGCTGGCCAATCTGAGCGTGGAAGCTCAACCTCCTTGGCTGCCTGGCCTGGAAGCCAGATA
TATGGCCTTCGCTCACGACCTGATGGCCGACGCTCAGAGACAGGACAGACCATTCTTCCTGTA
CTACGCCAGCCACCACACACACTACCCTCAGTTCTCTGGCCAGTCCTTCGCCGAGAGATCTGG
CAGAGGCCCTTTTGGCGATAGCCTGATGGAACTGGATGCCGCCGTGGGAACACTGATGACAGC
CATTGGAGATCTGGGCCTGCTGGAAGAGACACTGGTCATCTTCACCGCCGACAACGGCCCCGA
GACAATGAGAATGAGCAGAGGCGGCTGTAGCGGCCTGCTGAGATGTGGCAAGGGAACAACATA
CGAAGGCGGCGTCAGAGAGCCTGCTCTGGCTTTTTGGCCTGGACATATTGCCCCTGGCGTGAC
ACACGAACTGGCCTCTTCTCTGGATCTGCTGCCTACACTGGCTGCTTTGGCTGGCGCTCCTCT
GCCTAATGTGACCCTGGATGGCTTCGATCTGTCTCCACTGCTGCTCGGAACAGGCAAGAGCCC
TAGACAGAGCCTGTTCTTCTACCCTAGCTACCCCGATGAAGTGCGGGGAGTGTTTGCCGTGCG
GACAGGCAAGTACAAGGCCCACTTTTTTACCCAAGGCAGCGCCCACAGCGATACCACAGCTGA
TCCTGCTTGTCACGCCTCTAGCAGCCTGACAGCTCATGAACCACCTCTGCTGTACGACCTGTC
TAAGGACCCCGGCGAGAACTATAATCTGCTTGGCGGAGTTGCCGGCGCTACACCTGAAGTTCT
GCAGGCTCTGAAACAGCTCCAGCTGCTGAAAGCCCAGCTGGACGCTGCTGTGACATTTGGACC
TTCTCAGGTGGCAAGAGGCGAGGACCCTGCTCTGCAGATTTGTTGTCACCCTGGCTGTACCCC
TAGACCTGCCTGCTGTCACTGTCCTGATCCTCACGCTTGAGATTAATCAACCTCTGGATTACA
AAATTTGTGAAAGATTGACTGGTATTCTTAACTATGTTGCTCCTTTTACGCTATGTGGATACG
CTGCTTTAATGCCTTTGTATCATGCTATTGCTTCCCGTATGGCTTTCATTTTCTCCTCCTTGT
ATAAATCCTGGTTGCTGTCTCTTTATGAGGAGTTGTGGCCCGTTGTCAGGCAACGTGGCGTGG
TGTGCACTGTGTTTGCTGACGCAACCCCCACTGGTTGGGGCATTGCCACCACCTGTCAGCTCC
TTTCCGGGACTTTCGCTTTCCCCCTCCCTATTGCCACGGCGGAACTCATCGCCGCCTGCCTTG
CCCGCTGCTGGACAGGGGCTCGGCTGTTGGGCACTGACAATTCCGTGGTGTTGTCGGGGAAAT
CATCGTCCTTTCCTTGGCTGCTCGCCTGTGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTTTGGGCCGCCTCCC
CGCCTGATCCTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGGGAGGATT
GGGAAGACAATAGCAGGCATGCTCGAAATTAGGAACCCCTAGTGATGGAGTTGGCCACTCCCT
CTCTGCGCGCTCGCTCGCTCACTGAGGCCGCCCGGGCAAAGCCCGGGCGTCGGGCGACCTTTG
GTCGCCCGGCCTCAGTGAGCGAGCGAGCGCGCAGAGAGGGAGTGGCCAATCGCGACGGCCGAC
GTCTTTGTTACAACTTACTATATATATGCACACATATATATATATTTGGGTATATTGGGGGGG
TTCTAATTTAAGAAATGCATAATTGGCTATAGACAGACAGTTGTCAGAACTTGGCAATGGGTA
CGTGCAGGTTCATTATACCAAGTCTACTTGTAGTTGTTCAAAATGTATCATAATACAAGGCCG
GGCGAGGTCGTCACGCCTGTAATCCCAGCATTTTGGGAGGCTAAGGCAGGAGGATTGCTTGAG
GTCAGGAGTTTGTGACCAGCCTGGGCAACAGAGCAAGACCCTGTCTCCAAAAAGAAAAAAAAT
AATTTTTTACAAAATAAAAACAAAATGTATCATCAGACGAAATTAAATAAGAGGCAATTCATT
GTAATGACAACTTTTCCCAGCTTGACATTTAACAAAAAGTCTAAGTCCTCTTAATTCATATTT
AATGATCAAATATCAAATACTAATTTTTTTTTTTTTTTTTTTTTTGAGACGGAGTCTCGCTCT
GTCGCCCAGGCTGGAGTGCAGTGGCGCGATCCTGGCTCACTGCAAGCTCCGCCTCCCGGGTTC
ACGCCATTCTCCTGCCTCAGCCTCCCGAGTAGCTGGGATTACAGACATGCGCCACCACGCCCG
GCTAATTTTGTATTTTTAGTAGAGATGGGGTTTCTCCATGTTGGTCAGGCTGGTCTTGAATTT
CCCACCTCAGGTGATCTGCCTGCCTCAGCCTCACAAAGCAGTAGCTGGGACTACAGGCACCCA
CCACCACACTTGGTTAATTCTTTTGTATTTTTTTTGTAAAGACGGGATTTCACCATGTTAGCC
AGGATGGTCTCGATCTCCTGATCTCATGATCCGCCCGCCTCAGCCTCCCAAAGTGCTGGGATT
ACAGGCGTGAGCCACCCCGCCCGGCCATCAAATACTAATTCTTAAATGGTAAGGACCCACTAT
TCAGAACCTGTATCCTTATCACTAATATGCAAATATTTATTGAATACTTACTATGTCATGCAT
ACTAGAGAGAGTTAGATAAATTTGATACAGCTACCCTCACAGAACTTACAGTGTAATAGATGG
CATGACATGTACATGAGTAACTGTGAACAGTGTTAAATTGCTATTTAAAAAAAAAGACGGCTG
GGCGCTGTGGCTCATGCCTGTAATCCCAGCACTTTGGGAGGCCAAGGCAAGTTGATCGCTCGA
GGTCAAGAGTTCGAGACCAGCCTGGCCAACGTGGTAAAACCCCGTCTCTACTAAAAATACAAA
AAAAAAATTAGCCAGGCATGGTGGCACAGGCCTGTAATCCCAGCTACTAGGGAGGCTGAGACA
TGGAGAACTGCTTGAATCCAGGAGGCAGAGGTTACAGTGAGCCGAGATCATACCACTACACTC
CAGCCTGAGTGACAGAGCGAGACTCCTGTCTAAAAAAAAAAAAAAAAAAAAAGATACAGGTTA
AGTGTTATGGTAGTTGAAGAGAGAACTCAAACTCTGTCTCAGAAGCCTCACTTGCATGTGGAC
CACTGATATGAAATAATATAAATAGGTATAATTCAATAAATAGGAACTTCAGTTTTAATCATC
CCAAACACCAAAACTTCCTATCAAACAGGTCCAATAAACTCAATCTCTATAAGAGCTAGACAG
AAATCTACTTGGTGGCCTATAATCTTATTAGCCCTTACTTGTCCCATCTGATATTAATTAACC
CCATCTAATATGGATTAGTTAACAATCCAGTGGCTGCTTTGACAGGAACAGTTGGAGAGAGTT
GGGGATTGCAACATATTCAATTATACAAAAATGCATTCAGCATCTACCTTGATTAAGGCAGTG
TGCAACAGAATTTGCAGGAGAGTAAAAGAATGATTATAAATTTACAACCCTTAAAGAGCTATA
GCTGGGCGTGGTGGCTCATGCCTGTAAATCCCAGCACTTTGGGAGGCTGAGGCGGGTGGATCA
CCTGAGGCCAGAAGTTCAAGACCAGCCTAGCCAACATGGCGAAACCCTGTCTCTACAAAAAAT
ACAAAAATTAGCCGGGTGTGGTGGCACGTGCCTGTAGTCCCAGTTACTTGGGAGGCCGAGGCA
GGAGAATCGCTTGAACCTAGGAGGTGGAGGCTGCAGTGAGCCGAGATTGTGCCACTGCACTCC
ACTTCAGCCTGGGCGACAAGAGCAAGACTCCGTCACAAAAAAAAAAAAAAAAAAAAAGGCTAA
AATCTAGTGGGAAAGGCATATATACATACAACTAACTGTATAGCATAATAAAGCTCATAATCT
GTAACAAAATCTAATTCGACAAGCCCAGAAACTTGTGATTTACCAAAAACAGTTATATATACA
CAAAAAGTAAACCTAGAACCCAAAGTTACCCAGCACCAATGATTCTCTCCCTAAGCAGTATCA
AGTTTAAAGCAGTGATTACATTCTACTGCCTAGATTGTAAACTGAGTAAAGGAGACCAGCACC
TTTCTGCTACTGAACTAGCACAGCCGTGTAAACCAACAAGGCAATGGCAGTGCCCAACTTTCT
GTATGAATATAAGTTACATCTGTTTTATTATTTGTGACTTGGTGTTGCATGTGGTTATTATCA
ACACCTTCTGAAAGAACAACTACCTGCTCAGGCTGCCATAACAAAATACCACAGACTGAGTGA
CTTAACAGAAACTTATTTCTCACAGTTTTGGAGGCTGGGAAGTCCAAAATTAAGCTAACTGCA
AGGTAGGTTTCAATCTCAGGCCTCTTCTTTGGCTTGAAGGTCTTCTAACTGTGTGCTCACATG
ACCTCTTCTAACAAGCTCTCTGGTGTCTCTTTTTTTTTTTTTTTCTTTTTTGAGACAGAGTCT
CACTCTGTCACCCAGGCTGGAGTACAGTGGCACAATCTGGGCTCACTGCAACCTCCAACTCCC
GGGTTCAAGTGATTCTCATGCCTCACCCTCCCGAGTAGCTTGGATGACAGGAGCCCGCTACCA
CACCCAGCTAATTTTTGTATTTTTAGTAGAGATGGTGTTTCACTACATTGGCCAGGCTGGTCT
CAAACTCCTGACCTCGTGATCCACCCACCTTGGCCTCCCAAAGTGCTGGGATTACAGGTGTGA
GCCACTGCGCCCGTCCTGGTGTCTTTTCATATAAGGGCACTAATCCAATCAGACCTGGGCCCA
ACCCTCCCGACTTCTTCTAACTGTAATTACCTTCCAAAGGCCCTGTCTCCAAATACCATCACA
CTGGGGGTTAGGACTTCAAAAAAGGTATGGGGGGGGTGTGGGAGGACATAAATGCTCAGTCCA
TAACAAGCACCCAACATAAAAATGGCTAGAACAGATCACAAAAAAAAGGTCCTGTATGGCTTT
GGGGAAGGGCTCAACCCCAAAATATCTGAAAGCTCTGGAGGGGCCTAGAAGTGGTAAATGAAT
GAAAACGTGGTTACTCTCCCGATCTGCCTTTCCCAAATATGGCCATTCTTGGCTGAATCAGAA
ATCAAAGGACAGGTTATTAATTACTAGCTCTAAGTTACTTACCATTTGCTGAGACAGTTCAGA
AATCTGACTGCATCTCCTCAGGGATCTAGAACACAGTTCTCAAATTCTAACTTACTTGTGATA
TACTTGTGAATGATAAAAATCGCTACAGGTACTTTTATTAATCTGAAAGAGTATTGAGAAATT
ACCTTTCATTCTGACTTTTGTCTGGAATGAAAATCAATACTTTTGCTATATTCCATTACTGAA
ATAATTTTACTTTCCAGTAAAACTGGCATTATAATTTTTTTTAATTTTTAAAACTTCATAATT
TTTTGCCAGACTGACCCATGTAAACATACAAATTACTAATAATTATGCACGTCACATCTGTAA
TAATGGCCTTCATGTAAACATTTTTGTGGTTTACACATAAAATCTCTAATTACAAAGCTATAT
TATCTAAAATTACAGTAAGCAAGAAAATTAATCCAAGCTAAGACAATACTTGCAACATCAATT
CATCATCTGTGACAAGGACTGCTTAAGTCTCTTTGTGGTTGACGTCATTAATTAACTGGCCTC
ATGGGCCTTCCGCTCACTGCCCGCTTTCCAGTCGGGAAACCTGTCGTGCCAGCTGCATTAACA
TGGTCATAGCTGTTTCCTTGCGTATTGGGCGCTCTCCGCTTCCTCGCTCACTGACTCGCTGCG
CTCGGTCGTTCGGGTAAAGCCTGGGGTGCCTAATGAGCAAAAGGCCAGCAAAAGGCCAGGAAC
CGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGG
TGGCCTAACTACGGCTACACTAGAAGAACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCA
GCGATCTGTCTATTTCGTTCATCCATAGTTGCCTGACTCCCCGTCGTGTAGATAACTACGATA
CGGGAGGGCTTACCATCTGGCCCCAGTGCTGCAATGATACCGCGAGAACCACGCTCACCGGCT
CCAGATTTATCAGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTT
TCTGTGACTGGTGAGTACTCAACCAAGTCATTCTGAGAATAGTGTATGCGGCGACCGAGTTGC
TCTTGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCCCGA
AAAGTGCCACCTAAATTGTAAGCGTTAATATTTTGTTAAAATTCGCGTTAAATTTTTGTTAAA
TCAGCTCATTTTTTAACCAATAGGCCGAAATCGGCAAAATCCCTTATAAATCAAAAGAATAGA
CCGAGATAGGGTTGAGTGGCCGCTACAGGGCGCTCCCATTCGCCATTCAGGCTGCGCAACTGT
TGGGAAGGGCGTTTCGGTGCGGGCCTCTTCGCTATTACGCCAGCTGGCGAAAGGGGGATGTGC
TGCAAGGCGATTAAGTTGGGTAACGCCAGGGTTTTCCCAGTCACGACGTTGTAAAACGACGGC
CAGTGAGCGCGACGTAATACGACTCACTATAGGGCGAATTGGCGGAAGGCCGTCAAGGCCTAG
GCGCGCCAACGCGT
Underlined, codon Optimized ARSA DNA sequence (Human)
5′ AAV2 ITR DNA Sequence
(SEQ ID NO: 4)
TTGGCCACTCCCTCTCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGCCAAC
TCCATCACTAGGGGTTCCT
3′ AAV2 ITR DNA Sequence in Flip Orientation
(SEQ ID NO: 5)
AGGAACCCCTAGTGATGGAGTTGGCCACTCCCTCTCTGCGCGCTCGCTCGCTCACTGAGGCCG
CCCGGGCAAAGCCCGGGCGTCGGGCGACCTTTGGTCGCCCGGCCTCAGTGAGCGAGCGAGCGC
GCAGAGAGGGAGTGGCCAA
CMV Enhancer Element DNA Sequence (GenBank: K03104.1)
(SEQ ID NO: 6)
GACATTGATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCA
TGG
Chicken-Actin Promoter DNA Sequence
(SEQ ID NO: 7)
TCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCG
CGCCAGGCGGGGCGGGGCGGGGCGAGGGGGGGGGGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTA
TAAAAAGCGAAGCGCGCGGCGGGCGGGAGTCGCTGCGCGCTGCCTTCGCCCCGTGCCCCGCTC
CGCCGCC
WPRE Element DNA Sequence
(SEQ ID NO: 8)
TTAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATGG
CTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTGTGTTGCCACCTGGA
TTCTGCGCGGGACGTCCTTCTGCTACGTCCCTTCGGCCCTCAATCCAGCGGACCTTCCTTCCC
GCGGCCTGCTGCCGGCTCTGCGGCCTCTTCCGCGTCTTCGCCTTCGCCCTCAGACGAGTCGGA
TCTCCCTTTGGGCCGCCTCCCCGCCTG
(SEQ ID NO: 9)
Met Ala Ala Asp Gly Tyr Leu Pro Asp Trp Leu Glu Asp Asn Leu Ser
1 5 10 15
Glu Gly Ile Arg Glu Trp Trp Ala Leu Lys Pro Gly Ala Pro Gln Pro
                        20 25 30
Lys Ala Asn Gln Gln His Gln Asp Asn Ala Arg Gly Leu Val Leu Pro
                35 40 45
Gly Tyr Lys Tyr Leu Gly Pro Gly Asn Gly Leu Asp Lys Gly Glu Pro
          50 55 60
Val Asn Ala Ala Asp Ala Ala Ala Leu Glu His Asp Lys Ala Tyr Asp
65 70 75 80
Gln Gln Leu Lys Ala Gly Asp Asn Pro Tyr Leu Lys Tyr Asn His Ala
                                85 90 95
Asp Ala Glu Phe Gln Glu Arg Leu Lys Glu Asp Thr Ser Phe Gly Gly
                        100 105 110
Asn Leu Gly Arg Ala Val Phe Gln Ala Lys Lys Arg Leu Leu Glu Pro
                115 120 125
Leu Gly Leu Val Glu Glu Ala Ala Lys Thr Ala Pro Gly Lys Lys Arg
      130 135 140
Pro Val Glu Gln Ser Pro Gln Glu Pro Asp Ser Ser Ala Gly Ile Gly
145 150 155 160
Lys Ser Gly Ala Gln Pro Ala Lys Lys Arg Leu Asn Phe Gly Gln Thr
                                165 170 175
Gly Asp Thr Glu Ser Val Pro Asp Pro Gln Pro Ile Gly Glu Pro Pro
                        180 185 190
Ala Ala Pro Ser Gly Val Gly Ser Leu Thr Met Ala Ser Gly Gly Gly
                195 200 205
Ala Pro Val Ala Asp Asn Asn Glu Gly Ala Asp Gly Val Gly Ser Ser
        210 215 220
Ser Gly Asn Trp His Cys Asp Ser Gln Trp Leu Gly Asp Arg Val Ile
225 230 235 240
Thr Thr Ser Thr Arg Thr Trp Ala Leu Pro Thr Tyr Asn Asn His Leu
                                245 250 255
Tyr Lys Gln Ile Ser Asn Ser Thr Ser Gly Gly Ser Ser Asn Asp Asn
                        260 265 270
Ala Tyr Phe Gly Tyr Ser Thr Pro Trp Gly Tyr Phe Asp Phe Asn Arg
                275 280 285
Phe His Cys His Phe Ser Pro Arg Asp Trp Gln Arg Leu Ile Asn Asn
        290 295 300
Asn Trp Gly Phe Arg Pro Lys Arg Leu Asn Phe Lys Leu Phe Asn Ile
305 310 315 320
Gln Val Lys Glu Val Thr Asp Asn Asn Gly Val Lys Thr Ile Ala Asn
                                325 330 335
Asn Leu Thr Ser Thr Val Gln Val Phe Thr Asp Ser Asp Tyr Gln Leu
                        340 345 350
Pro Tyr Val Leu Gly Ser Ala His Glu Gly Cys Leu Pro Pro Phe Pro
                355 360 365
Ala Asp Val Phe Met Ile Pro Gln Tyr Gly Tyr Leu Thr Leu Asn Asp
        370 375 380
Gly Ser Gln Ala Val Gly Arg Ser Ser Phe Tyr Cys Leu Glu Tyr Phe
385 390 395 400
Pro Ser Gln Met Leu Arg Thr Gly Asn Asn Phe Gln Phe Ser Tyr Glu
                              405 410 415
Phe Glu Asn Val Pro Phe His Ser Ser Tyr Ala His Ser Gln Ser Leu
                        420 425 430
Asp Arg Leu Met Asn Pro Leu Ile Asp Gln Tyr Leu Tyr Tyr Leu Ser
                435 440 445
Lys Thr Ile Asn Gly Ser Gly Gln Asn Gln Gln Thr Leu Lys Phe Ser
        450 455 460
Val Ala Gly Pro Ser Asn Met Ala Val Gln Gly Arg Asn Tyr Ile Pro
465 470 475 480
Gly Pro Ser Tyr Arg Gln Gln Arg Val Ser Thr Thr Val Thr Gln Asn
                                485 490 495
Asn Asn Ser Glu Phe Ala Trp Pro Gly Ala Ser Ser Trp Ala Leu Asn
                        500 505 510
Gly Arg Asn Ser Leu Met Asn Pro Gly Pro Ala Met Ala Ser His Lys
                515 520 525
Glu Gly Glu Asp Arg Phe Phe Pro Leu Ser Gly Ser Leu Ile Phe Gly
        530 535 540
Lys Gln Gly Thr Gly Arg Asp Asn Val Asp Ala Asp Lys Val Met Ile
545 550 555 560
Thr Asn Glu Glu Glu Ile Lys Thr Thr Asn Pro Val Ala Thr Glu Ser
                                565 570 575
Tyr Gly Gln Val Ala Thr Asn His Gln Ser Ala Gln Ala Gln Ala Gln
                        580 585 590
Thr Gly Trp Val Gln Asn Gln Gly Ile Leu Pro Gly Met Val Trp Gln
                595 600 605
Asp Arg Asp Val Tyr Leu Gln Gly Pro Ile Trp Ala Lys Ile Pro His
        610 615 620
Thr Asp Gly Asn Phe His Pro Ser Pro Leu Met Gly Gly Phe Gly Met
625 630 635 640
Lys His Pro Pro Pro Gln Ile Leu Ile Lys Asn Thr Pro Val Pro Ala
                                645 650 655
Asp Pro Pro Thr Ala Phe Asn Lys Asp Lys Leu Asn Ser Phe Ile Thr
                        660 665 670
Gln Tyr Ser Thr Gly Gln Val Ser Val Glu Ile Glu Trp Glu Leu Gln
                675 680 685
Lys Glu Asn Ser Lys Arg Trp Asn Pro Glu Ile Gln Tyr Thr Ser Asn
        690 695 700
Tyr Tyr Lys Ser Asn Asn Val Glu Phe Ala Val Asn Thr Glu Gly Val
705 710 715 720
Tyr Ser Glu Pro Arg Pro Ile Gly Thr Arg Tyr Leu Thr Arg Asn Leu
                              725 730 735
Targeting Peptide Amino Acid Sequence
(SEQ ID NO: 10)
KGGGFHG
Targeting Peptide Flanked by Linkers - Amino Acid Sequence
(SEQ ID NO: 11)
AAAKGGGFHGAS
SAN0006 Capsid Amino Acid Sequence (Full Structural Protein)
(SEQ ID NO: 12)
Met Ala Ala Asp Gly Tyr Leu Pro Asp Trp Leu Glu Asp Asn Leu Ser
1 5 10 15
Glu Gly Ile Arg Glu Trp Trp Ala Leu Lys Pro Gly Ala Pro Gln Pro
                        20 25 30
Lys Ala Asn Gln Gln His Gln Asp Asn Ala Arg Gly Leu Val Leu Pro
                35 40 45
Gly Tyr Lys Tyr Leu Gly Pro Gly Asn Gly Leu Asp Lys Gly Glu Pro
          50 55 60
Val Asn Ala Ala Asp Ala Ala Ala Leu Glu His Asp Lys Ala Tyr Asp
65 70 75 80
Gln Gln Leu Lys Ala Gly Asp Asn Pro Tyr Leu Lys Tyr Asn His Ala
                                85 90 95
Asp Ala Glu Phe Gln Glu Arg Leu Lys Glu Asp Thr Ser Phe Gly Gly
                        100 105 110
Asn Leu Gly Arg Ala Val Phe Gln Ala Lys Lys Arg Leu Leu Glu Pro
                115 120 125
Leu Gly Leu Val Glu Glu Ala Ala Lys Thr Ala Pro Gly Lys Lys Arg
      130 135 140
Pro Val Glu Gln Ser Pro Gln Glu Pro Asp Ser Ser Ala Gly Ile Gly
145 150 155 160
Lys Ser Gly Ala Gln Pro Ala Lys Lys Arg Leu Asn Phe Gly Gln Thr
                                165 170 175
Gly Asp Thr Glu Ser Val Pro Asp Pro Gln Pro Ile Gly Glu Pro Pro
                        180 185 190
Ala Ala Pro Ser Gly Val Gly Ser Leu Thr Met Ala Ser Gly Gly Gly
                195 200 205
Ala Pro Val Ala Asp Asn Asn Glu Gly Ala Asp Gly Val Gly Ser Ser
        210 215 220
Ser Gly Asn Trp His Cys Asp Ser Gln Trp Leu Gly Asp Arg Val Ile
225 230 235 240
Thr Thr Ser Thr Arg Thr Trp Ala Leu Pro Thr Tyr Asn Asn His Leu
                                245 250 255
Tyr Lys Gln Ile Ser Asn Ser Thr Ser Gly Gly Ser Ser Asn Asp Asn
                        260 265 270
Ala Tyr Phe Gly Tyr Ser Thr Pro Trp Gly Tyr Phe Asp Phe Asn Arg
                275 280 285
Phe His Cys His Phe Ser Pro Arg Asp Trp Gln Arg Leu Ile Asn Asn
        290 295 300
Asn Trp Gly Phe Arg Pro Lys Arg Leu Asn Phe Lys Leu Phe Asn Ile
305 310 315 320
Gln Val Lys Glu Val Thr Asp Asn Asn Gly Val Lys Thr Ile Ala Asn
                                325 330 335
Asn Leu Thr Ser Thr Val Gln Val Phe Thr Asp Ser Asp Tyr Gln Leu
                        340 345 350
Pro Tyr Val Leu Gly Ser Ala His Glu Gly Cys Leu Pro Pro Phe Pro
                355 360 365
Ala Asp Val Phe Met Ile Pro Gln Tyr Gly Tyr Leu Thr Leu Asn Asp
        370 375 380
Gly Ser Gln Ala Val Gly Arg Ser Ser Phe Tyr Cys Leu Glu Tyr Phe
385 390 395 400
Pro Ser Gln Met Leu Arg Thr Gly Asn Asn Phe Gln Phe Ser Tyr Glu
                              405 410 415
Phe Glu Asn Val Pro Phe His Ser Ser Tyr Ala His Ser Gln Ser Leu
                        420 425 430
Asp Arg Leu Met Asn Pro Leu Ile Asp Gln Tyr Leu Tyr Tyr Leu Ser
                435 440 445
Lys Thr Ile Asn Gly Ser Gly Gln Asn Gln Gln Thr Leu Lys Phe Ser
        450 455 460
Val Ala Gly Pro Ser Asn Met Ala Val Gln Gly Arg Asn Tyr Ile Pro
465 470 475 480
Gly Pro Ser Tyr Arg Gln Gln Arg Val Ser Thr Thr Val Thr Gln Asn
                                485 490 495
Asn Asn Ser Glu Phe Ala Trp Pro Gly Ala Ser Ser Trp Ala Leu Asn
                        500 505 510
Gly Arg Asn Ser Leu Met Asn Pro Gly Pro Ala Met Ala Ser His Lys
                515 520 525
Glu Gly Glu Asp Arg Phe Phe Pro Leu Ser Gly Ser Leu Ile Phe Gly
        530 535 540
Lys Gln Gly Thr Gly Arg Asp Asn Val Asp Ala Asp Lys Val Met Ile
545 550 555 560
Thr Asn Glu Glu Glu Ile Lys Thr Thr Asn Pro Val Ala Thr Glu Ser
                                565 570 575
Tyr Gly Gln Val Ala Thr Asn His Gln Ser Ala Gln Ala Ala Ala Lys
                        580 585 590
Gly Gly Gly Phe His Gly Ala Ser Ala Gln Ala Gln Thr Gly Trp Val
                595 600 605
Gln Asn Gln Gly Ile Leu Pro Gly Met Val Trp Gln Asp Arg Asp Val
        610 615 620
Tyr Leu Gln Gly Pro Ile Trp Ala Lys Ile Pro His Thr Asp Gly Asn
625 630 635 640
Phe His Pro Ser Pro Leu Met Gly Gly Phe Gly Met Lys His Pro Pro
                                645 650 655
Pro Gln Ile Leu Ile Lys Asn Thr Pro Val Pro Ala Asp Pro Pro Thr
                        660 665 670
Ala Phe Asn Lys Asp Lys Leu Asn Ser Phe Ile Thr Gln Tyr Ser Thr
                675 680 685
Gly Gln Val Ser Val Glu Ile Glu Trp Glu Leu Gln Lys Glu Asn Ser
        690 695 700
Lys Arg Trp Asn Pro Glu Ile Gln Tyr Thr Ser Asn Tyr Tyr Lys Ser
705 710 715 720
Asn Asn Val Glu Phe Ala Val Asn Thr Glu Gly Val Tyr Ser Glu Pro
                              725 730 735
Arg Pro Ile Gly Thr Arg Tyr Leu Thr Arg Asn Leu
                        740 745
AAV9 VP2 Capsid Amino Acid Sequence
(SEQ ID NO: 13)
Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Ala Ala Lys
Gly Gly Gly Phe His Gly Ala Ser Ala Gln Ala Gln Thr Gly Trp Val
Gln Asn Gln Gly Ile Leu Pro Gly Met Val Trp Gln Asp Arg Asp Val
Tyr Leu Gln Gly Pro Ile Trp Ala Lys Ile Pro His Thr Asp Gly Asn
Phe His Pro Ser Pro Leu Met Gly Gly Phe Gly Met Lys His Pro Pro
Pro Gln Ile Leu Ile Lys Asn Thr Pro Val Pro Ala Asp Pro Pro Thr
Ala Phe Asn Lys Asp Lys Leu Asn Ser Phe Ile Thr Gln Tyr Ser Thr
Gly Gln Val Ser Val Glu Ile Glu Trp Glu Leu Gln Lys Glu Asn Ser
Lys Arg Trp Asn Pro Glu Ile Gln Tyr Thr Ser Asn Tyr Tyr Lys Ser
Asn Asn Val Glu Phe Ala Val Asn Thr Glu Gly Val Tyr Ser Glu Pro
Arg Pro Ile Gly Thr Arg Tyr Leu Thr Arg Asn Leu
AAV9 VP3 Capsid Amino Acid Sequence
(SEQ ID NO: 14)
Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Gln Ala Gln
Thr Gly Trp Val Gln Asn Gln Gly Ile Leu Pro Gly Met Val Trp Gln
Asp Arg Asp Val Tyr Leu Gln Gly Pro Ile Trp Ala Lys Ile Pro His
Thr Asp Gly Asn Phe His Pro Ser Pro Leu Met Gly Gly Phe Gly Met
Lys His Pro Pro Pro Gln Ile Leu Ile Lys Asn Thr Pro Val Pro Ala
Asp Pro Pro Thr Ala Phe Asn Lys Asp Lys Leu Asn Ser Phe Ile Thr
Gln Tyr Ser Thr Gly Gln Val Ser Val Glu Ile Glu Trp Glu Leu Gln
Lys Glu Asn Ser Lys Arg Trp Asn Pro Glu Ile Gln Tyr Thr Ser Asn
Tyr Tyr Lys Ser Asn Asn Val Glu Phe Ala Val Asn Thr Glu Gly Val
Tyr Ser Glu Pro Arg Pro Ile Gly Thr Arg Tyr Leu Thr Arg Asn Leu
GCase Polypeptide Sequence (with wild-type signaling peptide)
(SEQ ID NO: 17)
MEFSSPSREECPKPLSRVSIMAGSLTGLLLLQAVSWASGARPCIPKSFGYSSVVCVCNATYCD
SFDPPTFPALGTFSRYESTRSGRRMELSMGPIQANHTGTGLLLTLQPEQKFQKVKGFGGAMTD
AAALNILALSPPAQNLLLKSYFSEEGIGYNIIRVPMASCDFSIRTYTYADTPDDFQLHNFSLP
EEDTKLKIPLIHRALQLAQRPVSLLASPWTSPTWLKTNGAVNGKGSLKGQPGDIYHQTWARYF
VKFLDAYAEHKLQFWAVTAENEPSAGLLSGYPFQCLGFTPEHQRDFIARDLGPTLANSTHHNV
RLLMLDDQRLLLPHWAKVVLTDPEAAKYVHGIAVHWYLDFLAPAKATLGETHRLFPNTMLFAS
EACVGSKFWEQSVRLGSWDRGMQYSHSIITNLLYHVVGWTDWNLALNPEGGPNWVRNFVDSPI
IVDITKDTFYKQPMFYHLGHFSKFIPEGSQRVGLVASQKNDLDAVALMHPDGSAVVVVLNRSS
KDVPLTIKDPAVGFLETISPGYSIHTYLWRRQ
GCase Polypeptide Sequence (following cleavage of signaling peptide)
(SEQ ID NO: 18)
ARPCIPKSFGYSSVVCVCNATYCDSFDPPTFPALGTFSRYESTRSGRRMELSMGPIQANHTGT
GLLLTLQPEQKFQKVKGFGGAMTDAAALNILALSPPAQNLLLKSYFSEEGIGYNIIRVPMASC
DFSIRTYTYADTPDDFQLHNFSLPEEDTKLKIPLIHRALQLAQRPVSLLASPWTSPTWLKTNG
AVNGKGSLKGQPGDIYHQTWARYFVKFLDAYAEHKLQFWAVTAENEPSAGLLSGYPFQCLGFT
PEHQRDFIARDLGPTLANSTHHNVRLLMLDDQRLLLPHWAKVVLTDPEAAKYVHGIAVHWYLD
FLAPAKATLGETHRLFPNTMLFASEACVGSKFWEQSVRLGSWDRGMQYSHSIITNLLYHVVGW
TDWNLALNPEGGPNWVRNFVDSPIIVDITKDTFYKQPMFYHLGHFSKFIPEGSQRVGLVASQK
NDLDAVALMHPDGSAVVVVLNRSSKDVPLTIKDPAVGFLETISPGYSIHTYLWRRQ
Codon-Optimized Human GBA1 Sequence with Endogenous Signaling
Peptide
(SEQ ID NO: 19)
ATGGAGTTCAGCTCTCCGAGCCGGGAAGAGTGCCCCAAACCCCTGTCGAGAGTGTCCATCATG
GCCGGCAGCCTGACCGGCCTGCTGCTGCTGCAAGCTGTTAGCTGGGCCAGCGGGGCCAGACCT
TGTATCCCCAAGAGCTTCGGATATAGCAGCGTGGTCTGCGTGTGCAACGCCACCTACTGCGAT
AGCTTCGACCCACCTACCTTTCCAGCTCTGGGCACCTTCTCCAGATACGAGTCTACAAGAAGC
GGCAGAAGAATGGAACTGTCCATGGGCCCTATCCAGGCCAACCACACCGGCACAGGCCTCCTG
TTGACCCTGCAGCCCGAGCAGAAATTTCAGAAAGTGAAGGGATTCGGCGGCGCCATGACCGAT
GCCGCCGCTCTGAATATCCTGGCGCTGAGCCCTCCTGCCCAGAACCTGCTGCTGAAGAGCTAC
TTTAGCGAGGAGGGGATCGGCTACAACATTATCAGAGTGCCCATGGCCAGCTGCGACTTTAGC
ATCAGAACCTATACATACGCCGACACCCCAGATGACTTCCAGCTGCACAACTTCAGCCTGCCT
GAGGAAGATACAAAGCTGAAAATCCCCCTGATCCACCGGGCCCTGCAACTGGCTCAGCGGCCA
GTGTCCCTGCTGGCCTCTCCTTGGACCAGCCCTACCTGGCTGAAGACCAATGGCGCCGTGAAC
GGCAAGGGCTCTCTGAAGGGCCAGCCTGGCGACATCTACCATCAGACCTGGGCCAGATACTTC
GTGAAGTTCCTGGATGCCTACGCTGAGCACAAGCTGCAGTTCTGGGCCGTGACCGCCGAGAAC
GAGCCTTCCGCCGGACTGCTCAGCGGCTATCCTTTCCAGTGCCTGGGCTTCACACCTGAGCAC
CAGCGCGACTTCATCGCCAGGGACCTGGGCCCTACACTGGCCAATAGCACACACCACAACGTG
AGACTGCTGATGCTGGATGATCAGAGACTGCTGCTTCCCCACTGGGCTAAGGTCGTGCTGACC
GACCCTGAAGCCGCTAAGTACGTGCACGGCATCGCCGTGCACTGGTACCTGGACTTCCTGGCC
CCTGCCAAGGCCACCCTGGGAGAAACCCACCGGCTGTTCCCTAACACCATGCTGTTTGCCTCA
GAGGCCTGTGTGGGCTCCAAATTCTGGGAGCAAAGCGTGCGGCTGGGCTCTTGGGACAGAGGA
ATGCAGTACAGCCACAGCATCATCACCAACCTGCTGTACCACGTGGTGGGCTGGACCGACTGG
AACCTGGCCCTGAACCCCGAGGGAGGCCCTAACTGGGTCAGAAACTTCGTGGACAGCCCCATC
ATTGTGGATATCACCAAGGACACGTTCTACAAGCAGCCAATGTTCTACCACCTGGGCCACTTC
AGCAAGTTCATCCCTGAAGGCAGTCAGAGAGTGGGCCTGGTGGCCTCTCAGAAGAACGACCTG
GACGCCGTTGCGCTGATGCACCCCGACGGCAGCGCCGTGGTGGTGGTGCTGAATAGATCTTCT
AAGGATGTGCCTCTGACAATCAAGGACCCTGCTGTGGGATTTCTGGAAACAATCAGCCCTGGA
TACAGCATCCATACATACCTGTGGCGGCGGCAGTGA
Codon-Optimized Human GBA1 Sequence with Engineered Signaling
Peptide (SS1)
(SEQ ID NO: 20)
ATGGCCTCTCTGTGGCTGCTGAGCTGCTTCAGCCTGGTGGGCGCCGCTTTCGGCGCCAGACCT
TGTATCCCCAAGAGCTTCGGATATAGCAGCGTGGTCTGCGTGTGCAACGCCACCTACTGCGAT
AGCTTCGACCCACCTACCTTTCCAGCTCTGGGCACCTTCTCCAGATACGAGTCTACAAGAAGC
GGCAGAAGAATGGAACTGTCCATGGGCCCTATCCAGGCCAACCACACCGGCACAGGCCTCCTG
TTGACCCTGCAGCCCGAGCAGAAATTTCAGAAAGTGAAGGGATTCGGCGGCGCCATGACCGAT
GCCGCCGCTCTGAATATCCTGGCGCTGAGCCCTCCTGCCCAGAACCTGCTGCTGAAGAGCTAC
TTTAGCGAGGAGGGGATCGGCTACAACATTATCAGAGTGCCCATGGCCAGCTGCGACTTTAGC
ATCAGAACCTATACATACGCCGACACCCCAGATGACTTCCAGCTGCACAACTTCAGCCTGCCT
GAGGAAGATACAAAGCTGAAAATCCCCCTGATCCACCGGGCCCTGCAACTGGCTCAGCGGCCA
GTGTCCCTGCTGGCCTCTCCTTGGACCAGCCCTACCTGGCTGAAGACCAATGGCGCCGTGAAC
GGCAAGGGCTCTCTGAAGGGCCAGCCTGGCGACATCTACCATCAGACCTGGGCCAGATACTTC
GTGAAGTTCCTGGATGCCTACGCTGAGCACAAGCTGCAGTTCTGGGCCGTGACCGCCGAGAAC
GAGCCTTCCGCCGGACTGCTCAGCGGCTATCCTTTCCAGTGCCTGGGCTTCACACCTGAGCAC
CAGCGCGACTTCATCGCCAGGGACCTGGGCCCTACACTGGCCAATAGCACACACCACAACGTG
AGACTGCTGATGCTGGATGATCAGAGACTGCTGCTTCCCCACTGGGCTAAGGTCGTGCTGACC
GACCCTGAAGCCGCTAAGTACGTGCACGGCATCGCCGTGCACTGGTACCTGGACTTCCTGGCC
CCTGCCAAGGCCACCCTGGGAGAAACCCACCGGCTGTTCCCTAACACCATGCTGTTTGCCTCA
GAGGCCTGTGTGGGCTCCAAATTCTGGGAGCAAAGCGTGCGGCTGGGCTCTTGGGACAGAGGA
ATGCAGTACAGCCACAGCATCATCACCAACCTGCTGTACCACGTGGTGGGCTGGACCGACTGG
AACCTGGCCCTGAACCCCGAGGGAGGCCCTAACTGGGTCAGAAACTTCGTGGACAGCCCCATC
ATTGTGGATATCACCAAGGACACGTTCTACAAGCAGCCAATGTTCTACCACCTGGGCCACTTC
AGCAAGTTCATCCCTGAAGGCAGTCAGAGAGTGGGCCTGGTGGCCTCTCAGAAGAACGACCTG
GACGCCGTTGCGCTGATGCACCCCGACGGCAGCGCCGTGGTGGTGGTGCTGAATAGATCTTCT
AAGGATGTGCCTCTGACAATCAAGGACCCTGCTGTGGGATTTCTGGAAACAATCAGCCCTGGA
TACAGCATCCATACATACCTGTGGCGGCGGCAGTGA
Codon-Optimized Human GBA1 Sequence with Engineered Signaling
Peptide (SS2)
(SEQ ID NO: 21)
ATGTTCCAGACCGGCGGACTGATCGTGTTCTACGGCCTGCTGGCCCAGACAATGGCCGCCAGA
CCTTGTATCCCCAAGAGCTTCGGATATAGCAGCGTGGTCTGCGTGTGCAACGCCACCTACTGC
GATAGCTTCGACCCACCTACCTTTCCAGCTCTGGGCACCTTCTCCAGATACGAGTCTACAAGA
AGCGGCAGAAGAATGGAACTGTCCATGGGCCCTATCCAGGCCAACCACACCGGCACAGGCCTC
CTGTTGACCCTGCAGCCCGAGCAGAAATTTCAGAAAGTGAAGGGATTCGGCGGCGCCATGACC
GATGCCGCCGCTCTGAATATCCTGGCGCTGAGCCCTCCTGCCCAGAACCTGCTGCTGAAGAGC
TACTTTAGCGAGGAGGGGATCGGCTACAACATTATCAGAGTGCCCATGGCCAGCTGCGACTTT
AGCATCAGAACCTATACATACGCCGACACCCCAGATGACTTCCAGCTGCACAACTTCAGCCTG
CCTGAGGAAGATACAAAGCTGAAAATCCCCCTGATCCACCGGGCCCTGCAACTGGCTCAGCGG
CCAGTGTCCCTGCTGGCCTCTCCTTGGACCAGCCCTACCTGGCTGAAGACCAATGGCGCCGTG
AACGGCAAGGGCTCTCTGAAGGGCCAGCCTGGCGACATCTACCATCAGACCTGGGCCAGATAC
TTCGTGAAGTTCCTGGATGCCTACGCTGAGCACAAGCTGCAGTTCTGGGCCGTGACCGCCGAG
AACGAGCCTTCCGCCGGACTGCTCAGCGGCTATCCTTTCCAGTGCCTGGGCTTCACACCTGAG
CACCAGCGCGACTTCATCGCCAGGGACCTGGGCCCTACACTGGCCAATAGCACACACCACAAC
GTGAGACTGCTGATGCTGGATGATCAGAGACTGCTGCTTCCCCACTGGGCTAAGGTCGTGCTG
ACCGACCCTGAAGCCGCTAAGTACGTGCACGGCATCGCCGTGCACTGGTACCTGGACTTCCTG
GCCCCTGCCAAGGCCACCCTGGGAGAAACCCACCGGCTGTTCCCTAACACCATGCTGTTTGCC
TCAGAGGCCTGTGTGGGCTCCAAATTCTGGGAGCAAAGCGTGCGGCTGGGCTCTTGGGACAGA
GGAATGCAGTACAGCCACAGCATCATCACCAACCTGCTGTACCACGTGGTGGGCTGGACCGAC
TGGAACCTGGCCCTGAACCCCGAGGGAGGCCCTAACTGGGTCAGAAACTTCGTGGACAGCCCC
ATCATTGTGGATATCACCAAGGACACGTTCTACAAGCAGCCAATGTTCTACCACCTGGGCCAC
TTCAGCAAGTTCATCCCTGAAGGCAGTCAGAGAGTGGGCCTGGTGGCCTCTCAGAAGAACGAC
CTGGACGCCGTTGCGCTGATGCACCCCGACGGCAGCGCCGTGGTGGTGGTGCTGAATAGATCT
TCTAAGGATGTGCCTCTGACAATCAAGGACCCTGCTGTGGGATTTCTGGAAACAATCAGCCCT
GGATACAGCATCCATACATACCTGTGGCGGCGGCAGTGA
Codon-Optimized Human GBA1 Sequence with Engineered Signaling
Peptide (SS3)
(SEQ ID NO: 22)
ATGCTGCTGCTGCTGCTGCTGCTCGGCCTGAGACTGCAGCTGAGCCTGGGCGCCAGACCTTGT
ATCCCCAAGAGCTTCGGATATAGCAGCGTGGTCTGCGTGTGCAACGCCACCTACTGCGATAGC
TTCGACCCACCTACCTTTCCAGCTCTGGGCACCTTCTCCAGATACGAGTCTACAAGAAGCGGC
AGAAGAATGGAACTGTCCATGGGCCCTATCCAGGCCAACCACACCGGCACAGGCCTCCTGTTG
ACCCTGCAGCCCGAGCAGAAATTTCAGAAAGTGAAGGGATTCGGCGGCGCCATGACCGATGCC
GCCGCTCTGAATATCCTGGCGCTGAGCCCTCCTGCCCAGAACCTGCTGCTGAAGAGCTACTTT
AGCGAGGAGGGGATCGGCTACAACATTATCAGAGTGCCCATGGCCAGCTGCGACTTTAGCATC
AGAACCTATACATACGCCGACACCCCAGATGACTTCCAGCTGCACAACTTCAGCCTGCCTGAG
GAAGATACAAAGCTGAAAATCCCCCTGATCCACCGGGCCCTGCAACTGGCTCAGCGGCCAGTG
TCCCTGCTGGCCTCTCCTTGGACCAGCCCTACCTGGCTGAAGACCAATGGCGCCGTGAACGGC
AAGGGCTCTCTGAAGGGCCAGCCTGGCGACATCTACCATCAGACCTGGGCCAGATACTTCGTG
AAGTTCCTGGATGCCTACGCTGAGCACAAGCTGCAGTTCTGGGCCGTGACCGCCGAGAACGAG
CCTTCCGCCGGACTGCTCAGCGGCTATCCTTTCCAGTGCCTGGGCTTCACACCTGAGCACCAG
CGCGACTTCATCGCCAGGGACCTGGGCCCTACACTGGCCAATAGCACACACCACAACGTGAGA
CTGCTGATGCTGGATGATCAGAGACTGCTGCTTCCCCACTGGGCTAAGGTCGTGCTGACCGAC
CCTGAAGCCGCTAAGTACGTGCACGGCATCGCCGTGCACTGGTACCTGGACTTCCTGGCCCCT
GCCAAGGCCACCCTGGGAGAAACCCACCGGCTGTTCCCTAACACCATGCTGTTTGCCTCAGAG
GCCTGTGTGGGCTCCAAATTCTGGGAGCAAAGCGTGCGGCTGGGCTCTTGGGACAGAGGAATG
CAGTACAGCCACAGCATCATCACCAACCTGCTGTACCACGTGGTGGGCTGGACCGACTGGAAC
CTGGCCCTGAACCCCGAGGGAGGCCCTAACTGGGTCAGAAACTTCGTGGACAGCCCCATCATT
GTGGATATCACCAAGGACACGTTCTACAAGCAGCCAATGTTCTACCACCTGGGCCACTTCAGC
AAGTTCATCCCTGAAGGCAGTCAGAGAGTGGGCCTGGTGGCCTCTCAGAAGAACGACCTGGAC
GCCGTTGCGCTGATGCACCCCGACGGCAGCGCCGTGGTGGTGGTGCTGAATAGATCTTCTAAG
GATGTGCCTCTGACAATCAAGGACCCTGCTGTGGGATTTCTGGAAACAATCAGCCCTGGATAC
AGCATCCATACATACCTGTGGCGGCGGCAGTGA
Codon-Optimized Human GBA1 Sequence with Engineered Signaling
Peptide (SS4)
(SEQ ID NO: 23)
ATGACCATCCTGTTCCTGACAATGGTGATCAGCTACTTCGGCTGCATGAAGGCCGCCAGACCT
TGTATCCCCAAGAGCTTCGGATATAGCAGCGTGGTCTGCGTGTGCAACGCCACCTACTGCGAT
AGCTTCGACCCACCTACCTTTCCAGCTCTGGGCACCTTCTCCAGATACGAGTCTACAAGAAGC
GGCAGAAGAATGGAACTGTCCATGGGCCCTATCCAGGCCAACCACACCGGCACAGGCCTCCTG
TTGACCCTGCAGCCCGAGCAGAAATTTCAGAAAGTGAAGGGATTCGGCGGCGCCATGACCGAT
GCCGCCGCTCTGAATATCCTGGCGCTGAGCCCTCCTGCCCAGAACCTGCTGCTGAAGAGCTAC
TTTAGCGAGGAGGGGATCGGCTACAACATTATCAGAGTGCCCATGGCCAGCTGCGACTTTAGC
ATCAGAACCTATACATACGCCGACACCCCAGATGACTTCCAGCTGCACAACTTCAGCCTGCCT
GAGGAAGATACAAAGCTGAAAATCCCCCTGATCCACCGGGCCCTGCAACTGGCTCAGCGGCCA
GTGTCCCTGCTGGCCTCTCCTTGGACCAGCCCTACCTGGCTGAAGACCAATGGCGCCGTGAAC
GGCAAGGGCTCTCTGAAGGGCCAGCCTGGCGACATCTACCATCAGACCTGGGCCAGATACTTC
GTGAAGTTCCTGGATGCCTACGCTGAGCACAAGCTGCAGTTCTGGGCCGTGACCGCCGAGAAC
GAGCCTTCCGCCGGACTGCTCAGCGGCTATCCTTTCCAGTGCCTGGGCTTCACACCTGAGCAC
CAGCGCGACTTCATCGCCAGGGACCTGGGCCCTACACTGGCCAATAGCACACACCACAACGTG
AGACTGCTGATGCTGGATGATCAGAGACTGCTGCTTCCCCACTGGGCTAAGGTCGTGCTGACC
GACCCTGAAGCCGCTAAGTACGTGCACGGCATCGCCGTGCACTGGTACCTGGACTTCCTGGCC
CCTGCCAAGGCCACCCTGGGAGAAACCCACCGGCTGTTCCCTAACACCATGCTGTTTGCCTCA
GAGGCCTGTGTGGGCTCCAAATTCTGGGAGCAAAGCGTGCGGCTGGGCTCTTGGGACAGAGGA
ATGCAGTACAGCCACAGCATCATCACCAACCTGCTGTACCACGTGGTGGGCTGGACCGACTGG
AACCTGGCCCTGAACCCCGAGGGAGGCCCTAACTGGGTCAGAAACTTCGTGGACAGCCCCATC
ATTGTGGATATCACCAAGGACACGTTCTACAAGCAGCCAATGTTCTACCACCTGGGCCACTTC
AGCAAGTTCATCCCTGAAGGCAGTCAGAGAGTGGGCCTGGTGGCCTCTCAGAAGAACGACCTG
GACGCCGTTGCGCTGATGCACCCCGACGGCAGCGCCGTGGTGGTGGTGCTGAATAGATCTTCT
AAGGATGTGCCTCTGACAATCAAGGACCCTGCTGTGGGATTTCTGGAAACAATCAGCCCTGGA
TACAGCATCCATACATACCTGTGGCGGCGGCAGTGA
```

1. A recombinant adeno-associated virus (rAAV) particle comprising (a) an rAAV vector comprising an expression cassette for expressing a disorder-related polypeptide and/or an RNAi molecule, and (b) a modified AAV9 capsid capable of transducing the cells of the central nervous system (CNS).

2. The rAAV particle of claim 1, wherein the disorder-related polypeptide comprises an ARSA polypeptide, an ABCD1 polypeptide, E3 ubiquitin-protein ligase, FMR1 polypeptide, FMRP polypeptide, MeCP2 polypeptide, hamartin, tuberin, acid alpha-glucosidase, galactosylceramidase, or glucocerebrosidase (GCase).

3. The rAAV particle of claim 1, wherein the modified AAV9 capsid comprises a targeting peptide that has SEQ ID NO: 10.

4. The rAAV particle of claim 1, wherein
the targeting peptide is flanked by linker sequences on its N-terminal end and the C-terminal end.

5. The rAAV particle of claim 4, wherein the combined targeting peptide and linker sequences comprise SEQ ID NO: 11.

6. The rAAV particle of claim 1, wherein
modified capsid protein has a sequence that is at least 98.5% identical to SEQ ID NO: 12.

7. The rAAV particle of claim 6, wherein the modified capsid protein comprises a sequence comprising SEQ ID NO: 12.

8. The rAAV particle of claim 1, wherein
the expression cassette comprises a codon optimized gene encoding for the disorder-related polypeptide and/or the RNAi.

9. The rAAV particle of claim 1, wherein
the rAAV vector comprises a 5′ AAV2 ITR of SEQ ID NO: 4 and a 3′ AAV2 ITR of SEQ ID NO: 5.

10. The rAAV particle of claim 1, wherein
the expression cassette comprises a CMV enhancer element comprising SEQ ID NO: 6.

11. The rAAV particle of claim 1, wherein the expression cassette comprises a chicken β-actin promoter comprising SEQ ID NO: 7.

12. The rAAV particle of claim 1,
wherein the rAAV vector further comprises a WPRE element.

13. The rAAV particle of claim 12, wherein the WPRE element comprises a sequence of SEQ ID NO:8.

14. A method of treating the neurological disorder in a human patient in need thereof, comprising administering to the cerebrospinal fluid (CSF) of the patient a composition comprising an effective amount of recombinant adeno-associated virus (rAAV) viral particle of claim 1.

15. The method of claim 14, wherein the composition is administered directly to the CSF of the patient via intracerebroventricular (ICV) administration.

16. The method of claim 14, wherein the composition is administered directly to the CSF of the patient via direct cisterna magna (dCM) administration.

17. The method of claim 14, wherein the composition is administered directly to the CSF of the patient with an intrathecal microcatheter (IT-CM).

18. The method of claim 14, wherein
the composition is administered only once over the lifetime of the patient.

19. The method of claim 14, wherein
the composition is administered only once yearly to the patient.

20-27. (canceled)

28. A recombinant adeno-associated virus (rAAV) particle comprising (1) a rAAV vector comprising an expression cassette for expressing a disorder-related polypeptide and/or an RNAi, wherein the expression cassette comprises a gene encoding the disorder-related polypeptide and/or the RNAi, wherein the gene is operably linked to a promoter and optionally an enhancer, and (2) a modified AAV9 capsid protein comprising a targeting peptide that has SEQ ID NO: 10 for use in treating a neurological disorder.

29-30. (canceled)

31. The rAAV particle of claim 28,
wherein the modified AAV9 capsid protein is a modified VP3 capsid protein, wherein the modified VP3 capsid protein has a sequence that is at least 98.5% identical to SEQ ID NO: 12.

32-40. (canceled)


SEQUENCE LISTING (23 sequences)

SEQ ID NO: 1 - protein, 507 aa, Homo sapiens
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 2 - DNA, 1530 bp, synthetic construct
  source 1..1530: mol_type = other DNA
atgagcatgggagcccctagatctctgctgctggctcttgctgctggactggctgtggccagacctcctaacatcgtgctgatcttcgccgacgatctcggctatggcgatctgggctgttacggacaccctagcagcaccacacctaacctggatcaactggctgccggcggactgagattcaccgatttctacgtgcccgtgtctctgtgcacacctagtagagctgctctgctgacaggcagactgccagtgcggatgggaatgtatcctggcgtgctggttcctagcagtagaggcggactgcctctggaagaagtgacagttgctgaagtgctggccgccagaggctatctgactggaatggccggaaaatggcacctcggagttggacctgaaggcgcttttctgcctcctcaccagggcttccacagatttctgggcatcccttacagccacgatcagggcccttgccagaacctgacctgctttcctcctgccacaccttgtgatggcggctgtgatcagggactcgtgcctattcctctgctggccaatctgagcgtggaagctcaacctccttggctgcctggcctggaagccagatatatggccttcgctcacgacctgatggccgacgctcagagacaggacagaccattcttcctgtactacgccagccaccacacacactaccctcagttctctggccagtccttcgccgagagatctggcagaggcccttttggcgatagcctgatggaactggatgccgccgtgggaacactgatgacagccattggagatctgggcctgctggaagagacactggtcatcttcaccgccgacaacggccccgagacaatgagaatgagcagaggcggctgtagcggcctgctgagatgtggcaagggaacaacatacgaaggcggcgtcagagagcctgctctggctttttggcctggacatattgcccctggcgtgacacacgaactggcctcttctctggatctgctgcctacactggctgctttggctggcgctcctctgcctaatgtgaccctggatggcttcgatctgtctccactgctgctcggaacaggcaagagccctagacagagcctgttcttctaccctagctaccccgatgaagtgcggggagtgtttgccgtgcggacaggcaagtacaaggcccacttttttacccaaggcagcgcccacagcgataccacagctgatcctgcttgtcacgcctctagcagcctgacagctcatgaaccacctctgctgtacgacctgtctaaggaccccggcgagaactataatctgcttggcggagttgccggcgctacacctgaagttctgcaggctctgaaacagctccagctgctgaaagcccagctggacgctgctgtgacatttggaccttctcaggtggcaagaggcgaggaccctgctctgcagatttgttgtcaccctggctgtacccctagacctgcctgctgtcactgtcctgatcctcacgcttga

SEQ ID NO: 3 - DNA, 10788 bp, synthetic construct
  source 1..10788: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctacgtaccaccatgagcatgggagcccctagatctctgctgctggctcttgctgctggactggctgtggccagacctcctaacatcgtgctgatcttcgccgacgatctcggctatggcgatctgggctgttacggacaccctagcagcaccacacctaacctggatcaactggctgccggcggactgagattcaccgatttctacgtgcccgtgtctctgtgcacacctagtagagctgctctgctgacaggcagactgccagtgcggatgggaatgtatcctggcgtgctggttcctagcagtagaggcggactgcctctggaagaagtgacagttgctgaagtgctggccgccagaggctatctgactggaatggccggaaaatggcacctcggagttggacctgaaggcgcttttctgcctcctcaccagggcttccacagatttctgggcatcccttacagccacgatcagggcccttgccagaacctgacctgctttcctcctgccacaccttgtgatggcggctgtgatcagggactcgtgcctattcctctgctggccaatctgagcgtggaagctcaacctccttggctgcctggcctggaagccagatatatggccttcgctcacgacctgatggccgacgctcagagacaggacagaccattcttcctgtactacgccagccaccacacacactaccctcagttctctggccagtccttcgccgagagatctggcagaggcccttttggcgatagcctgatggaactggatgccgccgtgggaacactgatgacagccattggagatctgggcctgctggaagagacactggtcatcttcaccgccgacaacggccccgagacaatgagaatgagcagaggcggctgtagcggcctgctgagatgtggcaagggaacaacatacgaaggcggcgtcagagagcctgctctggctttttggcctggacatattgcccctggcgtgacacacgaactggcctcttctctggatctgctgcctacactggctgctttggctggcgctcctctgcctaatgtgaccctggatggcttcgatctgtctccactgctgctcggaacaggcaagagccctagacagagcctgttcttctaccctagctaccccgatgaagtgcggggagtgtttgccgtgcggacaggcaagtacaaggcccacttttttacccaaggcagcgcccacagcgataccacagctgatcctgcttgtcacgcctctagcagcctgacagctcatgaaccacctctgctgtacgacctgtctaaggaccccggcgagaactataatctgcttggcggagttgccggcgctacacctgaagttctgcaggctctgaaacagctccagctgctgaaagcccagctggacgctgctgtgacatttggaccttctcaggtggcaagaggcgaggaccctgctctgcagatttgttgtcaccctggctgtacccctagacctgcctgctgtcactgtcctgatcctcacgcttgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 4 - DNA, 145 bp, Adeno-associated virus type 2
  source 1..145: mol_type = unassigned DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 145 bp, Adeno-associated virus type 2
  source 1..145: mol_type = unassigned DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - DNA, 381 bp, synthetic construct
  source 1..381: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 7 - DNA, 323 bp, synthetic construct
  source 1..323: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgcc

SEQ ID NO: 8 - DNA, 594 bp, synthetic construct
  source 1..594: mol_type = other DNA
ttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 9 - protein, 736 aa, Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 7 aa, synthetic construct
KGGGFHG

SEQ ID NO: 11 - protein, 12 aa, synthetic construct
AAAKGGGFHGAS

SEQ ID NO: 12 - protein, 748 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 611 aa, Adeno-associated virus serotype 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 534 aa, Adeno-associated virus serotype 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg

SEQ ID NO: 16 - skipped

SEQ ID NO: 17 - protein, 536 aa, synthetic construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 18 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 19 - DNA, 1611 bp, synthetic construct
  source 1..1611: mol_type = other DNA
atggagttcagctctccgagccgggaagagtgccccaaacccctgtcgagagtgtccatcatggccggcagcctgaccggcctgctgctgctgcaagctgttagctgggccagcggggccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 20 - DNA, 1548 bp, synthetic construct
  source 1..1548: mol_type = other DNA
atggcctctctgtggctgctgagctgcttcagcctggtgggcgccgctttcggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 21 - DNA, 1551 bp, synthetic construct
  source 1..1551: mol_type = other DNA
atgttccagaccggcggactgatcgtgttctacggcctgctggcccagacaatggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 22 - DNA, 1545 bp, synthetic construct
  source 1..1545: mol_type = other DNA
atgctgctgctgctgctgctgctcggcctgagactgcagctgagcctgggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 23 - DNA, 1548 bp, synthetic construct
  source 1..1548: mol_type = other DNA
atgaccatcctgttcctgacaatggtgatcagctacttcggctgcatgaaggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga